SEQUENCE LISTING (46 sequences)

SEQ ID NO: 1 - protein, 2136 aa, Homo sapiens
  misc_feature: Incyte ID No 4936875CD1
MADVTARSLQYEYKANSNLVLQADRSLIDRTRRDEPTGEVLSLVGKLEGTRMGDKAQRTKPQMQEERRAKRRKRDEDRHDINKMKGYTLLSEGIDEMVGIIYKPKTKETRETYEVLLSFIQAALGDQPRDILCGAADEVLAVLKNEKLRDKERRKEIDLLLGQTDDTRYHVLVNLGKKITDYGGDKEIQNMDDNIDETYGVNVQFESDEEEGDEDVYGEVREEASDDDMEGDEAVVRCTLSANLVASGELMSSKKKDLHPRDIDAFWLQRQLSRFYDDAIVSQKKADEVLEILKTASDDRECENQLVLLLGFNTFDFIKVLRQHRMMILYCTLLASAQSEAEKERIMGKMEADPELSKFLYQLHETEKEDLIREERSRRERVRQSRMDTDLETMDLDQGGEALAPRQVLDLEDLVFTQGSHFMANKRCQLPDGSFRRQRKGYEEVHVPALKPKPFGSEEQLLPVEKLPKYAQAGFEGFKTLNRIQSKLYRAALETDENLLLCAPTGAGKTNVALMCMLREIGKHINMDGTINVDDFKIIYIAPMRSLVQEMVGSFGKRLATYGITVAELTGDHQLCKEEISATQIIVCTPEKWDIITRKGGERTYTQLVRLIILDEIHLLHDDRGPVLEALVARAIRNIEMTQEDVRLIGLSATLPNYEDVATFLRVDPAKGLFYFDNSFRPVPLEQTYVGITEKKAIKRFQIMNEIVYEKIMEHAGKNQVLVFVHSRKETGKTARAIRDMCLEKDTLGLFLREGSASTEVLRTEAEQCKNLELKDLLPYGFAIHHAGMTRVDRTLVEDLFADKHIQVLVSTATLAWGVNLPAHTVIIKGTQVYSPEKGRWTELGALDILQMLGRAGRPQYDTKGEGILITSHGELQYYLSLLNQQLPIESQMVSKLPDMLNAEIVLGNVQNAKDAVNWLGYAYLYIRMLRSPTLYGISHDDLKGDPLLDQRRLDLVHTAALMLDKNNLVKYDKKTGNFQVTELGRIASHYYITNDTVQTYNQLLKPTLSEIELFRVFSLSSEFKNITVREEEKLELQKLLERVPIPVKESIEEPSAKINVLLQAFISQLKLEGFALMADMVYVTQSAGRLMRAIFEIVLNRGWAQLTDKTLNLCKMIDKRMWQSMCPLRQFRKLPEEVVKKIEKKNFPFERLYDLNHNEIGELIRMPKMGKTIHKYVHLFPKLELSVHLQPITRSTLKVELTITPDFQWDEKVHGSSEAFWILVEDVDSEVILHHEYFLLKAKYAQDEHLITFFVPVFEPLPPQYFIRVVSDRWLSCETQLPVSFRHLILPEKYPPPTELLDLQPLPVSALRNSAFESLYQDKFPFFNPIQTQVFNTVYNSDDNVFVGAPTGSGKTICAEFAILRMLLQSSEGRCVYITPMEALAEQVYMDWYEKFQDRLNKKVVLLTGETSTDLKLLGKGNIIISTPEKWDILSRRWKQRKNVQNINLFVVDEVHLIGGENGPVLEVICSRMRYISSQIERPIRIVALSSSLSNAKDVAHWLGCSATSTFNFHPNVRPVPLELHIQGFNISHTQTRLLSMAKPVYHAITKHSPKKPVIVFVPSRKQTRLTAIDILTTCAADIQRQRFLHCTEKDLIPYLEKLSDSTLKETLLNGVGYLHEGLSPMERRLVEQLFSSGAIQVVVASRSLCWGMNVAAHLVIIMDTQYYNGKIHAYVDYPIYDVLQMVGHANRPLQDDEGRCVIMCQGSKKDFFKKFLYEPLPVESHLDHCMHDHFNAEIVTKTIENKQDAVDYLTWTFLYRRMTQNPNYYNLQGISHRHLSDHLSELVEQTLSDLEQSKCISIEDEMDVAPLNLGMIAAYYYINYTTIELFSMSLNAKTKVRGLIEIISNAAEYENIPIRHHEDNLLRQLAQKVPHKLNNPKFNDPHVKTNLLLQAHLSRMQLSAELQSDTEEILSKAIRLIQACVDVLSSNGWLSPALAAMELAQMVTQAMWSKDSYLKQLPHFTSEHIKRCTDKGVESVFDIMEMEDEERNALLQLTDSQIADVARFCNRYPNIELSYEVVDKDSIRSGGPVVVLVQLEREEEVTGPVIAPLFPQKREEGWWVVIGDAKSNSLISIKRLTLQQKAKVKLDFVAPATGAHNYTLYFMSDAYMGCDQEYKFSVDVKEAETDSDSD

SEQ ID NO: 2 - protein, 1386 aa, Homo sapiens [sequence omitted]

SEQ ID NO: 3 - protein, 604 aa, Homo sapiens
  misc_feature: Incyte ID No 2181434CD1
MDKLPAIFFLFKNDDVGKRAGSVCTFLEKTETKSHPHTECHSYVFAIDEVLEKVRKTQKRISTKKNPKKAEKLERKKVYRAEYINFLENLKILEISEDCTYADVKALHTEITRNKDSTLDRVLPRVRFTRHGKELKALAQRGIGYHHSSMYFKEKEFVEILFVKGLIRVVTATETLALGIHMPCKSVVFAQDSVYLDALNYRQMSGRAGRRGQDLLGNVYFFDIPLPKIKRLLASSVPELRGQFPLSITLVLRLMLLASKGDDPEDAKAKVLSVLKHSLLSFKRRRAMETLKLYFLFSLQLLIKEDYLNKKGNPKKFAGLASYLHGHEPSNLVFVNFLKRGLFHNLCKPAWKGSQQFSQDVMEKLVLVLANLFGRKYIPAKFQNANLSFSQSKVILAELPEDFKAALYEYNLAVMKDFASFLLIASKSVNMKKEHQLPLSRIKFTGKECEDSQLVSHLMSCKKGRVAISPFVCLSGNTDNDLLRPETINQVILRTVGVSGTQAPLLWPWKLDNRGRRMPLNAYVLNFYKHNCLTRLDQKNGMRVGQLLKCLKDFAFNIQAISDSLSELCENKRDNVVLAFKQLSQTFYEKLQEMQIQMSQNHLE

SEQ ID NO: 4 - protein, 707 aa, Homo sapiens [sequence omitted]

SEQ ID NO: 5 - protein, 358 aa, Homo sapiens
  misc_feature: Incyte ID No 5633694CD1
MRHSLTKLLAASGSNSPTRSESPEPAATCSLPSDLTRAAAGEEETAAAGSPGRKQQFGDEGELEAGRGSRGGVAVRAPSPEEMEEEAIASLPGEETEDMDFLSGLELADLLDPRQPDWHLDPGLSSPGPLSSSGGGSDSGGLWRGDDDDEAAAAEMQRFSDLLQRLLNGIGGCSSSSDSGSAEKRRRKSPGGGGGGGSGNDNNQAATKSPRKAAAAAARLNRLKKKEYVMGLESRVRGLAAENQELRAENRELGKRVQALQEESRYLRAVLANETGLARLLSRLSGVGLRLTTSLFRDSPAGDHDYALPVGKQKQDLLEEDDSAGGVCLHVDKDKVSVEFCSACARKASSSLKIFFFR

SEQ ID NO: 6 - protein, 132 aa, Homo sapiens
  misc_feature: Incyte ID No 7985981CD1
MDLPYYHGRLTKQDCETLLLKEGVDGNFLLRDSESIPGVLCLCVSFKNIVYTYRIFREKHGYYRIQTAEGSPKQVFPSLKELISKFEKPNQGMVVHLLKPIKRTSPSLRWRGLKLELETFVNSNSDYVDVLP

SEQ ID NO: 7 - protein, 802 aa, Homo sapiens
  misc_feature: Incyte ID No 4706628CD1
MWIQVRTIDGSKTCTIEDVSRKATIEELRERVWALFDVRPECQRLFYRGKQLENGYTLFDYDVGLNDIIQLLVRPDPDHLPGTSTQIEAKPCSNSPPKVKKAPRVGPSNQPSTSARARLIDPGFGIYKVNELVDARDVGLGAWFEAHIHSVTRASDGQSRGKTPLKNGSSCKRTNGNIKHKSKENTNKLDSVPSTSNSDCVAADEDVIYHIQYDEYPESGTLEMNVKDLRPRARTILKWNELNVGDVVMVNYNVESPGQRGFWFDAEITTLKTISRTKKELRVKIFLGGSEGTLNDCKIISVDEIFKIERPGAHPLSFADGKFLRRNDPECDLCGGDPEKKCHSCSCRVCGGKHEPNMQLLCDECNVAYHIYCLNPPLDKVPEEEYWYCPSCKTDSSEVVKAGERLKMSKKKAKMPSASTESRRDWGRGMACVGRTRECTIVPSNHYGPIPGIPVGSTWRFRVQVSEAGVHRPHVGGIHGRSNDGAYSLVLAGGFADEVDRGDEFTYTGSGGKNLAGNKRIGAPSADQTLTNMNRALALNCDAPLDDKIGAESRNWRAGKPVRVIRSFKGRKISKYAPEEGNRYDGIYKVVKYWPEISSSHGFLVWRYLLRRDDVEPAPWTSEGIERSRRLCLRLQYPAGYPSDKEGKKPKGQSKKQPSGTTKRPISDDDCPSASKVYKASDSAEAIEAFQLTPQQQHLIREDCQNQKLWDEVLSHLVEGPNFLKKLEQSFMCVCCQELVYQPVTTECFHNVCKDCLQRSFKAQVFSCPACRHDLGQNYIMIPNEILQTLLDLFFPGYSKGR

SEQ ID NO: 8 - protein, 665 aa, Homo sapiens
  misc_feature: Incyte ID No 5790110CD1
MEVSGPEDDPFLSQLHQVQCPVCQQMMPAAHINSHLDRCLLLHPAGHAEPAAGSHRAGERAKGPSPPGAKRRRLSESSALKQPATPTAAESSEGEGEEGDDGGETESRESYDAPPTPSGARLIPDFPVARSSSPGRKGSGKRPAAAAAAGSASPRSWDEAEAQEEEEAVGDGDGDGDADADGEDDPGHWDADAAEAATAFGASGGGRPHPRALAAEEIRQMLQGKPLADTMRPDTLQDYFGQSKAVGQDTLLRSLLETNEIPSLILWGPPGCGKTTLAHIIASNSKKHSIRFVTLSATNAKTNDVRDVIKQAQNEKSFFKRKTILFIDEIHRFNKSQQDTFLPHVECGTITLIGATTENPSFQVNAALLSRCRVIVLEKLPVEAMVTILMRAINSLGIHVLDSSRPTDPLSHSSNSSSEPAMFIEDKAVDTLAYLSDGDARAGLNGLQLAVLARLSSRKMFCKKSGQSYSPSRVLITENDVKEGLQRSHILYDRAGEEHYNCISALHKSMRGSDQNASLYWLARMLEGGEDPLYVARRLVRFASEDIGLADPSALTQAVAAYQGCHFIGMPECEVLLAQCVVYFARAPKSIEVYSAYNNVKACLRNHQGPLPPVPLHLRNAPTRLMKDLGYGKGYKYNPMYSEPVDQEYLPEELRGVDFFKQRRC

SEQ ID NO: 9 - protein, 677 aa, Homo sapiens
  misc_feature: Incyte ID No 2948827CD1
MPALVRKGFDFQRKQYGKLKKFTTVNPEFYNEPKTKLYLKLSRKERSSAYSKNDLWVVSKTLDFELDTFIACSAFFGPSSINEIEILPLKGYFPSNWPTNMVVHALLVCNASTELTTLKNIQDYFNPATLPLTQYLLTTSSPTIVSNKRVSKRKFIPPAFTNVSTKFELLSLGATLKLASELIQVHKLNKDQATALIQIAQMMASHESIEEVKELQTHTFPITIIHGVFGAGKSYLLAVVILFFVQLFEKSEAPTIGNARPWKLLISSSTNVAVDRVLLGLLSLGFENFIRVGSVRKIAKPILPYSLHAGSENESEQLKELHALMKEDLTPTERVYVRKSIEQHKLGTNRTLLKQVRVVGVTCAACPFPCMNDLKFPVVVLDECSQITEPASLLPIARFECEKLILVGDPKQLPPTIQGSDAAHENGLEQTLFDRLCLMGHKPILLRTQYRCHPAISAIANDLFYKGALMNGVTEIERSPLLEWLPTLCFYNVKGLEQIERDNSFHNVAEATFTLKLIQSLIASGIAGSMIGVITLYKSQMYKLCHLLSAVDFHHPDIKTVQVSTVDAFQGAEKEIIILSCVRTRQVGFIDSEKRMNVALTRGKRHLLIVGNLACLRKNQLWGRVIQHCEGREDGLQHANQYEPQLNHLLKDYFEKQVEEKQKKKSEKEKSKDKSHS

SEQ ID NO: 10 - protein, 107 aa, Homo sapiens
  misc_feature: Incyte ID No 1398040CD1
MFFLFFIFLRWSLTLSPRLEGSGMISAHCSLRLLGSSDPPASTSRVAGITGVQHHAWLIFVFLVETGFHHVGQAGLQLLTSGDLPASASQSARITGVSHCAWPSLVF

SEQ ID NO: 11 - protein, 96 aa, Homo sapiens
  misc_feature: Incyte ID No 7716061CD1
MWRLTLLPRLQCSSTISAHYNLCLLDSSDSPASASRVAGISGVHHHAQLIFVFLVETGFHLVGQTGVELLASGDPPALASQSAGITGVSHCAWQYF

SEQ ID NO: 12 - protein, 469 aa, Homo sapiens
  misc_feature: Incyte ID No 6113748CD1
MELPNYSRQLLQQLYTLCKEQQFCDCTISIGTIYFRAHKLVLAAASLLFKTLLDNTDTISIDASVVSPEEFALLLEMMYTGKLPVGKHNFSKIISLADSLQMFDVAVSCKNLLTSLVNCSVQGQVVRDVSAPSSETFRKEPEKPQVEILSSEGAGEPHSSPELAATPGGPVKAETEEAAHSVSQEMSVNSPTAQESQRNAETPAETPTTAEACSPSPAVQTFSEAKKTSTEPGCERKHYQLNFLLENEGVFSDALMVTQDVLKKLEMCSEIKGPQKEVILNCCEGRTPKETIENLLHRMTEEKTLTAEGLVKLLQAVKTTFPNLGLLLEKLQKSATLPSTTVQPSPDDYGTELLRRYHENLSEIFTDNQILLKMISHMTSLAPGEREVMEKLVKRDSGSGGFNSLISAVLEKQTLSATAIWQLLLVVQETKTCPLDLLMEEIRREPGADAFFRARDHPRTCHFRNNPEA

SEQ ID NO: 13 - protein, 132 aa, Homo sapiens
  misc_feature: Incyte ID No 7474037CD1
MDVFQEGLAMVVQDPLLCDLPIQVTLEEVNSQIALEYGQAMTVRVCKMDGEVMPVVVVQSATVLDLKKAIQRYVQLKQEREGGIQHISWSYVWRTYHLTSAGEKLTEDRKKLRDYGIRNRDEVSFIKKLRQK

SEQ ID NO: 14 - protein, 332 aa, Homo sapiens
  misc_feature: Incyte ID No 2955646CD1
MAGPCCVWGVVFFSCLSPAGHGGVNQLGGVFVNGRPLPDVVRQRIVELAHQGVRPCDISRQLRVSHGCVSKILGRYYETGSIKPGVIGGSKPKVATPKVVDKIAEYKRQNPTMFAWEIRDRLLAEGICDNDTVPSVSSINRIIRTKVQQPFHPTPDGAGTGVTAPGHTIVPSTASPPVSSASNDPVGSYSINGILGIPRSNGEKRKRDEDVSEGSVPNGDSQSGVDSLRKHLRADTFTQQQLEALDRVFERPSYPDVFQASEHIKSEQGNEYSLPALTPGLDEVKSSLSASTNPELGSNVSGTQTYPVVTGKGASRRVGALRSVEGASAHAI

SEQ ID NO: 15 - protein, 304 aa, Homo sapiens
  misc_feature: Incyte ID No 1573006CD1
MAQESVMFSDVSVDFSQEEWECLNDDQRDLYRDVMLENYSNLVSMAGHSISKPNVISYLEQGKEPWLADRELTRGQWPVLESRCETKKLFLKKEIYEIESTQWEIMEKLTRRDFQCSSFRDDWECNRQFKKELGSQGGHFNQLVFTHEDLPTLSHHPSFTLQQIINSKKKFCASKEYRKTFRHGSQFATHEIIHTIEKPYECKECGKSFRHPSRLTHHQKIHTGKKPFECKECGKTFICGSDLTRHHRIHTGEKPYECKECGKAFSSGSNFTRHQRIHTEKWITIHFPEICFFTFNCTFWIFLQ

SEQ ID NO: 16 - protein, 595 aa, Homo sapiens
  misc_feature: Incyte ID No 1336756CD1
MRETLEALSSLGFSVGQPEMAPQSEPREGSHNAQEQMSSSREERALGVCSGHEAPTPEEGAHTEQAEAPCRGQACSAQKAQPVGTCPGEEWMIRKVKVEDEDQEAEEEVEWPQHLSLLPSPFPAPDLGHLAAAYKLEPGAPGALSGLALSGWGPMPEKPYGCGECERRFRDQLTLRLHQRLHRGEGPCACPDCGRSFTQRAHMLLHQRSHRGERPFPCSECDKRFSKKAHLTRHLRTHTGERPYPCAECGKRFSQKIHLGSHQKTHTGERPFPCTECEKRFRKKTHLIRHQRIHTGERPYQCAQCARSFTHKQHLVRHQRVHQTAGPARPSPDSSASPHSTAPSPTPSFPGPKPFACSDCGLSFGWKKNLATHQCLHRSEGRPFGCDECALGATVDAPAAKPLASAPGGPGCGPGSDPVVPQRAPSGERSFFCPDCGRGFSHGQHLARHPRVHTGERPFACTQCDRRFGSRPNLVAHSRAHSGARPFACAQCGRRFSRKSHLGRHQAVHTGSRPHACAVCARSFSSKTNLVRHQAIHTGSRPFSCPQCGKSFSRKTHLVRHQLIHGEAAHAAPDAALAAPAWSAPPEVAPPPLFF

SEQ ID NO: 17 - protein, 281 aa, Homo sapiens
  misc_feature: Incyte ID No 71259816CD1
MIKIITSQNIHLLYLDLLDYLKTVLAGYPIELDKLQNLVVNYCSELSDMKIMSQDAMMITDEVKRNMRQREASFIEERRARENRLNQQKKLIDKIHTKETSEKYRRGQMDLDFPSNLMSTETLKLRRKETSTAEMEYQSGVTAVVEKVKSAVRCSHVWDITSRFLAQRNTEENLELQMEDCEERRVQLKALVKQLELEEAVLKFRQKPSSISFKSVEKKMTDMLKEEEERLQLAHSNMTKGQELLLTIQMGIDNLYVRLMGITLPATQQAGVLRGEAHVPG

SEQ ID NO: 18 - protein, 518 aa, Homo sapiens
  misc_feature: Incyte ID No 3354130CD1
MAVALGCAIQASLNQGSVFQEYDTDCEVFRQRFRQFQYREAAGPHEAFNKLWELCCQWLKPKMRSKEQILELLVLEQFLTILPTEIETWVREHCPENRERVVSLIEDLQRELEIPEQQVDMHDMLLEELAPVGTAHIPPTMHLESPALQVMGPAQEAPVAEAWIPQAGPPELNYGATGECQNFLDPGYPLPKLDMNFSLENREEPWVKELQDSKEMKQLLDSKIGFEIGIENEEDTSKQKKMETMYPFIVTLEGNALQGPILQKDYVQLENQWETPPEDLQTDLAKLVDQQNPTLGETPENSNLEEPLNPKPHKKKSPGEKPHRCPQCGKCFARKSQLTGHQRIHSGEEPHKCPECGKRFLRSSDLYRHQRLHTGERPYECTVCKKRFTRRSHLIGHQRTHSEEETYKCLECGKSFCHGSSLKRHLKTHTGEKPHRCHNCGKSFSRLTALTLHQRTHTEERPFKCNYCGKSFRQRPSLVIHLRIHTGEKPYKCTHCSKSFRQRAGLIMHQVTHFRGLI

SEQ ID NO: 19 - protein, 1033 aa, Homo sapiens
  misc_feature: Incyte ID No 1797985CD1
MIEKIAAHLADFTPRLQSNTRALYQYCPIPIINYPQLENELFCNIYYLKQLCDTLRFPDWPIKDPVKLLKDTLDAWKKEVEKKPPMMSIDDAYEVLNLPQGQGPHDESKIRKAYFRLAQKYHPDKNPEGRDMFEKVNKAYEFLCTKSAKIVDGPDPENIILILKTQSILFNRHKEDLQPYKYAGYPMLIRTITMETSDDLLFSKESPLLPAATELAFHTVNCSALNAEELRRENGLEVLQEAFSRCVAVLTRSSKPSDMSVQVCGYISKCYSVAAQFEECREKITEMPSIIKDLCRVLYFGKSIPRVAALGVECVSSFAVDFWLQTHLFQAGILWYLLGFLFNYDYTLEESGIQKSEETNQQEVANSLAKLSVHALSRLGGYLAEEQATPENPTIRKSLAGMLTPYVARKLAVASVTEILKMLNSNTESPYLIWNNSTRAELLEFLESQQENMIKKGDCDKTYGSEFVYSDHAKELIVGEIFVRVYNEVPTFQLEVPKAFAASLLDYIGSQAQYLHTFMAITHAAKVESEQHGDRLPRVEMALEALRNVIKYNPGSESECIGHFKLIFSLLRVHGAGQVQQLALEVVNIVTSNQDCVNNIAESMVLSSLLALLHSLPSSRQLVLETLYALTSSTKIIKEAMAKGALIYLLDMFCNSTHPQVRAQTAELFAKMTADKLIGPKVRITLMKFLPSVFMDAMRDNPEAAVHIFEGTHENPELIWNDNSRDKVSTTVREMMLEHFKNQQDNPEANWKLPEDFAVVFGEAEGELAVGGVFLRIFIAQPAWVLRKPREFLIALLEKLTELLEKNNPHGETLETLTMATVCLFSAQPQLADQVPPLGHLPKVIQAMNHRNNAIPKSAIRVIHALSENELCVRAMASLETIGPLMNGMKKRADTVGLACEAINRMFQKEQSELVAQALKADLVPYLLKLLEGIGLENLDSPAATKAQIVKALKAMTRSLQYGEQVNEILCRSSVWSAFKDQKHDLFISESQTAGYLTGPGVAGYLTAGTSTSVMSNLPPPVDHEAGDLGYQT

SEQ ID NO: 20 - protein, 486 aa, Homo sapiens
  misc_feature: Incyte ID No 2870383CD1
MQSRLLLLGAPGGHGGPASRRMRLLLRQVVQRRPGGDRQRPEVRLLHAGSGADTGDTVNIGDVSYKLKIPKNPELVPQNYISDSLAQSVVQHLRWIMQKDLLGQDVFLIGPPGPLRRSIAMQYLELTKREVEYIALSRDTTETDLKQRREIRAGTAFYIDQCAVHAATEGRTLILEGLEKAERNVLPVLNNLLENREMQLEDGRFLMSAERYDKLLRDHTKKELDSWEIVRVSENFRVIALGLPVPRYSGNPLDPPLRSRFQARDIYYLPFKDQLKLLYSIGANVSAEKVSQLLSFATTLCSQESSTLGLPDFPLDSLAAAVQILDSFPMMPIKHAIQWLYPYSILLGHEGKMAVEGVLKRFELQDSGSSLLPKEIVKVEKMMENHVSQASVTIRIADKEVTIKVPAGTRLLSQPCASDRFIQTLSHKQLQAEMMQSHMVKDICLIGGKGCGKTVIAKNFADTLGYNIEPIMLYQVQCSFLAALGL

SEQ ID NO: 21 - protein, 485 aa, Homo sapiens
  misc_feature: Incyte ID No 1285088CD1
MPAMVEKGPEVSGKRRGRNNAAASASAAAASAAASAACASPAATAASGAAASSASAAAASAAAAPNNGQNKSLAAAAPNGNSSSNSWEEGSSGSSSDEEHGGGGMRVGPQYQAVVPDFDPAKLARRSQERDNLGMLVWSPNQNLSEAKLDEYIAIAKEKHGYNMEQALGMLFWHKHNIEKSLADLPNFTPFPDEWTVEDKVLFEQAFSFHGKTFHRIQQMLPDKSIASLVKFYYSWKKTRTKTSVMDRHARKQKREREESEDELEEANGNNPIDIEVDQNKESKKEVPPTETVPQVKKEKHSTQAKNRAKRKPPKGMFLSQEDVEAVSANATAATTVLRQLDMELVSVKRQIQNIKQTNSALKEKLDGGIEPYRLPEVIQKCNARWTTEEQLLAVQAIRKYGRDFQAISDVIGNKSVVQVKNFFVNYRRRFNIDEVLQEWEAEHGKEETNGPSNQKPVKSPDNSIKMPEEEDEAPVLDVRYASAS

SEQ ID NO: 22 - protein, 751 aa, Homo sapiens
  misc_feature: Incyte ID No 1532441CD1
MVSHGSSPSLLEALSSDFLACKICLEQLRAPKTLPCLHTYCQDCLAQLADGGRVRCPECRETVPVPPEGVASFKTNFFVNGLLDLVKARACGDLRAGKPACALCPLVGGTSTGGPATARCLDCADDLCQACADGHRCTRQTHTHRVVDLVGYRAGWYDEEARERQAAQCPQHPGEALRFLCQPCSQLLCRECRLDPHLDHPCLPLAEAVRARRPGLEGLLAGVDNNLVELEAARRVEKEALARLREQAARVGTQVEEAAEGVLRALLAQKQEVLGQLRAHVEAAEEAARERLAELEGREQVARAAAAFARRVLSLGREAEILSLEGAIAQRLRQLQGCPWAPGPAPCLLPQLELHPGLLDKNCHLLRLSFEEQQPQKDGGKDGAGTQGGEESQSRREDEPKTERQGGVQPQAGDGAQTPKEEKAQTTREEGAQTLEEDRAQTPHEDGGPQPHRGGRPNKKKKFKGRLKSISREPSPALGPNLDGSGLLPRPIFYCSFPTRMPGDKRSPRITGLCPFGPREILVADEQNRALKRFSLNGDYKGTVPVPEGCSPCSVAALQSAVAFSASARLYLINPNGEVQWRRALSLSQASHAVAALPSGDRVAVSVAGHVEVYNMEGSLATRFIPGGKASRGLRALVFLTTSPQGHFVGSDWQQNSVVICDGLGQVVGEYKGPGLHGCQPGSVSVDKKGYIFLTLREVNKVVILDPKGSLLGDFLTAYHGLEKPRVTTMVDGRTSSKSGWTHSIIYKLQR

SEQ ID NO: 23 - protein, 1786 aa, Homo sapiens
  misc_feature: Incyte ID No 3056408CD1
MDPMVMKRPQLYGMGSNPHSQPQQSSPYPGGSYGPPGPQRYPIGIQGRTPGAMAGMQYPQQQMPPQYGQQGVSGYCQQGQQPYYSQQPQPPHLPPQAQYLPSQSQQRYQPQQDMSQEGYGTRSQPPLAPGKPNHEDLNLIQQERPSSLPVEVLASEDAAFGLKDLSGSIDDLPTGTEATLSSAVSASGSTSSQGDQSNPAQSPFSPHASPHLSSIPGGPSPSPVGSPVGSNQSRSGPISPASIPGSQMPPQPPGSQSESSSHPALSQSPMPQERGFMAGTQRNPQMAQYGPQQTGPSMSPHPSPGGQMHAGISSFQQSNSSGTYGPQMSQYGPQGNYSRPPAYSGVPSASYSGPGPGMGISANNQMHGQGPSQPCGAVPLGRMPSAGMQNRPFPGNMSSMTPSSPGMSQQGGPGMGPPMPTVNRKAQEAAAAVMQAAANSAQSRQGSFPGMNQSGLMASSSPYSQPMNNSSSLMNTQAPPYSMAPAMVNSSAASVGLADMMSPGESKLPLPLKADGKEEGTPQPESKSKDSYSSQGISQPPTPGNLPVPSPMSPSSASISSFHGDESDSISSPGWPKTPSSPKSSSSTTTGEKITKVYELGNEPERKLWVDRYLTFMEERGSPVSSLPAVGKKPLDLFRLYVCVKEIGGLAQVNKNKKWRELATNLNVGTSSSAASSLKKQYIQYLFAFECKIERGEEPPPEVFSTGDTKKQPKLQPPSPANSGSLQGPQTPQSTGSNSMAEVPGDLKPPTPASTPHGQMTPMQGGRSSTISVHDPFSDVSDSSFPKRNSMTPNAPYQQGMSMPDVMGRMPYEPNKDPFGGMRKVPGSSEPFMTQGQMPNSSMQDMYNQSPSGAMSNLGMGQRQQFPYGASYDRRHEPYGQQYPGQGPPSGQPPYGGHQPGLYPQQPNYKRHMDGMYGPPAKRHEGDMYNMQYSSQQQEMYNQYGGSYSGPDRRPIQGQYPYPYSRERMQGPGQIQTHGIPPQMMGGPLQSSSSEGPQQNMWAARNDMPYPYQNRQGPGGPTQAPPYPGMNRTDDMMVPDQRINHESQWPSHVSQRQPYMSSSASMQPITRPPQPSYQTPPSLPNHISRAPSPASFQRSLENRMSPSKSPFLPSMKMQKVMPTVPTSQVTGPPPQAPPIRREITFPPGSVEASQPVLKQRRKITSKDIVTPEAWRVMMSLKSGLLAESTWALDTINILLYDDSTVATFNLSQLSGFLELLVEYFRKCLIDIFGILMEYEVGDPSQKALDHNAARKDDSQSLADDSGKEEEDAECIDDDEEDEEDEEEDSEKTESDEKSSIALTAPDAAADPKEKPKQASKFDKLPIKIVKKNNLFVVDRSDKLGRVQEFNSGLLHWQLGGGDTTEHIQTHFESKMEIPPRRRPPPPLSSAGRKKEQEGKGDSEEQQEKSIIATIDDVLSARPGALPEDANPGPQTESSKFPFGIQQAKSHRNIKLLEDEPRSRDETPLCTIAHWQDSLAKRCICVSNIVRSLSFVPGNDAEMSKHPGLVLILGKLILLHHEHPERKRAPQTYEKEEDEDKGVACSKDEWWWDCLEVLRDNTLVTLANISGQLDLSAYTESICLPILDGLLHWMVCPSAEAQDPFPTVGPNSVLSPQRLVLETLCKLSIQDNNVDLILATPPFSRQEKFYATLVRYVGDRKNPVCREMSMALLSNLAQGDALAARAIAVQKGSIGNLISFLEDGVTMAQYQQSQHNLMHMQPPPLEPPSVDMMCRAAKALLAMARVDENRSEFLLHEGRLLDISISAVLNSLVASVICDVLFQIGQL

SEQ ID NO: 24 - DNA, 6790 bp, Homo sapiens
  misc_feature: Incyte ID No 4936875CB1
gcgcggcgggagcagagatctgcggccgtttgcagcttgcggtagggaggcgtggtggtctgaagcctccgagcagccgcggccatggcggatgtaaccgcccgtagtctgcaatacgagtacaaggcgaactcgaatcttgtgctccaagctgaccgttctctcattgaccggacccgccgggatgaacccacaggagaggtgctgtcccttgttgggaagctggagggcacccgtatgggagacaaggctcaacggaccaaaccgcagatgcaggaggaaagaagagccaagcgaagaaagcgtgatgaggaccggcatgacatcaacaagatgaagggttatactctgctgtcggagggcattgatgagatggtgggcatcatctacaagcccaaaactaaagagactcgggagacctatgaggtgctactcagcttcatccaggctgctcttggggaccagccacgtgatatcctttgtggggcagctgatgaagttctagctgttctaaagaatgaaaagctgcgggacaaggaaaggcgaaaggagattgacctgctgctgggtcaaacagatgataccagataccatgtgctagtgaacctgggcaaaaagatcacagactatggtggagataaggaaatccaaaatatggatgacaacattgatgagacatacggtgtgaatgtgcagtttgagtctgatgaggaggaaggtgatgaagacgtatacggggaggttcgagaagaggcatctgatgatgacatggaaggggacgaggctgtcgtgcgctgcaccctctcggctaatctcgtagcctcaggtgaactgatgagttccaagaagaaggatttgcaccctcgggatattgatgcattttggctgcagcggcagctcagtcgtttctatgatgatgccatcgtgtcgcagaagaaggcagatgaagtattggagattttgaagacggccagtgatgatcgggaatgtgaaaatcagctggttctgctgcttggtttcaacacctttgatttcattaaagtgttgcggcagcacaggatgatgattttatactgtaccttgctggccagtgcacaaagtgaagctgaaaaggaaaggattatgggaaagatggaagctgacccagagctatccaagttcctctaccagcttcatgaaaccgagaaggaggatctgatccgagaggaaaggtcccggagagagcgagtgcgtcagtctcgaatggacacagatctggaaaccatggatctcgaccagggtggagaggcactggctccacggcaggttctggacttggaggacctggtttttacccaagggagccactttatggccaataaacgctgtcagcttcctgatggatccttccgtcgccagcgtaagggctatgaagaggtgcatgtgcctgctctgaagcccaagccctttggctcagaagaacaactgcttccagtggaaaagctgccaaagtatgcccaggctgggtttgagggcttcaaaacactgaatcggatccagagtaagctctaccgtgctgcccttgagacggatgagaatctgctgctgtgtgctcctactggtgctgggaagaccaacgtggccctgatgtgcatgctccgagagattgggaaacacataaacatggacggcaccatcaatgtggatgacttcaagattatctacattgcccccatgcgctccttggtgcaggagatggtgggcagctttggaaagcgcctggccacttatggcatcactgttgctgaactgactggggaccaccagctgtgcaaagaagagatcagtgccactcagatcatcgtctgcacccccgagaagtgggacatcatcacccgcaagggtggtgagcgcacctacacccagctggtgcggctcatcattctggatgagattcatcttctccacgatgacagaggtcctgtcttagaagctttagtggccagggccatccgaaacattgagatgacccaagaggatgtccgactcattggtctcagtgccaccctacccaactatgaagatgtagccacctttctacgtgttgaccctgccaagggtctcttttactttgacaacagcttccgtccagtgcctctggaacagacatatgtgggtatcacagagaaaaaagctatcaagcgtttccagatcatgaatgaaatcgtctatgaaaaaatcatggaacatgctggaaaaaatcaggtgctggtgtttgtccactcccggaaggagactggaaagacagccagggccatccgggacatgtgcctagaaaaggacactctgggtctgtttctgagggagggctcagcctccacagaagtcctgcgaacagaagctgagcagtgcaagaacctagagctgaaggatcttctgccttatggctttgctattcatcacgcaggcatgaccagggttgaccgaacactcgtggaggatctttttgctgataaacatattcaggttttagtttccacagcaactctagcttggggtgtgaatctccctgcacatacagtcatcatcaaaggcacccaggtgtacagtccagagaaggggcgttggacagaactgggagcactggacattctgcagatgctgggacgtgccggaagaccccagtatgacaccaagggtgaaggcatactcatcacatctcatggggagctacagtactacctgtccctcctcaatcaacaacttcctattgaaagccagatggtttcaaagcttcctgacatgctcaatgcagaaatcgtgctaggaaatgtccagaatgccaaggatgcggtgaactggctgggctatgcctacctctatatccgaatgctgcgatccccaaccctctatggcatctctcatgatgacctcaagggagatcccctgctggaccagcgccgactagatctggttcatacagctgccctgatgctggacaagaacaatctggtcaagtacgacaagaagacgggcaacttccaggtgacagaactgggccgtatagccagccactactacatcaccaatgatacagtgcagacttacaaccagctgctgaagcccaccctgagtgagattgagcttttcagggtcttctcattgtcctctgagttcaagaacatcacagtgagagaggaggagaagctggagctgcagaagttgctggagagggtgcctatccctgtaaaggagagcattgaggaacccagtgctaagatcaacgttcttctgcaagccttcatctcacagctgaaattggagggctttgcactgatggctgacatggtgtatgtcacacagtcggctggccggttgatgcgagcgatatttgaaattgtcctgaaccgaggttgggcacagcttacagacaagaccctgaacctctgcaagatgatcgacaaacgcatgtggcagtccatgtgtcctctgcgccagttccggaaactccctgaggaagtagtgaagaagattgagaagaagaatttcccctttgagcgtctgtacgacctgaatcataatgagattggggagcttatccgcatgccaaagatggggaagaccatccacaaatatgtccatctgtttcccaagctggagttgtcagtgcacctgcagcctatcacacgctccaccctgaaggtggagctgaccatcacgccagacttccagtgggatgaaaaggtgcatggttcatccgaggctttttggattctggtggaggatgtggacagcgaggtgattctgcaccatgagtattttctcctcaaggccaagtacgcccaggacgagcacctcattacattcttcgtgcctgtctttgaaccgctgccccctcagtacttcatccgagtggtgtctgaccgctggctctcttgtgagacccagctgcctgtctccttccggcacctgatcttgccggagaagtacccccctccaaccgaacttttggacctgcagcccttgcccgtgtctgctctgagaaacagtgcctttgagagtctttaccaagataaatttcctttcttcaatcccatccagacccaggtgtttaacactgtatacaacagtgacgacaacgtgtttgtgggggcccccacgggcagcgggaagactatttgtgcagagtttgccatcctgcgaatgctgctgcagagctcggaggggcgctgtgtgtacatcacccccatggaggccctggcagagcaggtatacatggactggtacgagaagttccaggacaggctcaacaagaaggtggtactcctgacaggcgagaccagcacagacctgaagctgctgggcaaagggaacattatcatcagcacccctgagaagtgggacatactttcccggcgatggaagcagcgcaagaacgtgcagaacatcaacctcttcgtggtggatgaggtccaccttatcgggggcgagaatgggcctgtcttagaagtgatctgctcccgaatgcgctacatctcctcccagattgagcggcccattcgcattgtggcactcagctcttcgctctccaatgccaaggatgtggcccactggctgggctgcagtgccacctccaccttcaacttccatcccaatgtgcgtcccgtccccttggagctgcacatccagggcttcaacatcagccatacacaaacccgcctgctctccatggccaagcctgtgtaccatgctatcaccaagcactcgcccaagaagcctgtcattgtctttgtgccgtctcgcaagcagacccgcctcactgccattgacatcctcaccacctgtgcagcagacatccaacggcagaggttcttgcactgcaccgagaaggatctgattccgtacctggagaagctaagtgacagcacgctcaaggaaacgctgctaaatggggtgggctacctgcatgaggggctcagccccatggagcgacgcctggtggagcagctcttcagctcaggggctatccaggtggtggtggcttctcggagtctctgctggggcatgaacgtggctgcccacctggtaatcatcatggatacccagtactacaatggcaagatccacgcctatgtggattaccccatctatgacgtgcttcagatggtgggccacgccaaccgccctttgcaggacgatgaggggcgctgtgtcatcatgtgtcagggctccaagaaggatttcttcaagaagttcttatatgagccattgccagtagaatctcacctggaccactgtatgcatgaccacttcaatgctgagatcgtcaccaagaccattgagaacaagcaggatgctgtggactacctcacctggacctttctgtaccgccgcatgacacagaaccccaattactacaacctgcagggcatctcccatcgtcacttgtcggaccacttgtcagagctggtggagcagaccctgagtgacctggagcagtccaagtgcatcagcatcgaggacgagatggacgtggcgcctctgaacctaggcatgatcgccgcctactattacatcaactacaccaccattgagctcttcagcatgtccctcaatgccaagaccaaggtgcgagggcttatcgagatcatctccaatgcagcagagtatgagaacattcccatccggcaccatgaagacaatctcctgaggcagttggctcagaaggtcccccacaagctgaataaccctaagttcaatgatccgcacgtcaagaccaacctgctcctgcaggctcacttgtctcgcatgcagctgagtgctgagttgcagtcagatacggaggaaatccttagtaaggcaatccggctcatccaggcctgcgtggatgtcctttccagcaatgggtggctcagccctgctctggcagctatggaactggcccagatggtcacccaagccatgtggtccaaggactcatacctgaagcagctgccacacttcacctctgagcatatcaaacgttgcacagacaagggagtggagagtgttttcgacatcatggagatggaggatgaagaacggaacgcgttgcttcagctgactgacagccagattgcagatgtggctcgcttttgtaaccgctaccctaatatcgaactatcttatgaggtggtagataaggacagcatccgcagtggcgggccagttgtggtgctggtgcagctggagcgagaggaggaagtcacaggccctgtcattgcgcctctcttcccgcagaaacgtgaagagggctggtgggtggtgattggagatgccaagtccaatagcctcatctccatcaagaggctgaccttgcagcagaaggccaaggtgaagttggactttgtggccccagccactggtgcccacaactacactctgtacttcatgagtgacgcttacatgggatgtgaccaggagtacaaattcagcgtggatgtgaaagaagctgagacagacagtgattcagattgagtcctgaggcatttacttttgggtaaaggagagttgagcctgaattaggaatgtgtacattgtaggaatcctggttgtggggaccaggtctgtgggcctcaggtctggccagccagggctggtgctgtccccgcctacctccacttcctttcccttgctcactctggatccagtgacagcaggtgtcatgggtcaagcataaatcatatatagcattttcaggcatgttcctggtagttcttttgagtctgacattctaataaaataatttgtagaaaaaaaaaaaaaaaaaaaa

SEQ ID NO: 25 - DNA, 4859 bp, Homo sapiens
  misc_feature: Incyte ID No 264408CB1
gccaccgcaggctgctccaagtgagaatcgtgagggtggccaagtccagtttggacctctgacccttgggcagcacctcccgacagccggctcgggacccaactctgcgagccaggtgaaaatgagttcttcagtaagaagaaaaggcaagccaggcaaaggaggtggaaaagggtcttctagaggaggaagaggaggcaggagtcacgccagtaaatctcatgggagtggtggcggtggcggtggtggtggtggtggaggtggcggcaacagaaaggcctccagtagaatatgggatgatggagatgacttttgtatcttcagtgaatcaaggcgcccttccagacctagcaacagtaacataagcaaaggagagtcacgcccaaaatggaaacccaaagccaaagtaccccttcagactctacatatgacttctgagaatcaagagaaagtgaaagctcttctccgagacctgcaagaacaagatgctgatgctggatctgaaagaggcctttctggggaggaggaagatgatgagcctgattgctgtaacgatgagcggtactggccagctggacaggaaccttccctcgttcccgacttggatcctttggaatatgctggcttagcctcagtggagccttatgttccagaatttacagtctccccatttgcagtgcaaaaactttccaggtatggtttcaatactgaacgctgtcaagcggtcctgaggatgtgtgatggagatgtgggagcatcactagagcatctccttacccagtgtttttcagagacatttggagagaggatgaagatctctgaggcagtcaaccagataagcttggatgagtgtatggaacagcgacaggaagaggcatttgctctcaagtccatctgtggagaaaaatttatagaaagaattcagaacagagtctggaccattgggttagaactggagtatctgacaagtagattccgcaaatccaagccaaaagaaagtaccaaaaatgtacaagagaattcacttgaaatctgtaaattttacctcaaaggaaattgtaaatttggatcaaaatgcagattcaaacatgaagtgcccccaaatcaaattgttggaagaatagaaagaagtgtagatgattctcatcttaatgctattgaagatgcatcttttttatatgaacttgaaattcgattttctaaagaccacaaatatccctaccaagctccgctcgtggcattttattccaccaatgagaacctacctctggcttgtcgtttacatatttctgagtttctttatgacaaggccttgacatttgcggaaacttcggaacctgtcgtatattctttgataacccttttagaggaagagtcggaaatagtcaagttactaacgaatacccaccacaagtatagtgaccctcctgtgaactttctgccagtaccctctaggaccagaataaataatcctgcctgtcataaaacagtgattccaaataattcttttgtttctaatcaaattccagaagttgaaaaagcatcagaatctgaggagtcagatgaggatgacggtcctgcacctgttatagtagagaatgaaagctatgtgaaccttaagaaaaagatttccaaaagatatgactggcaggcaaagtcagtacatgctgaaaatggtaaaatctgcaagcagttccgaatgaaacaggcttccagacagttccagtccattctgcaagagaggcaatcactccctgcttgggaagaaagagaaaccattcttaacttattgcgtaagcaccaggtggttgtcataagtggtatgactggatgtgggaaaaccacacaaattccgcagtttattctggatgattctctgagtggaccacctgagaaggtagccaacatcatctgtacccaaccccgacgaatctctgcaatctctgttgctgaacgcgttgctaaagaaagagcagagagggtgggtctgaccgtgggataccagattcggttagaaagtgtcaagtcctcagccaccagactgttatactgcaccacgggagtgctgctgagaaggctagaaggagatacagctctacaaggagtttcccatatcattgttgatgaagttcatgagaggacagaagaaagtgacttcttgctgctagttttgaaggacattgtatcgcagaggccaggtcttcaagttattttaatgagtgcaactctaaacgctgagcttttttcagactattttaattcctgccccgttattactataccaggtcgtacatttcctgttgatcaattttttttggaagatgcaattgctgtgacaaggtatgtattacaggatgggagcccatatatgcggtccatgaaacagatttcaaaggaaaagcttaaagcaaggcggaacagaactgcatttgaagaagtggaagaagacctaaggctctcccttcacctccaggatcaggattctgtcaaagatgcagtgccagatcaacagttagattttaagcagctcctggcccgctataaaggggttagcaagtcagtcatcaaaacaatgtccatcatggattttgaaaaggtgaatcttgaattaatagaggccttattagagtggattgtggatggaaagcactcctaccctccaggtgctatacttgtatttttaccaggactagcagaaatcaaaatgctttatgaacagctacagtctaattctcttttcaacaacagacgtagtaatcgatgtgttattcacccacttcattcatctttatccagtgaagagcagcaggctgtgtttgtaaaacctcctgcaggagtaactaagattataatttccaccaacattgctgagacatccataaccatcgatgatgttgtctatgttatcgattctgggaaaatgaaagaaaagagatatgatgccagcaaagggatggaaagtctagaggacacctttgtatctcaagctaatgctctacaaaggaaaggccgagcaggccgtgttgcatctggggtctgcttccatttattcactagccatcactacaatcaccagcttttaaaacaacagctaccagaaatacaaagagtgccattggaacagctgtgtctaagaattaaaattttagagatgtttagtgctcataatctccagtctgtgttctctcggctcattgaacctccacacaccgattctcttcgtgcctcaaaaatacgattacgagacttaggagcattaactccagatgaaagattgacccctcttgggtatcatttggcctctctgcccgtggatgtgagaattggcaaactaatgttgtttgggtctatcttccgctgtttggatcctgctctcaccattgctgccagtttggcttttaagtctccgtttgtatctccctgggataaaaaagaagaagctaaccagaaaaagctggaatttgcattcgcaaacagtgattatctggcccttctacaagcgtataagggatggcagctaagtacaaaagaaggcgtgcgtgcaagttataattactgcagacaaaacttcttgtctggaagagttctgcaggaaatggccagcctcaaacgacaattcacggaactgttatcggatatagggtttgcaagggaagggctcagagcaagggaaattgagaaaagggcccaaggaggagatggtgtcttagatgccacaggagaagaggcaaactcaaatgctgagaaccccaagctgatatcagcaatgctgtgtgctgctttgtatccaaatgtagtgcaggtgaaaagcccagaaggaaaatttcagaagaccagtactggagctgtcagaatgcaaccaaaatcagctgagttgaagtttgtcaccaagaacgatggatatgtacacattcacccttcatcagtgaactatcaggtgagacactttgacagcccctacctgttgtaccacgagaagataaaaactagtcgagtattcatccgagactgcagcatggtgtctgtgtacccgctggtcttgtttggaggaggccaagtgaatgtgcagcttcaaagaggagagttcgttgtctccctggatgatggttggatccgttttgtagctgcttcccatcaggtggctgaactggtaaaggagcttcgttgtgaacttgatcagcttctccaggataaaattaaaaacccaagcattgatctgtgtacgtgtcctcgaggatcccggatcatcagcacaattgtgaaacttgtcaccacacaataaaaagcagtcttagagagtgcttgctactcacctgcttctagctcacctgggaaataacagcagaacctctacctcgaactaaagacctattggggctggccctggtggaggagcccagggcatgaagcccaaggcagctgaggcagtgtatatacccttagggccatttctaacaaagccttggccactcccagcacaatttggagtgtcaagggtgagagcctaaaacccagcttgcctgtctttgtctctgtgattgttctggagtgaattaagttcacctgataactcaaaagtgaatgtataatacaattctgttttaatctgtgtattctttttctcctactttttactggggtgagaggggcatgaagagaaatacgccttttttttttttcttttcctgtcgccaaggctcgactgagagaagtcagaacagagaaggggaaaaaaaacccaaaattatgtgaacaagcaaaattaaaatttcattttaggctattggctactgagtaaacttgacttgtgaggggtttttatttttactcattaaaagtcaacttaaaaaaaaaaaaaa

SEQ ID NO: 26 - DNA, 3336 bp, Homo sapiens
  misc_feature: Incyte ID No 2181434CB1
tggaataaaatagatttatgagcgtagtagctcggaatcggctcgagattgttagtcaaaagatatggtgaaaatgtttcctcttctgttgaaaagttaagagaaatggataagttgcctgcaatattttttttgtttaagaatgatgatgtgggaaaaagagctggaagtgtgtgcacttttctggagaagacagagacaaaaagccatccccacactgaatgtcatagttatgtctttgcaatagatgaagtacttgaaaaagtgaggaagacacagaaaaggattagcactaaaaaaaacccaaagaaggctgaaaaactggaaagaaaaaaagtgtatagagctgaatatattaatttcctggagaatctgaagattctggaaatttctgaggactgcacgtatgctgatgtcaaagccctacacactgaaattaccaggaataaagactcaactttggatagggtattaccgcgagtgcgatttacaagacacggcaaagaactgaaggctttagcacaaagggggattggatatcatcacagcagcatgtattttaaagaaaaagagtttgttgagatactctttgtaaaagggcttattagggtagtgacagctactgaaacacttgccttagggatccacatgccatgcaaatctgttgtttttgcccaagactcagtctatctggatgctttaaattacagacagatgtctggtcgtgctggaagaagaggtcaagacctgcttggaaatgtgtatttctttgatatcccattgcccaaaataaaaagactccttgcatccagtgttcctgagctgagaggacagttccctctcagcataaccctggtcctgcgactcatgctgctggcttccaagggagatgacccagaggatgccaaggcaaaggtgttgtcagtgctaaagcattcattgctgtcttttaagagacgaagagccatggagactttgaaactttactttttgttttccttgcagctccttatcaaagaggactatttaaataaaaagggtaatccaaagaaatttgcaggacttgcatcatatttgcatggtcatgaaccttcaaatcttgtttttgtaaattttctcaagagaggccttttccataatctctgtaagccagcctggaaaggctcacaacaattttcccaagatgtgatggaaaagctcgtgttagtattggcaaatttgtttggaagaaaatatattccagcaaaattccaaaatgctaatttaagtttttctcagtcaaaggtgatccttgccgaactcccggaggattttaaagctgctttatatgagtataacctggcagtaatgaaggattttgcctccttcctgctgattgcttccaagtcggtgaacatgaaaaaagagcatcaactccctttgtcaagaatcaaattcacaggtaaagaatgtgaagactcccaactcgtgtctcacttgatgagctgcaagaaaggaagagtagccatttcaccatttgtttgtctttcggggaacacagataatgatttgcttcgaccagagactatcaaccaggtcatcctgcgcacagtcggtgttagtggcactcaggctcctctgctgtggccatggaaattagataaccgaggaaggagaatgccactaaatgcatatgtgctcaatttctataaacacaactgcttgacaagattagaccaaaaaaatgggatgcgtgtgggacagcttttaaagtgtttgaaagattttgcattcaacattcaggctatcagtgactccttgagtgaactatgtgaaaataagcgtgacaatgtagtcctggcatttaaacaattgagtcaaaccttttatgagaaacttcaagaaatgcaaattcaaatgagtcaaaatcatttagaataacaccatggaaaactttcaagtctgattatgtggtatttatccctttgcaaggagagatataattaagcttacacaatgaaatggaaaaaatgtttgtcttggagtcaaacagaattaaactcagataccagctctgctattttctaactgaatgactttaagttatgtaatatatctgagctttaacttcatttttggcaaaaccagagtaaaaatgaatacctctagttgttttgaggattaaatgagataatgtaagaaaagtgattgggattgggtggtgacttaatgaacggtagtggttttttaagtagttaatgtatagcaaaattagtttcacattgtcaagttttcaatacatccccaagttaattgaattttaaattaatgatcaataaatcacaaaggacccaaatcaattctgaacaacaatttagttatgtaagaagacttctgagattacaagaaactcaccgctgtggactggatgtttgtgccctcccctccaaaatttttatattgaaattctaaccctcaatgtgatggtattaggagatgataggtcatgagggtggagctccttggatgtaattagtgcctttaacagagagacaagagagcttgttctccaatctctgctcactaccactggatgatacaatgggaagatggccatctgcagaccaagaagcaagccctcaacagaactgaatctacttacaccatgatcttgaactttccagcctccaggattgtgagaaatacatgttgttgtttagccatctagtctgtggttttctgttgaagcagtctgaattgactaaaacagtcacttggagtagttataaaccactttcctgttgaaagcagaacatgctgattcaactgtttgttcaatagcaatgatagatttgtttaagtcccctacactttcttatttctaaatgatcaagagtacacttcctggcagtgattaaggagtgtgtatctaacagaaaaaatatatataccctgtgaacccgaatatggaattcagattgtttctgccctcagtatcatacttaaaaaacaagcatacaaacaaacataagggaacaaacagcaaccataacaaaaacaaacctttaaaggtgggtttttgctgtgataaatgaatacggtactctgaaggagaaaaaagtttctcaaatgagcttaaactgcaagtgatttaaaaattagagaatataattcttaaagctattgaaagtttcaaccagaaaacctcaagtgaattttgtatgtaaatgaaatcttgaatgtaagttctgtgattctttaagcaaacaattagctgaaaacttggtattgttgtagtttatgtagtaagtgacttggcacccatcagaaaataaagggcattcaaatcgaacacac

SEQ ID NO: 27 - DNA, 2918 bp, Homo sapiens
  misc_feature: Incyte ID No 1367252CB1
gcagaacttgaaggttaaaccactagcccatttcacagaatgtttcatccatttgtggaccaaaagatggagttggtttttatttttaaaaagataatgttaatgatctgataccactacaaatatttacgtgagaagattcatggacttgtcttttggttggactgtcactcatttctgaaagtttcttcagccacaatttctatttgaaaattcaagtatcaaaggataccaggtttagaatggtataatgatgtattttgtctgaggactgcaaattttatagagaccacagttggattccagtgatattctgcaatcaaagtgatttgataaacctaattttgaagcattttatatttataagcgacatcaaaagatgggagaaaaaaatggcgatgcaaaaactttctggatggagctagaagatgatggaaaagtggacttcatttttgaacaagtacaaaatgtgctgcagtcactgaaacaaaagatcaaagatgggtctgccaccaataaagaatacatccaagcaatgattctagtgaatgaagcaactataattaacagttcaacatcaataaaggatcctatgcctgtgactcagaaggaacaggaaaacaaatccaatgcatttccctctacatcatgtgaaaactcctttccagaagactgtacatttctaacaacaggaaataaggaaattctctctcttgaagataaagttgtagactttagagaaaaagactcatcttcgaatttatcttaccaaagtcatgactgctctggtgcttgtctgatgaaaatgccactgaacttgaagggagaaaaccctctgcagctgccaatcaaatgtcacttccaaagacgacatgcaaagacaaactctcattcttcagcactccacgtgagttataaaaccccttgtggaaggagtctacgaaacgtggaggaagtttttcgttacctgcttgagacagagtgtaactttttatttacagataacttttctttcaatacctatgttcagttggctcggaattacccaaagcaaaaagaagttgtttctgatgtggatattagcaatggagtggaatcagtgcccatttctttctgtaatgaaattgacagtagaaagctcccacagtttaagtacagaaagactgtgtggcctcgagcatataatctaaccaacttttccagcatgtttactgattcctgtgactgctctgagggctgcatagacataacaaaatgtgcatgtcttcaactgacagcaaggaatgccaaaacttcccccttgtcaagtgacaaaataaccactggatataaatataaaagactacagagacagattcctactggcatttatgaatgcagccttttgtgcaaatgtaatcgacaattgtgtcaaaaccgagttgtccaacatggtcctcaagtgaggttacaggtgttcaaaactgagcagaagggatggggtgtacgctgtctagatgacattgacagagggacatttgtttgcatttattcaggaagattactaagcagagctaacactgaaaaatcttatggtattgatgaaaacgggagagatgagaatactatgaaaaatatattttcaaaaaagaggaaattagaagttgcatgttcagattgtgaagttgaagttctcccattaggattggaaacacatcctagaactgctaaaactgagaaatgtccaccaaagttcagtaataatcccaaggagcttactatggaaacgaaatatgataatatttcaagaattcagtatcattcagttattagagatcctgaatccaagacagccatttttcaacacaatgggaaaaaaatggaatttgtttcctcggagtctgtcactccagaagataatgatggatttaaaccaccccgagagcatctgaactctaaaaccaagggagcacaaaaggactcaagttcaaaccatgttgatgagtttgaagataatctgctgattgaatcagatgtgatagatataactaaatatagagaagaaactccaccaaggagcagatgtaaccaggcgaccacattggataatcagaatattaaaaaggcaattgaggttcaaattcagaaaccccaagagggacgatctacagcatgtcaaagacagcaggtattttgtgatgaagagttgctaagtgaaaccaagaatacttcatctgattctctaacaaagttcaataaagggaatgtgtttttattggatgccacaaaagaaggaaatgtcggccgcttccttaatcatagttgttgcccaaatctcttggtacagaatgtttttgtagaaacacacaacaggaattttccattggtggcattcttcaccaacaggtatgtgaaagcaagaacagagctaacatgggattatggctatgaagctgggactgtgcctgagaaggaaatcttctgccaatgtggggttaataaatgtagaaaaaaaatattataaatatgtaactaacgcctgtttgtgaaattagcttatcaggctgaaattaaagccatgcaaaagaaggtctaggtccatcaaggaaattcccctccgttttcctttgtcatggggtttatgttttatttcagattttatttgtgtgacttagaaattccaggaacacaattaggatattttcatacacatagggtatcttgttcactgctgtgctactttacatgagtaggatggaagtgtatattttatatgaaataccactgtacaatttataatttatttacaaattatatattaagagaaacaaatgtcataacagaactcagctgtttctaattgcttttgtgactgttaccttttagttcatgcccccccaaagagctaaatttcacatttttacctacaaaattgattt

SEQ ID NO: 28 - DNA, 1610 bp, Homo sapiens
  misc_feature: Incyte ID No 5633694CB1
cgttgtctgggtggcgcggtcgagtcatcgcagggcctcaccgcttcgttctcccgtccctccccgcgccttggcgcggggggtcgactagccaagtgaggcgggaggcgactcggacctttccctgcatttcgtttcggccagtgccgggggctacccgccctggggcctgggatccttggggcccgtgaggcccactcttagcggccggggcctaccgcggcccgccgctggccctcatgaggcatagcctgaccaagctgctggcagcctcgggcagcaactccccaacccgcagtgagagcccggagccggctgcaacttgttcgctgccctctgacctgacccgggctgcagcgggggaggaggagacggcggcggccggatctcccggccgcaagcagcagtttggcgacgaaggagagttggaagccgggagggggagccgcggcggcgtggccgtgcgcgcgccctcccccgaggagatggaggaggaggcgatcgccagcctcccgggggaagagacggaggatatggactttctgtctgggctggaactggcggatctcctggaccccaggcaaccggactggcacctggaccccgggcttagctcgccggggcctctctcctcgtctggcggaggctcggatagcggcggcctgtggagaggggacgatgacgatgaggccgcggctgctgaaatgcagcgcttctctgacctgctgcaaaggctgttaaacggtatcggaggctgcagcagcagcagtgacagtggcagcgccgaaaagaggcggagaaagtccccaggaggaggcggcggtggcggcagcggtaacgacaacaaccaggcggcgacaaagagtccccggaaggcggcggcggccgctgcccgccttaatcgactgaagaagaaggagtacgtgatggggctggagagtcgagtccggggtctggcagccgagaaccaggagctgcgggccgagaatcgggagctgggcaaacgcgtacaggcactgcaggaggagagtcgctacctacgggcagtcttagccaacgagactggactggctcgcttgctgagccggctgagcggcgtgggactgcggctgaccacctcgctcttcagagactcgcccgccggtgaccacgactacgctctgccggtgggaaagcagaagcaggacctgctggaagaggacgactcggcgggaggagtctgtctccatgtggacaaggataaggtgtcggtggagttctgctcggcgtgcgcccggaaggcgtcgtcttctcttaaaattttcttttttaggtgatttccttcctgccaggctccgttgtaggggttacagaacagtcgttcccgcctcacaacctgtggatacagctgttggggcagaagagacgggaccagctgctggccacatttcctgctttattttaaaagcttagcagtgtctgcaaaaacgaatcttttcctacaacctgttaactgactggactgttggtaacaaagtaattgtgagaaccatgtcggtcaaaaatttggcatctgctgaaaaaaatgaatgccattttcaagttcccaaattacttctatactgatttca

SEQ ID NO: 29 - DNA, 935 bp, Homo sapiens
  misc_feature: Incyte ID No 7985981CB1
gagaggaagaggtagctccacaggaggtacagctgcttacacatctctcctcagagctgtcccttgacttgggggtgaatttcaggccaacagggcttcctgggatacaagagcgttctccatggatctgccttactaccatggacgtctgaccaagcaagactgtgagaccttgctgctcaaggaaggggtggatggcaactttcttttaagagacagcgagtcgataccaggagtcctgtgcctctgtgtctcgtttaaaaatattgtctacacataccgaatcttcagagagaaacacgggtattacaggatacagactgcagaaggttctccaaaacaggtctttccaagcctaaaggaactgatctccaaatttgaaaaaccaaatcaggggatggtggttcaccttttaaagccaataaagagaaccagccccagcttgagatggagaggattgaaattagagttggaaacatttgtgaacagtaacagcgattatgtggatgtcttgccttgaagataaggctgccggacaaagcaagttgaagagatgagtaacagttctcactgatgacccacttctgcaggcataggtccagagcaccaaactctagtggacaattcagactctcctggttgtgtaactgaagatgttctgccaaccagcaccagaggtcactctccaaatccccgctcccagacatataccaggagcaatttcaaaagcctctccagtttcactcttctttcttgggaatgggacagctgaacattttcctttgactggttaaggttaccaccttacatcatggtgacaccctcctttggaccatgcaggtcagaggggcagtttatacagaggaggggcacacttgcctggggagttgaagcctgagttccagtccgtggtgagtgaacttggaccggttccg

SEQ ID NO: 30 - DNA, 3609 bp, Homo sapiens
  misc_feature: Incyte ID No 4706628CB1
ccgtgacctccatgtgggagctccagctctataagtaaacactctgcgcggcgcagacatggcctcttcctatctttgaggcggtgtctgcggcagcgcctcagagtggttccggtcgtctctcctcaagtcggctagtcgggcgcgcgcgctgagagtcgtcgccgcctgtcgggcccggcgtccggtcggtccggtgggcgcgctcgcccgcctgccgctgagggcccgagccgcagggaaagcggcgcgggccgggcggggcgcggcgcccagagctcagggggagacaaaggggaccggttcctctctaggcgccaagatgtggatacaggttcgcaccattgatggctccaagacgtgcaccattgaggacgtgtctcgcaaagccacgattgaggagctgcgcgagcgggtgtgggcgctgttcgacgtgcggcccgaatgccagcgcctcttctaccggggcaagcagttggaaaatggatataccttatttgattatgatgttggactgaatgatataattcagctgctagttcgcccagaccctgatcatcttcctggcacatctacacagattgaggctaaaccctgttctaatagtccacctaaagtaaagaaagctccgagggtaggaccttccaatcagccatctacatcagctcgtgcccgtcttattgatcctggctttggaatatataaggtaaatgaattggtggatgccagagatgtcggccttggtgcttggtttgaagcacacatacatagtgttactagagcttctgatggacagtcacgtggcaaaactccactgaagaatggcagttcttgtaaaaggactaatggaaatataaagcataaatccaaagagaacacaaataaattggacagtgtaccctctacgtctaattcagactgtgttgctgctgatgaagacgttatttaccatatccagtatgatgaatacccagaaagcggtactctagaaatgaatgtcaaggatcttagaccacgagctagaaccattttgaaatggaatgaactaaatgttggtgatgtggtaatggttaattataatgtagaaagtcctggacaaagaggattctggtttgatgcagaaattaccacattgaagacaatctcaaggaccaaaaaagaacttcgtgtgaaaattttcctggggggttctgaaggaacattaaatgactgcaagataatatctgtagatgaaatcttcaagattgagagacctggagcccatcccctttcatttgcagatggaaagtttttaaggcgaaatgaccctgaatgtgacctgtgtggtggagacccagaaaagaaatgtcattcttgctcctgtcgtgtatgtggtgggaaacatgaacccaacatgcagcttctgtgtgatgaatgtaatgtggcttatcatatttactgtctgaatccacctttggataaagtcccagaagaggaatactggtattgtccttcttgtaaaactgattccagtgaagttgtaaaggctggtgaaagactcaagatgagtaaaaagaaagcaaagatgccgtcagctagtactgaaagccgaagagactggggcaggggaatggcttgtgttggtcgtacgagagaatgtactattgtcccttctaatcattatggacccattcctggtattcctgttggatcaacttggagatttagagttcaggtgagcgaagcaggtgttcacagaccccatgttggtggaattcatggtcgaagtaatgatggggcttattctcttgtactggctggtggatttgcggatgaagtcgaccgaggtgatgagttcacatacactggaagcggtggtaaaaatcttgctggtaacaaaagaattggtgcaccttcagctgatcaaacattaacaaacatgaacagggcattggccctaaactgtgatgctccattggatgataaaattggagcagagtctcggaattggagagctggtaagccagtcagagtgatacgcagttttaaagggaggaagatcagcaaatatgctcctgaagaaggcaacagatatgatggcatttataaggtggtgaaatactggccagagatttcatcaagccatggattcttggtttggcgctatcttttaagaagagatgatgttgaacctgctccttggacctctgaaggaatagaacggtcaaggagattatgtctacgtttacagtatccagcaggttacccttcagataaagaagggaagaagcctaaaggacagtcaaagaagcagcccagtggaaccacaaaaaggccaatttcagatgatgactgtccaagtgcctccaaagtgtacaaagcatcagattcagcagaagcaattgaggcttttcaactaactcctcaacagcaacatctcatcagagaagattgtcaaaaccagaagctgtgggatgaagtgctttcacatcttgtggaaggaccaaattttctgaaaaaattggaacaatcttttatgtgcgtttgctgtcaggagctagtttaccagcctgtgacaactgagtgcttccacaatgtctgtaaagattgcctacagcgctcctttaaggcacaggttttctcctgccctgcttgccggcatgatcttggccagaattacatcatgattcccaatgagattctgcagactctacttgaccttttcttccctggctacagcaaaggacgatgatctgcctgctttcactgtgttgttcatggtggctttttggacaataaagaatctaaaatgggtggggagggtggaagaaatggtggactgtatctctcacgttctgaagcagctaatcctctttcccacatagccatcatcttgtgtgtgtagtaagaggcccatttctcaactgtcttttaaatatctaaaggtagttcctgtaacaactagttttaatgagtaaaaagtcaaagcctcagctctagttgatatccaagttatgatttattttgcaactacctcaggacagaaaagatttatggggattttaaaaatcattgaataactagttaaatgaaattttagctacacactgcctcccaaatattagttgtgcctggttcttgtaatttgattttacagaaaaggaaatgacacttgagatccttggaatgaacacagcttctaaagtgtgcatatacttttttaacgtctcttcttccattacaatgtgtgttttgcaaggacaggttcattttttttagcccactttgtgaactccattgtgcttttttctggtgttttatgcaagttgactactaatgactaatgagaacaataatgaatgcattgttgctgcattagtgtaatgtggtgtggttttgcacttaaaagaggtattcatatgctctagttgtaaatgttcatgaaaatccacttctctactagtcgaactgcttttagtgtctcaccagtggttttacatctgcagagttttgagggctgtgctgacctttgagaggatttgaaattgcttcatattgtgatcctaaattttatattcactatattccctaaagtataccttaataaatattttatgatcaaaaaaaaaa

SEQ ID NO: 31 - DNA, 4136 bp, Homo sapiens
  misc_feature: Incyte ID No 5790110CB1
tttctccacctcttccctgcactgaaaaaaggccattttctccagctggttgctgttataacacgtgttgaattcttccagcctcttcacttttaacatcttagtagtgagtccgattaagctactttctggagtgtggttttcgtctccctctgtctccgatttataaatcaggacacaattatgactcttcagtgcttctttctcttgaactgcaccgccgagcgcctccgccgccggggcaaacggccacgaactacacttcccgacacgccgcgtgaggcgctgccagcggccggccgagggcgggcggacgcgggagctgcggacgtgaggcatgagcggcgccctcctccggcccgcgagcgtcctgctggttccccgagcgagggtctcgcggcgcggggcctagcggagggcatcgaaggcctccgcgtgcgcacgggttgctgcggccgcgccgggcgccggggagggcggcggccgccatggaggtgagcgggccggaagacgaccccttcctttcgcagctgcaccaggtgcagtgccccgtgtgccagcagatgatgcccgccgcgcacatcaactcgcacctggaccgctgtctgctgctccacccggcggggcacgcggagcccgcggccgggtcgcaccgcgccggggagcgggccaaggggccctcgccgcccggcgccaagaggcggcggctgtcggagagctcggcgctgaagcagccagccaccccgacggcagccgagagcagcgagggcgagggtgaggagggcgacgacggcggcgagaccgagagccgcgagagctacgacgcgccgcccacacccagcggcgcccgccttatccccgacttcccggtggcccgctccagcagccccgggaggaaggggtcggggaagaggccggcggccgccgccgcggcggggagcgcgtctccgcgcagctgggacgaggcggaggcgcaggaggaggaggaggccgtgggcgacggcgatggcgacggggacgcggacgcggacggcgaggacgacccggggcactgggacgcggacgctgccgaagccgccaccgccttcggggccagcggcgggggccgcccgcacccccgggcgctggctgccgaggagatccgacagatgctacagggcaagccgctggccgacacgatgcgtcctgacacgctgcaggattacttcgggcagagcaaggccgtgggccaggataccctgctgcgctcgctcctggagaccaacgaaatcccctcgcttatcctgtgggggccgccgggctgcggcaagaccactctggctcacatcatagccagcaacagcaagaaacatagcataaggtttgtgacattatctgcaacaaatgccaagacaaatgatgtgcgagatgtcataaaacaagctcaaaatgaaaagagctttttcaaaaggaaaaccatcctttttattgatgagattcatcggttcaataaatctcagcaggacactttccttcctcacgtggaatgtgggacgatcactctgattggggcaaccactgaaaacccttccttccaggtcaacgctgctcttctgagccgctgtcgagtgattgttcttgagaagcttccagtagaggcaatggtgactattttaatgcgagcgatcaactccctgggaatccacgtcctagactctagccgtcccactgaccctctgagccacagcagcaacagcagctcagagcccgccatgttcatagaggataaagcagtagacaccctggcttacctcagtgacggtgacgcccgagctgggttgaacggactgcagctggcggtgctggctaggttaagctctaggaagatgttctgtaagaagagtgggcaatcctattctcccagtagagttctgatcacagagaatgacgtgaaggagggcctacagcgatcccacattttatatgaccgggcaggtgaggagcattacaactgcatctccgccctgcacaagtccatgcggggctcagaccagaacgcctccctctactggctggctcgcatgctcgagggaggagaggacccactctacgtggcacggaggcttgtcaggtttgccagcgaggacataggtctggcagacccatctgcgttaacacaagcggttgctgcctaccaaggctgtcattttataggcatgcctgaatgtgaggtgcttctggcccagtgtgtggtctactttgccagagccccaaagtccattgaggtgtacagcgcctacaacaacgtcaaagcctgcctgaggaaccaccaggggccactgccccccgtgcccctgcacctgaggaacgcgcccactaggctgatgaaggatttgggctatggcaaaggctacaagtacaaccccatgtacagcgagcctgtggatcaggagtacctgcctgaagagttgaggggggtagatttcttcaagcagaggaggtgctgactcctcagggcacgacagcagaaggatgttgcttttttaagggagggccagaaagaaagttagtggattgcaaagttggttgcctggtggaagttagaacagaccaacattttgtgccagaaatttaagagttccataggtggaggcgcagttctttcgaataaatgtgtaactttgaaattgtgttcatttgcactcggtgcagcggttatgcttatgaaaatacctggcagctttgtgcaatgaattaatgttataaggaattatctattttgtcatagtatttaagtcataatgtcatttcagaattcagttctgtaggattttcttttctttaaaaaatgtatattctgggtagttttaattggtaaaaaaatgtaattgtgatttaatactgcatagtgttttgggtattttttttatatgcaaaggtcttacgagccaataaaactatttcaaagtactcttcgattctgtcatggttttcctgcctggatgctaggtaccagcgttgtcaccattgcatttggtgggtggatactgggaaggagaaatcaccccagatgggaagagtggggagcttaagttaagaagtcagtgttatcttgttgaaagttaactctgatctctttaaaggaatacataaaggaattctttaaatggcttgtggaagactccagtagtcccatgcccattttttcccacttgtcctggtttcttcttgcagctccatatttctaaacagtcgtttttcttttactttatgtgtgtcctgaacacaaaatacgccactccttctgctcagttaagagttatttgtccctactgctacttcctctccctctccttagttgcatgtcgtgcatatgcccacaaggatggccccttcaggtagtcggttctcctcctgccgttgcgtgatccctcctggggtcctcctgccatgtctggaaggctgccggctgttggcctgggacgtcctctgcctttattctgagtgagatgcccattttctggatgctccatcccgccttactggtttattccgctatttcagtgcaacccatcctctagtagttttatcaggctggagggactgtatgttgcagaggcttggcccgactggcctccctccttctgtctggagtggttgcactgcagtgtgcagttgccatccccagaatctcccttcatcatcaccctggagctttcctctgcttctccctgcaccccatctgatgacaggactgactgctttctggcacaggtggcatgtaccctctagtagctttatggagcaggggtgtgtggaggaaaagtatttttagacctcaggagactcaaaatgcttttattgtaccttcagacgtgtctgagggtttggctagtaataaattgtaggtaagaaattgtgcattgttttacactttcccacattgttgtcaagtttgaaggcatcctgatgtgtgtggtcttttgtttgtaacctctatcaacttgtagactcttctgtctttgctccccgtgttttaaaagtctgcactgggtacttgttgggcctttcaatctggaaactcctttcagttctagggaagtttctttttttattcaataaatttatttatttatgaaaaaacctcgtgccgaatt

SEQ ID NO: 32 - DNA, 2850 bp, Homo sapiens
  misc_feature: Incyte ID No 2948827CB1
ttgaggacgtgactccaggatattcaacacaggaaggagctcgacctggcatggttttaagtgatattaagagtattggcttatatttaagaagtcaaaagataccactttatgaggaatgccagctttggtgagaaaaggatttgattttcagagaaaacagtatggcaaactaaagaagtttactactgtaaatcctgagttttataatgaaccaaaaaccaaactttatcttaagctaagtcggaaggaaagatcttcagcttatagcaaaaatgatctttgggtggtttcaaaaaccctagactttgagctggatacttttatcgcatgtagtgctttctttggaccatcatctatcaatgagatagaaatactgcctttgaaaggctatttcccttctaattggcccactaacatggttgtccatgcgttattggtttgtaatgctagcacagaactgactactttgaaaaacattcaggactactttaatccagctactctacctctaacacagtacctgttaacaacgtcttcgccaactatagttagtaacaaaagagtcagtaagagaaaatttatcccaccagccttcacaaatgtcagtacaaaatttgaactactcagcctaggagcaacattgaagttagctagtgagttgattcaggtacacaagttaaacaaggatcaagctacagctctaattcaaatagctcaaatgatggcatcacatgaaagcattgaagaagtgaaggaactgcaaactcataccttccctatcacaatcatacatggtgtgtttggagcaggaaagagttacttgctggcagtggtgattttgttctttgtacagctgtttgaaaagagtgaagctcccaccattggaaatgcaaggccgtggaaacttctgatttcttcttctactaatgtggctgttgacagagtacttcttgggcttctcagtcttggatttgaaaactttatcagagttgggagtgttaggaagattgccaaaccaattttaccttatagcttgcatgctggctcagaaaatgaaagtgaacagttaaaagaactacatgcactaatgaaagaagacctgactcctacggaaagagtctatgtgagaaaaagcattgagcagcataaactggggaccaatagaaccctgctgaagcaggttcgagtagttggagttacctgtgcagcctgcccattcccatgcatgaatgatcttaaatttcctgtagttgtgctggatgagtgtagtcagataactgaaccggcctctctccttcccattgcaaggtttgagtgtgaaaagctgattcttgttggggatcccaaacagctacctcctactattcagggttctgatgcagctcatgaaaatggattggaacaaactctttttgatcgactttgcttaatgggtcacaagccaattctattgagaactcaataccgttgtcatcctgcaatcagtgctattgctaatgatctgttttacaaaggagccctcatgaatggtgtaacagaaatagagcggagccctttattggaatggctaccaaccctgtgtttttataatgttaaaggactagaacagatagaaagagataacagctttcataatgtggcagaagctacgtttacactcaagctgattcaatcactgattgcaagtggaatagcaggctctatgattggtgtgataacattatacaaatcccagatgtacaagctttgtcatttactcagtgctgtggactttcaccatcctgatattaaaactgtgcaggtgtccacagtagatgcttttcagggagctgaaaaggagatcattattctgtcctgtgtaaggacaagacaagtaggattcattgattcagaaaaaagaatgaatgttgcattgactagaggaaagaggcatttgttgattgtgggaaatttagcctgtttgaggaaaaatcaactttggggacgagtgatccaacactgcgaaggaagggaagatggattgcaacatgcaaaccagtatgaaccacagctgaaccatctccttaaagattattttgaaaaacaagtggaagaaaaacagaagaaaaagagtgaaaaagagaaatctaaagataaatctcattcataaaaagacatggtgtaaatattttgtatttatgtaaattcagactcattttacatgatatattttttatatttttattactctaaaccctcttattaaaaatatgatatttaaataacatagtaaacacatgtaaaaattttgttcttcaaaaaagtgtacaaaaggtagtataaaatcctactaataaaaataagcttttttctaagaagaatgatttctgtttgccaaagaaatgaattttacaaggggcaggttatagagaatacctgtatacttcaataacaagtgaatgtctccagaactcatctgttggaacatatgtacagaataagatataccaacacctttctaaagtttatcagaatattttttaaatgattataaggcctccctattttataaatgaaaacatattcacaaatatgtttttatgtttaaaacttttgaaaagtacgcaaaagaagaaagaaaaacaccaaaagtttacacctggagatagcagtgttcaacatctgaccatggctagtgccttgcacccatacctgatatagtaggtgcttaataagtatccattggaagaatgaacagatgaatgaaagattacctttggttcattttcttctagtttttttcctctgcatttgtttgcatagtaggtattgatatatatatatatggattacaactttttattataa

SEQ ID NO: 33 - DNA, 499 bp, Homo sapiens
  misc_feature: Incyte ID No 1398040CB1
ctcttcgggaaacggggcccacgtggaccaagggtgactgtgtaaacattatgtccttgaaaatatgttctttttattttttatttttttgagatggagtctcactttgtcgcccaggctggagggcagtggcatgatctcggctcactgcagcctccgcctcctgggttcaagtgatcctcctgcctcaacctcccgagtagctgggattacaggtgtgcaacaccatgcctggctaatttttgtatttttggtagagacggggtttcaccatgttggccaggctggtctccaactcctgacctcaggtgatctgcccgcatcggcctcccaaagtgctcggattacaggtgtgagccactgcgcctggccatctttagtgttttaggggttattttttttagcaatgttatgatggcaattgaaaaaaatacaattcagatcctctctagggtgcagatgtttgaaggaagtgggttcttgcctttcacaaagacaa

SEQ ID NO: 34 - DNA, 712 bp, Homo sapiens
  misc_feature: Incyte ID No 7716061CB1
tgattgcatcactgcactccagcctggacaacagagcaagaccttgtctccaacaaaaaaaaagaaagaaagaaatgtatatatgttttgttgtgcctgagaagttctacatggaaaatagcttcaactgatagcgataaatgtaaaaggccaacttcacttagggccaactgttggtacatactattgtttggtatttccttgttttttgaaatgtggaggctcactctgttacctaggctgcagtgcagtagcacgatctcggctcactacaacctctgcctcctggattcaagtgattctcctgcctcagcctcccgagtagctgggatctcaggcgtgcaccaccacgcccagctaatttttgtatttttagtagagacagggtttcaccttgttggccagactggtgtcgaactcctggcctcaggtgatccacccgccttagcctcccaaagtgctgggattacaggcgtgagccactgtgcctggcaatatttctgagcaatttttgtgagctaaccagttatattttgaatagaattatgcttactattaaatttttccatattttctttttatagagtgagactatctttaaaaaaaaaaaaggccacacatagtgactcatgcctgcaatcccagcactttgggaagccaaggtgggtggatcatgaggtcaggagttcgagaccagcctgaccaatgtggt

SEQ ID NO: 35 - DNA, 1793 bp, Homo sapiens
  misc_feature: Incyte ID No 6113748CB1
ggccggcctcaagatggccgccttctggcgtctccggcgctgttgaatggcgaaagctttattgttcccttcgggcaggagtgttcgtgtcctctatggcgctgtcaataaagaacggcagtttgaatcggtgctgaacagggcctgtcctcccaaagccaactctaaggagaggagaggaagagcagttcttggggcagagttgacgcaatggagctccccaactacagccggcagctgctgcagcagctgtacactctgtgcaaggagcagcagttctgtgattgcaccatctccattggtaccatttacttcagggctcacaagcttgtcctggctgctgccagcctcctgttcaaaaccctgctggataacacagataccatctccatcgatgcatctgtggtgagccccgaggagtttgcgctcttgttggaaatgatgtacacgggcaaactacctgtgggcaagcacaacttctccaaaatcatctccttagcagacagtctacagatgtttgatgtagctgttagctgcaaaaatcttctgaccagccttgtaaactgctcggttcagggtcaggtggtaagggatgtctctgcgccatcctcagagacattcagaaaggaaccagagaagcctcaagtagaaatcctttcatctgaaggtgctggagagcctcattcttccccagagcttgctgccactccagggggccctgtgaaagctgagactgaggaagcagcccattcagtttcacaagagatgagtgtgaattctcccacagcccaggagagccagaggaatgcagaaaccccagcggagactcctactacagctgaagcttgttccccctcccctgctgtgcaaacctttagtgaggcaaagaagacaagcacagaaccaggatgtgaaaggaaacactaccagctgaattttcttctagaaaatgaaggtgtcttctcagatgcactcatggttacccaggatgttttaaaaaaactagaaatgtgttcagaaattaaaggtccacagaaggaggtgattctgaattgctgtgagggcagaacacccaaggagacaatagaaaatttgttgcacagaatgactgaagagaagacgctgactgctgagggtttggtaaaactcctccaggctgtgaagacgactttcccaaacctgggccttctgctagagaagttgcagaaatcagccactttgccaagcaccacagtccaaccaagccctgatgattatgggactgagctattgagacgctatcatgaaaacctctctgagattttcacagacaaccagattttattaaagatgatctcacacatgacaagtttagcccctggagaaagagaggtcatggagaagcttgtgaaacgtgactctggttcaggtggtttcaattctctgatatcagcagttctagaaaagcagactctctctgccacagccatttggcaactgctgctggtggttcaggagacaaagacctgtccattggacctgctcatggaggaaatacgaagggagcctggtgccgatgctttcttccgggcacgtgaccaccccagaacatgccactttagaaacaatcctgaggcataaccagttgatcttggaggccatccaacagaagattgagtgcaagctctttacctcggaggaggagcacctggcagagactgtgaaagagattctgagcattccctctgagacagccagccctgaagcttacctgagagcagtgctgagcagagccatggaaaaatcagtcccgg

SEQ ID NO: 36 - DNA, 858 bp, Homo sapiens
  misc_feature: Incyte ID No 7474037CB1
gcctccctagtgcgggctggcagtgcgggcagagcccggctgagaggggcggccctggaggagacggaggcggcgggtgggcccgaggcgcaagaggaagatgaggacgaagaagaggcgctgccgcactccgaggccatggacgtgttccaggagggtctggctatggtggtgcaggacccgctgctctgcgatctgccgatccaggttactctggaagaagtcaactcccaaatagccctagaatacggccaggcaatgacggtccgagtgtgcaagatggatggagaagtaatgcccgtggttgtagtgcagagtgccacagtcctggacctgaagaaggccatccagagatacgtgcagctcaagcaggagcgtgaagggggcattcagcacatcagctggtcctacgtgtggaggacgtaccatctgacctctgcaggagagaaactcacggaagacagaaagaagctccgagactacggcatccggaatcgagacgaggtttccttcatcaaaaagctgaggcaaaagtgagcctccagacaggacaaccctcttcatcactggtggctgagctttttcccagcaggaatgggtcctcgaatcatcgtgcctctttcacagaaaggacgttgtggtggcctcaccccaggcatgcccaacaggaactgtcagcataaacctgggggccctcaggactaggacagggtgagccagtgctccctcctttcatgtacttggcctgagactgacctctccctaggtccaaatgccctagtcacatggcagacccacggcctggcccactgtataaaataaacctgtttgcttcttagtttgaaaaaaaaaaaaaaa

SEQ ID NO: 37 - DNA, 2387 bp, Homo sapiens
  misc_feature: Incyte ID No 2955646CB1
gagcaaaggggggtgtgtgtgtcaggcacttatcccctgtctgtgctaggagctcggataaacagtcagccgagcctcgacgcccccaaatcgtccgcctccaagccccgcacgcgcggacagctcccgggttgcctgcggcgcaggcgggatgctgcttcgcactagtccagtcctctgccaggcccttcctctcctcgctttcttacgccctttccgctggcatgaattcccctttggcattttctcccctctcccctcttttctgtcatctggtttctctccagtcccccctttgctttctaccttgcgtcgcagggcctgagtcgccctctcgcccagcccccagtcttcagcccagcgtctgtgcttccagtccccactcctccgcgtggtcgtggaggtccaccacctttcttctcaagctcgggaacatgcccttccgccctgcctgcttccttcgcccgtcccgggccgcggaccctgactaatggccggtccctgctgtgtgtggggtgttgtgtttttttcttgtctctccccagcagggcacgggggtgtgaaccagctcgggggggtgtttgtgaacggccggcccctacccgacgtggtgaggcagcgcatcgtggagctggcccaccagggtgtgcggccctgtgacatctcccggcagctgcgggtcagccacggctgtgtcagcaaaatcctgggcaggtactacgagaccggcagcatcaagccgggtgtgatcggtggctccaagcccaaagtggcgacgcccaaagtggtggacaagattgctgaatacaaacgacagaacccgactatgttcgcctgggagattcgagaccggctcctggccgagggcatctgtgacaatgacacagtgcccagcgtctcttccatcaacagaatcatccggaccaaagttcagcagcctttccacccaacgccggatggggctgggacaggagtgaccgcccctggccacaccattgttcccagcacggcctcccctcctgtttccagcgcctccaatgacccagtgggatcctactccatcaatgggatcctggggattcctcgctccaatggtgagaagaggaaacgtgatgaagatgtgtctgagggctcagtccccaatggagattcccagagtggtgtggacagtttgcggaagcacttgcgagctgacaccttcacccagcagcagctggaagctttggatcgggtctttgagcgtccttcctaccctgacgtcttccaggcatcagagcacatcaaatcagaacaggggaacgagtactccctcccagccctgacccctgggcttgatgaagtcaagtcgagtctatctgcatccaccaaccctgagctgggcagcaacgtgtcaggcacacagacatacccagttgtgactggtaagggggcttccaggagggtgggggcactgcgttcagtggagggtgcctcagcccatgccatctgaggcccagtgtgaggagcaggtcccccaccgtgatatttacagagagaacgaggcttctaaaaccagggtgcttcctgaacaggggtgtgcagatgtggggagaaaaaaactggggtcagggcatctgtgggcttcaacctggaaaggctgatgctaggaggggctgttgccagttcttcctcctgtccttcgcctctccctttgtctatttctcttccctctcccaagttgcccagaatcatgagcctcttgttaggatgtctgcagaaagcaaataagccaggctggtgagagtggagcatgggtacccagtgtccagcctccacacttgggtctccaaggctcctgggggacccgcttaccgctccctccaggcagcatgggtgatcatggctttgggcttgagggcatggcacccagctctgtggagtttgagatgagtacaattattccagccttcctcctgcttcccagagaggtcagtgacaccaaggcttgatttcaaggccaggtaggatcaggcttggcccacaatcaaatgcagagctaggggcgccatggccaggagcccctacaaatgaagagcagcagggccagttagtttggaaggggaggtggagtccagggaaggccgcagagctccaggctgtggaatgcacgtgccactgcagaagggtttccaggccaggagactgcccaaatgggagagacacgcattgaggtgttattaaaattcacctaattattctggagaaaaaaaaaaaaaacaaaaaaaaacaaaaacacaacaaaaaaaacacacacacacaagaaaaaaaaaaaaagggggggggcccccaaaatgatggcccaccccccggggaatatccccgggggggcccccagagagggggccaaa

SEQ ID NO: 38 - DNA, 2091 bp, Homo sapiens
  misc_feature: Incyte ID No 1573006CB1
gcggacaggaattctgacgatcgggaaccatcttgtccggccttgatacgtctctgactacgcttcccagaggtctcctcggcaagactgtacttctcgcggtaattcagcttccaactcacgcgctggcgggaccctcagggctttaccagcaactaccccagtgccggggagggttctgctgcttcgaaagctgctctacccttctccaaaagaagagccaagagaaggtccttttctacaaatatcagagccatggctcaggagtcagtgatgttcagtgatgtgtccgtagacttctctcaggaggagtgggaatgcctgaatgatgatcagagagatttatacagagatgtgatgttggagaattacagcaacctggtttcaatggcagggcattctatttctaaaccaaatgtgatctcctacttggagcaagggaaggagccctggttggctgacagagagctaacaagaggccagtggccagtcctggaatcaagatgtgagaccaagaaattatttctgaagaaagaaatttatgaaatagaatcaacccagtgggaaataatggaaaaactcacaagacgtgattttcagtgctccagtttcagagatgattgggaatgtaatcggcagtttaagaaagaactcggctctcaggggggacatttcaatcaattggtattcactcatgaagatctgcccactttgagtcaccatccatccttcacattacagcaaatcattaacagtaaaaagaaattctgtgcatctaaagaatataggaaaacctttagacatggctcacagtttgctacacatgagataattcataccattgagaagccttatgaatgtaaggaatgtggaaagtcctttagacatccctcaagactcactcatcatcagaaaattcatactggcaagaaaccctttgaatgtaaggaatgtggaaaaacctttatttgtggctcagaccttactcgacatcacagaattcacactggtgagaaaccctatgaatgtaaggaatgtgggaaagcctttagtagtggttcaaacttcactcgacatcagagaattcacacagaaaaatggataactatacatttccctgaaatttgcttttttacattcaactgtacattttggattttccttcaatagatgatgatctaacagtctttctgatggttacagtatgttccacagtgttatttaccaatgccttttcaaccactttcccaattgtgatattatggattgtttgcgcttttttgccatctaaaagtaatgctgtaacaaacactttgtattcttgtctgttttatttctataaaataagcttcccaagtcatttccagtgtttttgctttgttttgttttgagacagagtttcactcttgtttcccaggctggagtgcaatggcgcaatcttggcttactgcaatctccgcctcccgggttcaagagattctcctgcctcagcctcccaagtagctggggttacaggtatatgccactacgcctggaaaattttgtatttttagtagagacggcatttttccatgttggtcaggctggtctcgaactcctgacctcaggtgatccaccctcctcggcctcccaaagtgctggcattataggcatgagccaccgcacccggcgtcatttccagtgttttctatgtttcctaggaaggttttgcttctggaagatttcagagggcaagagaataaaggagtggagtgcagtttttcatatatgtgattctgtgagtgtgtatacacagagatttaatattttaatacatcaggaattgattatacgatctaatgcaggatgtcagacttgtttctatccaggaataaaaccagttatgccagaagtaactattattcacctttctcctactgaattaaaatactatcagttgtatattaaatgctcacatctatttctgttctccatattctagcaatccttgacacttttcaatagcttttattacttggaaataaagatgtatgtatatattattaatatatagtttattatttggaaataaagatgtttcttcatgagaaa

SEQ ID NO: 39 - DNA, 2385 bp, Homo sapiens
  misc_feature: Incyte ID No 1336756CB1
tgaaatggcagtgggggggtttcaggagtggggtcccaggaggagctactctgggttaccatgagagagaccttggaggccctcagctccctgggattctctgtgggacagccagagatggccccccaaagtgagcccagggaaggatcccataatgcccaggagcagatgtcctcttctagggaagagagagcactgggggtgtgctcagggcacgaggcccctacaccggaggaaggtgcccacacagaacaagccgaggctccctgcagaggccaggcgtgctcagcacagaaggctcagcctgtgggtacctgcccaggagaggagtggatgattcggaaggtgaaggtggaggacgaagatcaggaggcagaagaggaggtcgaatggccccagcatctatcgttacttcccagcccctttcccgcgcctgacctggggcatctggctgccgcgtacaaactggagccaggggccccgggggcactgagtgggctcgcgctgtctgggtggggtccgatgccggagaagccctacggctgcggggagtgtgagcggcgcttccgggaccagctgacgttgcgactgcaccagcggctgcaccggggcgagggcccctgcgcctgcccggactgcggccgcagcttcacgcagcgcgcccacatgctactgcatcagcgcagccaccgcggcgagcggcctttcccgtgctccgagtgcgacaagcgcttcagcaagaaggcccatctgacccgccacctgcgcacgcacacgggcgagcggccctacccgtgcgcggagtgcggcaagcgcttcagccagaagatccacctgggctcgcaccaaaagacccacaccggcgagcggcccttcccctgcacggaatgcgagaagcgctttcgcaagaagacgcacttgattcggcaccagcgcatccatacgggcgagaggccctaccagtgcgcacagtgcgcacgcagcttcacgcacaagcagcacttggtgcggcaccaaagggtgcaccagacggccggcccggccaggccctctcccgactcgtccgcttctcctcattccactgccccgtccccgaccccatcctttcccgggccaaagcctttcgcctgctccgactgcggcttgagcttcggctggaaaaagaacctcgccacgcaccagtgtctgcaccgcagcgagggtcgcccctttgggtgcgatgagtgcgcactgggcgccaccgtggatgcccccgccgccaagcccctggccagcgcgcctggcggaccgggctgcggcccaggatccgatcccgtggtgccccagcgcgccccctcgggcgagcggtccttcttctgcccggactgcgggcgcggcttctcccatgggcagcacctggcgcggcacccgcgcgtgcacacgggcgaacggcccttcgcctgcacgcagtgtgaccgccgcttcggctcgcggcctaatctggtcgcccactccagggcccacagcggcgccaggcctttcgcctgcgctcagtgcggccgccgcttcagccgcaagtcgcacctgggccgccaccaggcggtgcacactggcagtcgcccccacgcctgcgccgtctgcgcccgcagcttcagctccaaaaccaacctagtccgccaccaggcgatccacacaggctcccgccccttctcctgcccgcagtgcggaaagagcttcagccgcaagacccacctggtgcggcaccagctcattcacggcgaagccgcccacgcggccccggacgccgcccttgcggccccagcctggtccgctccccccgaggtggcgccgcccccgctcttcttctgagcctagttctcacgaggaccctttcttgcccacagtttcgagaggcccgtgccatgagaccgcctggggtgagcaaggcgacctgggctgctgcccgaaggtttggccgccgcgggacacctgtttccttcccgcagtgtctgcgtccgcacagcatacccagctcggacctcctaggacagagactcagcgaacccttgctgggaaccgctgagctgaagttcttggaaggctcccacccaggtgccccgttggaaagcagatatttcccggacccagcgcggcctcaaccagggcaggaaagagtggttatttatgtacttaaagtttcattaaagttaaaatcggacacgttctggggctgctaaatgaattgggggagggaacacctgactctccatgtacgccactcgtcccacctccatccacacacagaccacccccctccacttccccttctgtcctgggtgagttacatttagccagctgctgttaattggttctcgccaaataaatatgtcaatatagttattcccg

SEQ ID NO: 40 - DNA, 1289 bp, Homo sapiens
  misc_feature: Incyte ID No 71259816CB1
ggccggtccccacgccctctcactgcgccctcggtccgccccagatcatccgccagctggagaacaacatcgagaagacaatgatcaagatcatcaccagccagaacatccacctgctgtatttggacctgctggattatctgaagacagtgctggcaggataccccattgagctggacaagctgcagaacctcgtggtcaactactgctcagagctgtcggatatgaagatcatgtcccaagatgccatgatgatcacggatgaggtcaagaggaacatgaggcaaagggaggcgtccttcatcgaggagcgccgggcaagggagaaccggctcaaccagcagaagaagctgatcgacaagatccacacgaaggagaccagcgagaagtaccgccggggccagatggacttggacttcccctcgaacctgatgagcacggagaccctgaaattgaggagaaaagagacctccacagcagaaatggaataccagtcgggcgtgactgctgtggtggagaaggtcaagagtgctgtacggtgctctcacgtctgggacatcactagccgcttcctggcccagaggaacacggaggagaacctggagctgcagatggaggactgtgaggagcggcgggtgcagctgaaggccctggtgaagcagctggagctggaggaggccgtgctcaagttccgccagaagcctagctccatcagcttcaagtccgttgagaagaaaatgacagacatgctaaaagaggaagaagagaggctccagctggcgcacagcaacatgaccaagggccaggagctgctgctgaccatccagatgggcatcgacaacctctatgtccggctgatgggcattaccttgcctgcgacccagcaagctggcgtactgcgaggggaagctcacgtacctggctgacagagtgcagatggtgtccaggaccgaggagggcgacacaaaggtgagggacaccctggagtcctcgactctgatggagaagtacaacaccaggatcagctttgagaaccgggaggaggatatgatcggaggaggcatgccatgccagacgggcccggaggagaggcaccagggctggagcagggaggagcgcgacaccttccagttccccgacatggaccacagctacgtcccttcgcgcgccgagatcaagaggcaggcgcagcggctaatcgaggggaagctcaaggcggccaagaaaaagaagaagtagccccgccgccccgctccctgctttgctacacaaataaacatttttccagg

SEQ ID NO: 41 - DNA, 2628 bp, Homo sapiens
  misc_feature: Incyte ID No 3354130CB1
cgctgaggcgctgtaggtggctccctcccaccacaacgatttcagagagaaacaagtcggaatctgagaagtgaggctccagataaactgtaaactgctggaagggggcgatggctgtggccctgggttgtgcaatccaggcatccttgaatcaaggctctgtgtttcaagaatatgatactgactgtgaagttttccgtcagcgcttcaggcagttccagtacagagaagcagctgggcctcatgaagcatttaacaaactctgggagctttgctgtcaatggctgaagccaaagatgcgctctaaggaacaaatcctggagctgctagtgttggagcaattcctaactatcctgcccacagagatagagacctgggtgagggagcactgcccagagaatagagaaagagttgtgtcactgatagaagacttacagagagaacttgagataccagagcagcaggttgatatgcatgacatgctcttggaagaactggcaccagtgggaacggcacacataccaccaaccatgcacctagagtcacctgcactccaggtaatgggacctgcccaggaggccccagtagcagaggcatggatcccacaggcagggccaccggagctgaactatggtgctactggagaatgtcagaactttctggaccctggatatccattaccaaaacttgacatgaacttctcattggagaatagagaagagccatgggtgaaggaattacaggattctaaagaaatgaaacaattacttgattccaagataggttttgagatcgggatagaaaatgaagaagatacttcaaaacagaaaaaaatggagactatgtatccatttattgtaactttagaggggaatgctctccagggtcccattttgcaaaaagactatgtacagttagaaaatcaatgggaaacccccccagaggatttacagacagatttagcaaaactggtagatcagcagaaccccactctgggagagacacctgagaactccaacttggaagaacctctcaaccctaaaccccacaagaaaaagagtccaggagagaaacctcaccgatgtcctcagtgtggaaaatgttttgctcggaagtcacaacttactgggcatcagagaattcattcaggagaagaacctcacaaatgccctgaatgtgggaaaagattccttcgtagttcagacctttatagacaccaacgacttcatacaggggagagaccctatgaatgcactgtatgtaaaaagcgattcactcggcggtcacatcttatagggcaccagagaacccattctgaagaagaaacatataaatgtcttgagtgtgggaaaagtttttgtcatggatcaagtcttaaaagacatctgaaaactcatacaggtgaaaaacctcatagatgtcataattgtgggaaaagttttagtcgactgacagctcttactttgcaccagagaacgcatactgaagagagaccttttaaatgtaattattgtgggaaaagttttagacagagaccaagcctcgttattcatttaagaatccacacaggggagaagccatacaagtgtactcattgttctaaaagcttcagacagagagccggccttattatgcaccaggtcactcactttagaggacttatttaagaattgctaagggaaacaggtcttacacaaattgacactaactcaaaaaaaatcttaacctgcagcaggctgtttgtcttggaagcttttgtttgagcttataagaacatagacagcttttttttttactagttttaaaacccatcttccaaggtatatgaattctagagtatttatctactcctgtgattttcttagatttgatttcttctgtctgcacaactcttctttttttaattacaatgaaaaattttgtgttccaaggcaactgtatcataggtgtaaacataaagcatataaattatgacaatccttttagaggtagggtcaatatagtggataaacctgtctatcagacgtattgattatagcagtactatagttattctgctgtcattattaaagatgattatattcattcaaagctttagatgtgtcccatgtggcaagaaaggagacagtgaattttgtcaaacaataaaaatgtgtcaggaacacaaggatgaaggggatgtcatttgcctggtaagaactgggttatttccactgaaatttgttatgtttaaggaaattaagattttaagcttgaaattatacaagcagaatctaatttaattttgattgactgaagaaccaggtcttttgctctcctttggtatttcactctcctttggtattcaatcatgtgtcttttagtgctttttaaaattttacccattctttaattcagcatctccctatgtattgtgtcatagaatacttagttctgctagatattctgcaatataattcgggaattacttccctgttgtttccgcttcttagggttcatcagtaccacattcagaattatgagaatctcatgagaggtctctagaagcgagcaaaaagccctctttgggtcatgttttccaaattatttgacccaaaccttttatattccataatagtaagaatattc

SEQ ID NO: 42 - DNA, 4077 bp, Homo sapiens
  misc_feature: Incyte ID No 1797985CB1
aaatgcccaaagtaccatatttaaaagctctcattttaggataatcattgagttattctatagatgattcatgtaactatcttacatgattatgtatctcaaccttctctttaaaataatacctcataaagcttgaatggaggtttttatgattgtaggcgcctgatgatagagaagattgctgcccatctcgcggatttcacacctcgtcttcagagtaacacaagagcactttatcagtattgccccattcctataatcaactatccacaactcgaaaatgaactattttgtaatatttattacctcaaacaactgtgtgatacactccggtttccagattggccaattaaagacccggttaagcttctaaaagatacccttgatgcctggaagaaagaagtagaaaagaagccacctatgatgtcaatagatgatgcttatgaagtgcttaatctgcctcaaggacagggaccgcatgatgagagcaagattaggaaagcttacttcagacttgcacaaaagtaccaccctgataagaatccagaagggagggacatgtttgaaaaagtaaataaagcatatgaatttttatgtaccaaatcagcaaaaatagtggatgggccagatccagagaatataattttaattctaaaaacacagagcatcctcttcaaccgtcataaagaagatttacagccttataaatatgcaggataccccatgcttattcggactataacaatggaaacttcagatgacctccttttctcaaaagaatcaccattgttgcctgcggctacagagctagctttccatactgtcaactgttcagccctcaatgctgaagagctcagaagagagaatggactagaggtgttacaagaggcatttagtcgctgtgtggctgtcttgactcgttctagtaaaccaagtgacatgtcagtacaggtgtgtggatacataagtaaatgctacagtgtggctgctcagtttgaggaatgccgagagaagatcacggaaatgcctagcatcatcaaggatctctgtcgggtactatattttggcaagagtattccccgcgtagctgctcttggggtagaatgtgtcagttcttttgctgtggatttctggctacagacacacctatttcaggctggaattttgtggtatctccttggttttctgtttaattatgactacacactagaagagagtggcattcagaaaagtgaagaaacaaaccagcaggaggtagcaaacagccttgccaaactgagtgtccatgctctgagtcgccttggagggtatttggctgaagaacaagcaactccagaaaatccaaccataaggaaaagcttagctggcatgctgacaccctatgttgctagaaaacttgctgtggctagtgtgactgagattttgaagatgcttaacagcaacacagaaagtccatatttgatatggaacaattctacaagagcagaattacttgaatttcttgaatcccaacaagaaaacatgattaaaaaaggtgattgtgacaaaacttatggatcagaatttgtctacagtgatcatgccaaagaacttattgtaggggagatttttgttagggtgtataatgaagttcctactttccaactggaggttccaaaagcatttgctgcaagtctcttggattatataggctcgcaggcccaatacttgcacacattcatggccatcacacacgcggcaaaagtggagtcagagcaacatggagatcgcttaccgagagtagaaatggctttggaggctctgagaaatgtcataaaatacaatccaggttctgagagtgaatgcattgggcactttaagttgatattttctcttctccgagttcatggagctggtcaagtgcagcagttggctttagaggttgtgaatatagtgacatctaaccaagactgtgtcaacaatattgctgaatcaatggttttgtccagtttattggctcttctacattcattgccatcaagtcgtcagcttgttctggaaactctttatgctttgacatcgagtacaaaaataatcaaagaagcaatggcaaagggtgctttgatctatttactggatatgttctgcaattcaacacatccacaggttcgagcccaaacagcagaactttttgccaaaatgacagcagataaactgataggtccaaaggttcgaattacgttaatgaaatttctaccaagcgttttcatggatgctatgagagacaatcctgaagctgctgtacatatttttgaaggaactcatgaaaatcctgagttaatttggaatgataattccagagataaagtgtccacaacagttagggaaatgatgctagagcactttaaaaatcagcaggacaaccctgaggcaaactggaagttgcctgaagattttgctgtggtgtttggagaagcagagggtgaacttgctgttggaggagtcttcttgaggatctttattgcacaaccagcctgggttctaagaaagcctagagaatttcttattgccctgttagaaaaattaactgagctcctagagaagaacaatcctcatggagaaactctggaaaccttgacaatggcaacagtgtgtctcttcagcgcacaacctcagctggcagatcaggtcccgccattgggccatcttcccaaagttatccaggcaatgaatcataggaacaatgccattcctaagagtgccattcgggttatccatgccttgtctgaaaatgagctgtgtgttcgagccatggcatctttagagaccattggcccactgatgaatggaatgaaaaagcgagcagatactgttggtctagcctgtgaagcaattaatcgaatgtttcagaaggagcagagtgaattagtagcacaagccctgaaagcagatttggttccatacctcttaaaattactcgaaggcattggccttgaaaacctggacagcccagcagccactaaggctcagattgttaaagctctcaaggcaatgactcgaagtttgcagtatggagaacaggtgaatgaaatcctgtgccgttcttcagtctggagtgccttcaaagatcagaaacatgatttgttcatttctgagtcacaaacagcaggatacctcacaggacctggagttgctggctaccttaccgcaggtacatctacatcagtcatgtctaacctgccacctcctgtagaccatgaggcaggcgaccttggctatcagacttgaaatattcacgagagacaataaacgctgaaaggccagtgccaagtccacattcctccagctgatacgttgaagcaaactcttactgcctttctcctggtttcatgacagtgttattcctttttctataaatatatttttaggaaaaaaagtcagtgatcctaattgtatcacattataagaaagcactctgtggatcaacataagtgggtacacaagaattttttttttcttggtgtatgtaagcacatttgttcctttatatctgtttacaaaactgtgaatcaaaaagacaaaactttcttcctagtttttgtaattttttttttgaactagcatgactgtagggttgagctacagtcaacaaaaattgggctaagtcacttttccccaggaaagaatatttccctctcctgcatcaagtctgcgtggccatcctccccccaccatccaagactattaggttttgtccctgcacccttcactggcatcctcaatcattaaccttctgaaagctcacagtacacattagtatgtataactggctttaccaaattgaatgaaaaggagcttgtgcaaaaaaatttaaaaatggatgtcaagatgttatgtaaaagatgagtataattgtgaaatgttctatacactatcaaatatataaagctttctatattgaatgtacattatacagatcattcatatgtgtacataaaattttaaaaataaagggaattgactgctttgttaatgagatatatttgttctagtttaatctttccgtttgaagacctcatatatctatctttatttcta

SEQ ID NO: 43 - DNA, 1570 bp, Homo sapiens
  misc_feature: Incyte ID No 2870383CB1
atgcaatcccggcttctactcctcggggcacccggaggccacggcggcccggcctcgcggcgcatgcggctgctcctgcggcaggtggtgcagcgcaggccgggtggcgacaggcagcggccggaggtcagactgttgcacgccggctcgggggccgacacaggtgatacagttaatattggagatgtatcctacaagttgaaaattcctaagaatccagaacttgtgccacagaactacatttcagactctctggctcaatctgtagttcagcatctaagatggataatgcagaaggatcttttggggcaagatgtttttctaataggacctcctgggcctcttcgacgctctattgctatgcagtacttggagctgaccaaacgggaggtcgaatacattgccctgtcaagggacaccactgaaactgatctcaaacagcgacgagagatccgtgcaggcacagccttttacattgatcagtgtgcagttcatgcagccacagaaggcagaactctcattttggaaggtttggaaaaggcagagaggaatgttttgcctgttttgaacaacttgctggaaaacagagagatgcagcttgaagatggacgcttcctgatgtctgctgagcgttacgacaaacttctccgagatcataccaaaaaagagttggattcttgggaaattgtccgagttagtgaaaatttccgagtgattgccttgggcttgccagtgccaaggtattctgggaatccattagacccccctcttcgttctcgatttcaagccagggatatttattatttacccttcaaggaccaacttaagttgttatattcaattggagccaatgtttctgctgagaaagtttctcagctcttgtcctttgccacaactctgtgttcccaagaatcttctactcttggacttccagactttcctttagatagtttagcagctgcggttcaaatcttggattcctttcctatgatgccaatcaaacatgcaatccagtggctttatccatatagtattttactaggtcatgaagggaagatggctgtggaaggtgttttaaagcgctttgaacttcaagattcaggaagctctctacttcctaaagagattgtaaaagtagagaagatgatggaaaaccatgtgtcccaagcttctgtgaccatccggattgcagataaagaggtgaccattaaggtgccagccgggaccaggctattaagtcaaccttgtgcgtcagaccgtttcatacagactttgagccataagcagctacaggctgaaatgatgcagtctcacatggttaaagatatatgtttaattggaggaaagggttgtggaaaaacagtgatcgctaagaactttgccgataccttaggatacaacatagaacctattatgctctatcaggtacagtgttcatttttagctgcacttggactataagcatatatgcttaggccatcagcagaatttatggatatcttcttgttatgacctgtgtttttttatataggtaaagaaccaaaatagtaataaaattattcaaattaaa

SEQ ID NO: 44 - DNA, 2642 bp, Homo sapiens
  misc_feature: Incyte ID No 1285088CB1
gcacgagggtgagggcggcgatgagagcgaaagttgcgctcggctcgtcgctgggggcttgaagcggctccgcgctctgcccgtttgggcctcccccgactcggactcgcgcccgtgggctcccgccgcgcccgcccggccccgcgccggccccgcgccccctcccccgtctcggcgccccctcctcaggagccgcgggtccccgccactttcgcacggccccggcccccaccgatgccggccatggtggagaagggccccgaggtctcagggaagcggagagggaggaacaacgcggccgcctccgcctccgccgccgccgcctccgccgccgcctcggccgcctgcgcctcgccagccgccactgccgcctcgggcgccgccgcctcctcagcctcggccgccgccgcctcagccgccgccgcccccaataatggccagaataaaagtttggcggcggcggcgcccaatggcaacagcagcagcaactcctgggaggaaggcagctcgggctcgtccagcgacgaggagcacggtggcggtggcatgagggtcggaccccagtaccaggcggtggtgcccgacttcgaccccgccaaactggcaagacgcagtcaagaacgggacaatcttggcatgttggtctggtcacccaatcaaaatctgtcagaagcaaagttggatgaatacattgccattgccaaagaaaagcatgggtacaacatggaacaggctcttgggatgctcttctggcataaacataatatcgaaaagtcattggctgatttgcccaactttacccctttcccagatgagtggactgtggaagataaagtcttatttgagcaagcctttagttttcatgggaaaacttttcatagaatccaacaaatgcttccagataaatctatagcaagtctggtgaaattttactattcttggaagaagacgaggactaaaactagtgtgatggatcgccatgcccggaaacaaaaacgggagcgggaggagagcgaggatgaactggaagaggcaaatggaaacaatcccattgacattgaggttgatcaaaacaaggaaagcaaaaaggaggttccccctactgagacagttcctcaggtcaaaaaagaaaaacatagcacacaagctaaaaatagagcaaaaaggaaacctccaaaaggaatgtttctttctcaagaagatgtggaggctgtttctgccaatgccactgctgctaccacggtgctgagacaactagacatggaattggtttcagtcaaacgacagatccagaatattaaacagacaaacagtgctctcaaagaaaaacttgatggtggaatagaaccatatcgacttccagaggtcattcagaaatgtaatgcacgttggactacagaagagcagcttctcgccgtacaagccatcaggaaatatggccgagattttcaggcaatctcagacgtgattgggaacaaatcagtggtacaagtgaaaaacttttttgtaaattatcgacgccgcttcaacatagatgaagttttacaagaatgggaggcagaacatggtaaagaagagaccaatgggcccagtaaccagaagcctgtgaagtccccagataattccattaagatgcccgaagaggaagacgaggctcctgttctggatgtcagatatgcatctgcctcctgagaaactggtggctttgaacacttggtgtggactactgtgttatccgggatatcaggtattatgagacatcacctagccatctgcatcacatctctctggacaagcagctattaccaaaaaaggcatatacttccagtcctgtgctccatctgccttaattctttgctcgttcctccatgttggcgccacttcccagagagctccactgcatctcacactctgcccacgtgctggggaagtctcacggcctgcacatctcttgtgactctgggaaccgcctctcccgccggagcccccgagccccaccaatggcagctcttcccagtcagcagcttcagagcaggcagtctccttggaaggcccgactctgttcctgcatggcctgcagtttctactttgtgcatagagtcattttcagagtcaccgcgaccctgtggccttctagaaagtttcttttgttcttttctgagacaaccacctaagtgataatacgcttttttggaaactaatatatattgccagactgcatcataacctttatcatgccaagcatcctgatgcaactcacatttccctaaacatggggtacagttatgatttataaattgagttggcttaaatctccctcttctcccttcccaagtgttacaaagatcatttactgcaactgtcgttggacactgtagcttaaagggaacgtggacctcaatgctttctgccttcaacttttcagcattgtgaccccagggtggttgccaccccatcttttcctgaccccccccacccccccacctccaagaggttcggcccacatcactgtacctggtgcttgtaaatttggaattggtgccttctccttttggcaaccatggttatcaatcctttttctgttttagtgtcttatttctcctttcaagttatttgcttagccaaagatgacatcactgagg

SEQ ID NO: 45 - DNA, 2618 bp, Homo sapiens
  misc_feature: Incyte ID No 1532441CB1
gcgagtcggtgtgtcctggctgggacggaagttgcaccacaagtacaaattagtttcaggtttgtttcttttccaggcacccagcaacggcggcctccaggcctcaggccccctcaccatcctagaggtcaagatcagctctggctagttctcacaggtctgacccaacaagtagcactgacatttttacgtttgctggatgtacacacggaagtggaggaggaggaggagaaggaggagggcagctccttagctcaagagcaagtggcccaaggcctcagaagactagaaggaagttcctggccattcagcatggtttcccacgggtcctcgccctccctcctggaggccctgagcagcgacttcctggcctgtaaaatctgcctggagcagctgcgggcacccaagacactgccctgcctgcatacctactgccaagactgcctggcacagctggcggatggcggccgcgtccgctgccccgagtgccgcgagacagtgcctgtgccgcccgagggtgtggcctccttcaagaccaacttcttcgtcaatgggctgctggacctggtgaaggcccgggcctgtggagacctgcgtgccgggaagccagcctgtgccctgtgtcccctggtgggtggcaccagcaccggggggccggccacggcccggtgcctggactgtgccgatgacttgtgccaggcctgtgccgacgggcaccgctgcacccgccagacccacacccaccgcgtggtggacctggtgggctacagggccgggtggtatgatgaggaggcccgggagcgccaagcggcccagtgtccccagcaccccggggaggcactgcgcttcctgtgccagccctgctcacagttgctgtgcagagagtgccgcctagacccccacctggaccacccctgcctgcctctggctgaagctgtgcgtgcccggaggccgggcctggagggactgctggccggtgtggacaataacctggtggagctggaggcagcgcggagggtggagaaggaggcgctagcccggctgcgggagcaggcggcccgggtggggactcaggtggaggaggcggctgagggcgtcctccgggccctgctggcccagaagcaggaggtgctggggcagctacgagcccacgtggaggctgccgaagaagctgctcgggagaggctggcggagcttgagggccgggagcaggtggccagggcggcagccgccttcgcccgccgggtactcagcctggggcgagaggccgagatcctctccctggaaggggcgatcgcacagcggctcaggcagctgcagggctgcccctgggcaccaggcccggccccctgcctgctcccacagctggagctccatcctgggctcctggacaagaactgccaccttcttcggctgtcctttgaggagcagcagccccagaaggatggtgggaaagacggagctggtacccagggaggtgaggagagccagagccggagggaggatgagccgaagactgagagacagggtggagtccagccccaggccggagatggagcccagaccccaaaagaggaaaaagcccagacaacccgagaagagggagcccagaccttggaggaggacagggcccagacaccccacgaggatggaggaccccagccccacaggggtggcagacccaacaagaagaaaaagttcaaaggcaggctcaagtcaatttcccgggagcccagcccagccctggggccgaatctggacggctctggcctcctccccagacccatcttttactgcagtttccccacgcggatgcctggagacaagcggtccccccggatcaccgggctctgtcccttcggtccccgggagatcctggtggcggatgagcagaaccgggcactgaaacgcttctccctcaacggcgactacaagggcaccgtgccggtccctgagggctgctccccttgcagcgtggccgccctgcagagcgcggtggccttctccgctagcgcacggctctatctcatcaaccccaacggcgaagtgcagtggcgcagggccctgagcctctcccaggccagccacgcggtggcggcactgcctagcggggaccgcgtggctgtcagcgtggcgggccacgtggaggtgtacaatatggaaggcagcctggccacccggttcattcctggaggcaaggccagccggggcctgcgggcgctggtgtttctgaccaccagcccccaggggcatttcgtggggtcggactggcagcagaatagtgtggtaatctgtgatgggctgggccaggtggttggggagtacaaggggccaggcctgcatggctgccagccgggctccgtgtctgtggataagaagggctacatctttctgacccttcgagaagtcaacaaggtggtgatcctggacccgaaggggtccctccttggagacttcctgacagcctaccacggcctggaaaagccccgggttaccaccatggtggatggcaggacatcatcaaagtccgggtggacacattccattatctacaaattacaaaggtaggcacagcaaagaataatgaagattataagaaaaccaagcgccaggcagcc

SEQ ID NO: 46 - DNA, 6294 bp, Homo sapiens
  misc_feature: Incyte ID No 3056408CB1
caacaaaggagtcacccggcgatgagccccggcacccccggaccgaccatggcagatcccaggcagcccaatggatccaatggtgatgaagagacctcagttgtatggcatgggcagtaaccctcattctcagcctcagcagagcagtccgtacccaggaggttcctatggccctccaggcccacagcggtatccaattggcatccagggtcggactcccggggccatggccggaatgcagtaccctcagcagcagatgccacctcagtatggacagcaaggtgtgagtggttactgccagcagggccaacagccatattacagccagcagccgcagcccccgcacctcccaccccaggcgcagtatctgccgtcccagtcccagcagaggtaccagccgcagcaggacatgtctcaggaaggctatggaactagatctcaacctcctctggcccccggaaaacctaaccatgaagacttgaacttaatacagcaagaaagaccatcaagtttaccagttgaagtcttggcctcggaggatgcagcctttggactcaaggatctgtctggctccattgatgacctccccacgggaacggaagcaactttgagctcagcagtcagtgcatccgggtccacgagcagccaaggggatcagagcaacccggcgcagtcgcctttctccccacatgcgtcccctcatctctccagcatcccggggggcccatctccctctcctgttggctctcctgtaggaagcaaccagtctcgatctggcccaatctctcctgcaagtatcccaggtagtcagatgcctccgcagccacccgggagccagtcagaatccagttcccatcccgccttgagccagtcaccaatgccacaggaaagaggttttatggcaggcacacaaagaaaccctcagatggctcagtatggacctcaacagacaggaccatccatgtcgcctcatccttctcctgggggccagatgcatgctggaatcagtagctttcagcagagtaactcaagtgggacttacggtccacagatgagccagtatggaccacaaggtaactactccagacccccagcgtatagtggggtgcccagtgcaagctacagcggcccagggcccggtatgggtatcagtgccaacaaccagatgcatggacaagggccaagccagccatgtggtgctgtgcccctgggacgaatgccatcagctgggatgcagaacagaccatttcctggaaatatgagcagcatgacccccagttctcctggcatgtctcagcagggagggccaggaatggggccgccaatgccaactgtgaaccgtaaggcacaggaggcagccgcagcagtgatgcaggctgctgcgaactcagcacaaagcaggcaaggcagtttccccggcatgaaccagagtggacttatggcttccagctctccctacagccagcccatgaacaacagctctagcctgatgaacacgcaggcgccgccctacagcatggcgcccgccatggtgaacagctcggcagcatctgtgggtcttgcagatatgatgtctcctggtgaatccaaactgcccctgcctctcaaagcagacggcaaagaagaaggcactccacagcccgagagcaagtcaaaggatagctacagctctcagggtatttctcagcccccaaccccaggcaacctgccagtcccttccccaatgtcccccagctctgctagcatctcctcatttcatggagatgaaagtgatagcattagcagcccaggctggccaaagactccatcaagccctaagtccagctcctccaccactactggggagaagatcacgaaggtgtacgagctggggaatgagccagagagaaagctctgggtcgaccgatacctcaccttcatggaagagagaggctctcctgtctcaagtctgcctgccgtgggcaagaagcccctggacctgttccgactctacgtctgcgtcaaagagatcgggggtttggcccaggttaataaaaacaagaagtggcgtgagctggcaaccaacctaaacgttggcacctcaagcagtgcagcgagctccctgaaaaagcagtatattcagtacctgtttgcctttgagtgcaagatcgaacgtggggaggagcccccgccggaagtcttcagcaccggggacaccaaaaagcagcccaagctccagccgccatctcctgctaactcgggatccttgcaaggcccacagaccccccagtcaactggcagcaattccatggcagaggttccaggtgacctgaagccacctaccccagcctccacccctcacggccagatgactccaatgcaaggtggaagaagcagtacaatcagtgtgcacgacccattctcagatgtgagtgattcatccttcccgaaacggaactccatgactccaaacgccccctaccagcagggcatgagcatgcccgatgtgatgggcaggatgccctatgagcccaacaaggacccctttgggggaatgagaaaagtgcctggaagcagcgagccctttatgacgcaaggacagatgcccaacagcagcatgcaggacatgtacaaccaaagtccctccggagcaatgtctaacctgggcatggggcagcgccagcagtttccctatggagccagttacgaccgaaggcatgaaccttatgggcagcagtatccaggccaaggccctccctcgggacagccgccgtatggagggcaccagcccggcctgtacccacagcagccgaattacaaacgccatatggacggcatgtacgggcccccagccaagcgccacgagggcgacatgtacaacatgcagtacagcagccagcagcaggagatgtacaaccagtatggaggctcctactcgggcccggaccgcaggcccatccagggccagtacccgtatccctacagcagggagaggatgcagggcccggggcagatccagacacacggaatcccgcctcagatgatgggcggcccgctgcagtcgtcctccagtgaggggcctcagcagaatatgtgggcagcacgcaatgatatgccttatccctaccagaacaggcagggccctggcggccctacacaggcgcccccttacccaggcatgaaccgcacagacgatatgatggtacccgatcagaggataaatcatgagagccagtggccttctcacgtcagccagcgtcagccttatatgtcgtcctcagcctccatgcagcccatcacacgcccaccacagccgtcctaccagacgccaccgtcactgccaaatcacatctccagggcgcccagcccagcgtccttccagcgctccctggagaaccgcatgtctccaagcaagtctccttttctgccgtctatgaagatgcagaaggtcatgcccacggtccccacatcccaggtcaccgggccaccaccccaagcacccccaatcagaagggagatcacctttcctcctggctcagtagaagcatcacaaccagtcttgaaacaaaggcgaaagattacctccaaagatatcgttactcctgaggcgtggcgtgtgatgatgtcccttaaatcaggtcttttggctgagagtacgtgggctttggacactattaatattcttctgtatgatgacagcactgttgctactttcaatctctcccagttgtctggatttctcgaacttttagtcgagtactttagaaaatgcctgattgacatttttggaattcttatggaatatgaagtgggagaccccagccaaaaagcacttgatcacaacgcagcaaggaaggatgacagccagtccttggcagacgattctgggaaagaggaggaagatgctgaatgtattgatgacgacgaggaagacgaggaggatgaggaggaagacagcgagaagacagaaagcgatgaaaagagcagcatcgctctgactgccccggacgccgctgcagacccaaaggagaagcccaagcaagccagtaagttcgacaagctgccaataaagatagtcaaaaagaacaacctgtttgttgttgaccgatctgacaagttggggcgtgtgcaggagttcaatagtggccttctgcactggcagctcggcgggggtgacaccaccgagcacattcagactcactttgagagcaagatggaaattcctcctcgcaggcgcccacctccccccttaagctccgcaggtagaaagaaagagcaagaaggcaaaggcgactctgaagagcagcaagagaaaagcatcatagcaaccatcgatgacgtcctctctgctcggccaggggcattgcctgaagacgcaaaccctgggccccagaccgaaagcagtaagtttccctttggtatccagcaagccaaaagtcaccggaacatcaagctgctggaggacgagcccaggagccgagacgagactcctctgtgtaccatcgcgcactggcaggactcgctggctaagcgatgcatctgtgtgtccaatattgtccgtagcttgtcattcgtgcctggcaatgatgccgaaatgtccaaacatccaggcctggtgctgatcctggggaagctgattcttcttcaccacgagcatccagagagaaagcgagcaccgcagacctatgagaaagaggaggatgaggacaagggggtggcctgcagcaaagatgagtggtggtgggactgcctcgaggtcttgagggataacacgttggtcacgttggccaacatttccgggcagctagacttgtctgcttacacggaaagcatctgcttgccaattttggatggcttgctgcactggatggtgtgcccgtctgcagaggcacaagatccctttccaactgtgggacccaactcggtcctgtcgcctcagagacttgtgctggagaccctctgtaaactcagtatccaggacaataatgtggacctgatcttggccactcctccatttagtcgtcaggagaaattctatgctacattagttaggtacgttggggatcgcaaaaacccagtctgtcgagaaatgtccatggcgcttttatcgaaccttgcccaaggggacgcactagcagcaagggccatagctgtgcagaaaggaagcattggaaacttgataagcttcctagaggatggggtcacgatggcccagtaccagcagagccagcacaacctcatgcacatgcagcccccgcccctggaaccacctagcgtagacatgatgtgcagggcggccaaggctttgctagccatggccagagtggacgaaaaccgctcggaattccttttgcacgagggccggttgctggatatctcgatatcagctgtcctgaactctctggttgcatctgtcatctgtgatgtactgtttcagattgggcagttatgacataagtgagaaggcaagcatgtgtgagtgaagattagagggtcacatataactggctgttttctgttcttgtttatccagcgtaggaagaaggaaaagaaaatctttgctcctctgccccattcactatttaccaattgggaattaaagaaataattaatttgaacagttatgaaattaatatttgctgtctgtgtgtataagtacatcctttggggttttttttttctcttttttttaaccaaagttgctgtctagtgcattcaaaggtcactttttgttcttcacagatctttttaatgttctttcccatgttgtattgcatttttgggggaagcaaattgactttaaagaaaaaagttgtggcaaaagatgctaagatgcgaaaatttcaccacactgagtcaaaaaggtgaaaaattatccatttcctatgcgttttactcctcagagaatgaaaaaaactgcatcccatcacccaaagttctgtgcaatagaaatttctacagatacaggtataggggctcaaggaggtatgtcggtcagtagtcaaaactatgaaatgatactggtttctccacaggaatatggttccattaggctgggagcaaaaacaatgttttttaagattgagaatacatacctgacaacgatccggaaactgctcctcaccactcccgtcatgcctgctgtcggcgtttgaccttccacgtgacagttcttcacaattcctttcatcattttttaaatattttttttactgcctatgggctgtgatgtatatagaagttgtacattaaacataccctcatttttttcttttcttttttttttttttttttagtacaaagttttagtttctt

(19) United States
(12) Patent Application Publication
(10) Pub. No.: US 2004/0152093 A1
(43) Pub. Date: Aug. 5, 2004

(54) Nucleic acid-associated proteins

(76) Inventors: Yue Henry, Sunnyvale, CA (US); Li Ding, China (CN); Mariah R Baughn, Los Angeles, CA (US); Preeti G Lal, Santa Clara, CA (US); Huibin Yue, Cupertino, CA (US); April J A Hafalia, Daly City, CA (US); Ernestine A Lee, Kensington, CA (US); Craig H Ison, San Jose, CA (US); Shanya D Becha, San Francisco, CA (US); Rajagopal Gururajan, San Jose, CA (US); Brooke M Emerling, Chicago, IL (US); Jennifer A Griffin, Fremont, CA (US); Y Tom Tang, San Jose, CA (US); Dyung Aina M Lu, San Jose, CA (US); Monique G Yao, Mountain View, CA (US); Narinder K Chawla, Union City, CA (US); Jayalaxmi Ramkumar, Fremont, CA (US); Ameena R Gandhi, San Francisco, CA (US); Soo Yeun Lee, Mountain View, CA (US); Richardson W Thomas, Redwood City, CA (US); Junming Yang, San Jose, CA (US); Vicki S Elliott, San Jose, CA (US); Yan Lu, Mountain View, CA (US); Kavitha Thangavelu, Sunnyvale, CA (US); Ann He, San Jose, CA (US); Yalda Azimzai, Oakland, CA (US); Brigitte E Raumann, Chicago, IL (US); Anita Swarnakar, San Francisco, CA (US); Neil Burford, Durham, CT (US)

Correspondence Address: FOLEY AND LARDNER, SUITE 500, 3000 K STREET NW, WASHINGTON, DC, 20007, US

(21) Appl. No.: 10/476,924

(22) PCT Filed: May 2, 2002
(86) PCT No.: PCT/US02/14276

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

The invention provides human nucleic acid-associated proteins (NAAP) and polynucleotides which identify and encode NAAP. The invention also provides expression vectors, host cells, antibodies, agonists, and antagonist. The invention also provides methods for diagnosing, treating, or preventing disorders associated with aberrant expression of NAAP.

### TECHNICAL FIELD

This invention relates to nucleic acid and amino acid sequences of nucleic acid-associated proteins and to the use of these sequences in the diagnosis, treatment, and prevention of cell proliferative, neurological, developmental, and autoimmune/inflammatory disorders, and infections, and in the assessment of the effects of exogenous compounds on the expression of nucleic acid and amino acid sequences of nucleic acid-associated proteins.

### BACKGROUND OF THE INVENTION

Multicellular organisms are comprised of diverse cell types that differ dramatically both in structure and function. The identity of a cell is determined by its characteristic pattern of gene expression, and different cell types express overlapping but distinctive sets of genes throughout development. Spatial and temporal regulation of gene expression is critical for the control of cell proliferation, cell differentiation, apoptosis, and other processes that contribute to organismal development. Furthermore, gene expression is regulated in response to extracellular signals that mediate cell-cell communication and coordinate the activities of different cell types. Appropriate gene regulation also ensures that cells function efficiently by expressing only those genes whose functions are required at a given time.

Transcription Factors

Transcriptional regulatory proteins are essential for the control of gene expression. Some of these proteins function as transcription factors that initiate, activate, repress, or terminate gene transcription. Transcription factors generally bind to the promoter, enhancer, and upstream regulatory regions of a gene in a sequence-specific manner, although some factors bind regulatory elements within or downstream of a gene coding region. Transcription factors may bind to a specific region of DNA singly or as a complex with other accessory factors. (Reviewed in Lewin, B. (1990) Genes IV, Oxford University Press, New York, N.Y., and Cell Press, Cambridge, Mass., pp. 554-570.)

The double helix structure and repeated sequences of DNA create topological and chemical features which can be recognized by transcription factors. These features are hydrogen bond donor and acceptor groups, hydrophobic patches, major and minor grooves, and regular, repeated stretches of sequence which induce distinct bends in the helix. Typically, transcription factors recognize specific DNA sequence motifs of about 20 nucleotides in length. Multiple, adjacent transcription factor-binding motifs may be required for gene regulation.

Many transcription factors incorporate DNA-binding structural motifs which comprise either a helices or B sheets that bind to the major groove of DNA. Four well-characterized structural motifs are helix-turn-helix, zinc finger, leucine zipper, and helix-loop-helix. Proteins containing these motifs may act alone as monomers, or they may form homo- or heterodimers that interact with DNA.

The helix-turn-helix motif consists of two a helices connected at a fixed angle by a short chain of amino acids. One of the helices binds to the major groove. Helix-turn-helix motifs are exemplified by the homeobox motif which is present in homeodomain proteins. These proteins are critical for specifying the anterior-posterior body axis during development and are conserved throughout the animal kingdom. The Antennapedia and Ultrabithorax proteins of Drosophila melanogaster are prototypical homeodomain proteins. (Pabo, C. O. and R. T. Sauer (1992) Annu. Rev. Biochem. 61:1053-1095.) The zinc finger motif, which binds zinc ions, generally contains tandem repeats of about 30 amino acids consisting of periodically spaced cysteine and histidine residues. Examples of this sequence pattern, designated C2H2 and C3HC4 (“RING” finger), have been described. (Lewin, supra.) Zinc finger proteins each contain an α helix and an antiparallel B sheet whose proximity and conformation are maintained by the zinc ion. Contact with DNA is made by the arginine preceding the α helix and by the second, third, and sixth residues of the α helix. Variants of the zinc finger motif include poorly defined cysteine-rich motifs which bind zinc or other metal ions. These motifs may not contain histidine residues and are generally nonrepetitive. The zinc finger motif may be repeated in a tandem array within a protein, such that the α helix of each zinc finger in the protein makes contact with the major groove of the DNA double helix. This repeated contact between the protein and the DNA produces a strong and specific DNA-protein interaction. The strength and specificity of the interaction can be regulated by the number of zinc finger motifs within the protein. Though originally identified in DNA-binding proteins as regions that interact directly with DNA, zinc fingers occur in a variety of proteins that do not bind DNA (Lodish, H. et al. (1995) Molecular Cell Biology, Scientific American Books, New York, N.Y., pp. 447-451). For example, Galcheva-Gargova, Z. et al. (1996) Science 272:1797-1802) have identified zinc finger proteins that interact with various cytokine receptors.

The C2H2-type zinc finger signature motif contains a 28 amino acid sequence, including 2 conserved Cys and 2 conserved His residues in a C-2-C-12-H-3-H type motif. The motif generally occurs in multiple tandem repeats. A cysteine-rich domain including the motif Asp-His-His-Cys (DHHC-CRD) has been identified as a distinct subgroup of zinc finger proteins. The DHHC-CRD region has been implicated in growth and development. One DHHC-CRD mutant shows defective function of Ras, a small membrane-associated GTP-binding protein that regulates cell growth and differentiation, while other DHHC-CRD proteins probably function in pathways not involving Ras (Bartels, D. J. et al. (1999) Mol. Cell Biol. 19:6775-6787).

Zinc-finger transcription factors are often accompanied by modular sequence motifs such as the Kruppel-associated box (KRAB) and the SCAN domain. For example, the hypoalphalipoproteinemia susceptibility gene ZNF202 encodes a SCAN box and a KRAB domain followed by eight C2H2 zinc-finger motifs (Honer, C. et al. (2001) Biochim. Biophys. Acta 1517:441-448). The SCAN domain is a highly conserved, leucine-rich motif of approximately 60 amino acids found at the amino-terminal end of zinc finger transcription factors. SCAN domains are most often linked to C2H2 zinc finger motifs through their carboxyl-terminal end. Biochemical binding studies have established the SCAN domain as a selective hetero- and homotypic oligomerization domain. SCAN domain-mediated protein complexes may function to modulate the biological function of transcription factors (Schumacher, C. et al. (2000) J. Biol. Chem. 275:17173-17179).

The KRAB (Kruppel-associated box) domain is a conserved amino acid sequence spanning approximately 75 amino acids and is found in almost one-third of the 300 to 700 genes encoding C2H2 zinc fingers. The KRAB domain is found N-terminally with respect to the finger repeats. The KRAB domain is generally encoded by two exons; the KRAB-A region or box is encoded by one exon and the KRAB-B region or box is encoded by a second exon. The function of the KRAB domain is the repression of transcription. Transcription repression is accomplished by recruitment of either the KRAB-associated protein-I, a transcriptional corepressor, or the KRAB-A interacting protein. Proteins containing the KRAB domain are likely to play a regulatory role during development (Williams, A. J. et al. (1999) Mol. Cell Biol. 19:8526-8535). A subgroup of highly related human KRAB zinc finger proteins detectable in all human tissues is highly expressed in human T lymphoid cells (Bellefroid, E. J. et al. (1993) EMBO J. 12:1363-1374). The ZNF85 KRAB zinc finger gene, a member of the human ZNF91 family, is highly expressed in normal adult testis, in serninomas, and in the NT2/D1 teratocarcinoma cell line (Poncelet, D. A. et al. (1998) DNA Cell Biol. 17:931-943).

The C4 motif is found in hormone-regulated proteins. The C4 motif generally includes only 2 repeats. A number of eukaryotic and viral proteins contain a conserved cysteine-rich domain of 40 to 60 residues (called C3HC4 zinc-finger or RING finger) that binds two atoms of zinc, and is probably involved in mediating protein-protein interactions. The 3D “cross-brace” structure of the zinc ligation system is unique to the RING domain. The spacing of the cysteines in such a domain is C-x(2)-C-x(9 to 39)-C-x(1 to 3)-H-x(2 to3)-C-x(2)-C-x(4 to 48)-C-x(2)-C. The PHD finger is a C4HC3 zinc-finger-like motif found in nuclear proteins thought to be involved in chromatin-mediated transcriptional regulation.

GATA-type transcription factors contain one or two zinc finger domains which bind specifically to a region of DNA that contains the consecutive nucleotide sequence GATA. NMR studies indicate that the zinc finger comprises two irregular anti-parallel β sheets and an α helix, followed by a long loop to the C-terminal end of the finger (Ominchinski, J. G. (1993) Science 261:438-446). The helix and the loop connecting the two β-sheets contact the major groove of the DNA, while the C-terminal part, which determines the specificity of binding, wraps around into the minor groove.

The LIM motif consists of about 60 amino acid residues and contains seven conserved cysteine residues and a histidine within a consensus sequence (Schmeichel, K. L. and Beckerle, M. C. (1994) Cell 79:211-219). The LIM family includes transcription factors and cytoskeletal proteins which may be involved in development, differentiation, and cell growth. One example is actin-binding LIM protein, which may play roles in regulation of the cytoskeleton and cellular morphogenesis (Roof, D. J. et al. (1997) J. Cell Biol. 138:575-588). The N-terminal domain of actin-binding LIM protein has four double zinc finger motifs with the LIM consensus sequence. The C-terminal domain of actin-binding LIM protein shows sequence similarity to known actin-binding proteins such as dematin and villin. Actin-binding LIM protein binds to F-actin through its dematin-like C-terminal domain. The LIM domain may mediate protein-protein interactions with other LIM-binding proteins.

Myeloid cell development is controlled by tissue-specific transcription factors. Myeloid zinc finger proteins (MZF) include MZF-1 and MZF-2. ME-1 functions in regulation of the development of neutrophilic granulocytes. A murine homolog MZF-2 is expressed in myeloid cells, particularly in the cells committed to the neutrophilic lineage. MZF-2 is down-regulated by G-CSF and appears to have a unique function in neutrophil development (Murai, K. et al. (1997) Genes Cells 2:581-591).

The leucine zipper motif comprises a stretch of amino acids rich in leucine which can form an amphipathic α helix. This structure provides the basis for dimerization of two leucine zipper proteins. The region adjacent to the leucine zipper is usually basic, and upon protein dimerization, is optimally positioned for binding to the major groove. Proteins containing such motifs are generally referred to as bZIP transcription factors. The leucine zipper motif is found in the proto-oncogenes Fos and Jun, which comprise the heterodimeric transcription factor API involved in cell growth and the determination of cell lineage (Papavassiliou, A. G. (1995) N. Engl. J. Med. 332:45-47).

The helix-loop-helix motif (HLH) consists of a short α helix connected by a loop to a longer α helix. The loop is flexible and allows the two helices to fold back against each other and to bind to DNA. The transcription factor Myc contains a prototypical HLH motif.

The NF-kappa-B/Rel signature defines a family of eukaryotic transcription factors involved in oncogenesis, embryonic development, differentiation and immune response. Most transcription factors containing the Rel homology domain (RHD) bind as dimers to a consensus DNA sequence motif termed kappa-B. Members of the Rel family share a highly conserved 300 amino acid domain termed the Rel homology domain. The characteristic Rel C-terminal domain is involved in gene activation and cytoplasmic anchoring functions. Proteins known to contain the RHD domain include vertebrate nuclear factor NF-kappa-B, which is a heterodimer of a DNA-binding subunit and the transcription factor p65, mammalian transcription factor RelB, and vertebrate proto-oncogene c-rel, a protein associated with differentiation and lymphopoiesis (Kabrun, N. and Enrietto, P. J. (1994) Serin. Cancer Biol. 5:103-112).

A DNA binding motif termed ARID (AT-rich interactive domain) distinguishes an evolutionarily conserved family of proteins. The approximately 100-residue ARID sequence is present in a series of proteins strongly implicated in the regulation of cell growth, development, and tissue-specific gene expression. ARID proteins include Bright (a regulator of B-cell-specific gene expression), dead ringer (involved in development), and MRF-2 (which represses expression from the cytomegalovirus enhancer) (Dallas, P. B. et al. (2000) Mol. Cell Biol. 20:3137-3146).

The ELM2 (Eg1-27 and MTA1 homology 2) domain is found in metastasis-associated protein MTA1 and protein ER1. The Caenorhabditis elegans gene eg1-27 is required for embryonic patterning MTA1, a human gene with elevated expression in metastatic carcinomas, is a component of a protein complex with histone deacetylase and nucleosome remodelling activities (Solari, F. et al. (1999) Development 126:2483-2494). The ELM2 domain is usually found to the N terminus of a myb-like DNA binding domain. ELM2 is also found associated with an ARID DNA.

The Iroquois (Irx) family of genes are found in nematodes, insects and vertebrates. Irx genes usually occur in one or two genomic clusters of three genes each and encode transcriptional controllers that possess a characteristic homeodomain. The Irx genes function early in development to specify the identity of diverse territories of the body. Later in development in both Drosophila and vertebrates, the Irx genes function again to subdivide those territories into smaller domains. (For a review of Iroquois genes, see Cavodeassi, F. et al. (2001) Development 128:2847-2855.) For example, mouse and human Irx4 proteins are 83% conserved and their 63-aa homeodomain is more than 93% identical to that of the Drosophila Iroquois patterning genes. Irx4 transcripts are predominantly expressed in the cardiac ventricles. The homeobox gene Irx4 mediates ventricular differentiation during cardiac development (Bruneau, B. G. et al. (2000) Dev. Biol. 217:266-77).

Histidine triad (HIT) proteins share residues in distinctive dimeric, 10-stranded half-barrel structures that form two identical purine nucleotide-binding sites. Hint (histidine triad nucleotide-binding protein)-related proteins, found in all forms of life, and fragile histidine triad (Fhit)-related proteins, found in animals and fungi, represent the two main branches of the HIT superfamily. Fhit homologs bind and cleave diadenosine polyphosphates. Fhit-Ap(n)A complexes appear to function in a proapoptotic tumor suppression pathway in epithelial tissues (Brenner C. et al. (1999) J. Cell Physiol.181:179-187).

The peroxisome proliferator-activated receptor gamma (PPAR gamma) is nuclear receptor that controls the expression of a large number of genes involved in adipocyte differentiation, lipid storage and insulin sensitization. PPAR gamma is bound and activated by fatty acid derivatives and prostaglandin J2. Thiazolidinediones are synthetic ligands and agonists of this receptor (Rocchi, S. and Auwerx, J. (2000) Br. J. Nutr. 84:S223-227). Thiazolidinediones or PPAR-gamma agonists improve insulin sensitivity and reduce plasma glucose and blood pressure in subjects with type II diabetes (Lebovitz, H. E. and Banerji, M. A. (2001) Recent Prog. Horm Res. 56:265-294).

Most transcription factors contain characteristic DNA binding motifs, and variations on the above motifs and new motifs have been and are currently being characterized. (Faisst, S. and S. Meyer (1992) Nucleic Acids Res. 20:3-26.)

Chromatin Associated Proteins

Regulation of gene expression depends on bringing the transcriptional machinery to the transcription initiation site of each gene. Proteins which bind DNA can play a role in this regulation. Chromatin proteins can affect the accessibility of regulatory sequences and the initiation site, while transcription factors can increase or decrease the affinity of the RNA polymerase complex for the initiation site. The nuclear DNA of eukaryotes is organized into chromatin, the compact organization of which serves to physically organize DNA as well as to limit the accessibility of DNA to transcription factors, playing a key role in gene regulation (Lewin, supra, pp. 409-410). Two types of chromatin are observed: euchromatin, some of which may be transcribed, and heterochromatin so densely packed that much of it is inaccessible to transcription. The compact structure of chromatin is determined and influenced by chromatin-associated proteins such as the histones, the high mobility group (HMG) proteins, and the chromodomain proteins. There are five classes of histones, H1, H2A, H2B, H3, and H4, all of which are highly basic, low molecular weight proteins. The fundamental unit of chromatin, the nucleosome, consists of 200 base pairs of DNA associated with two copies each of H2A, H2B, H3, and H4. H1 links adjacent nucleosomes. HMG proteins are low molecular weight, non-histone proteins that may play a role in unwinding DNA and stabilizing single-stranded DNA. Chromodomain proteins play a key role in the formation of highly compacted heterochromatin, which is transcriptionally silent. The formation of heterochromatin-like protein complexes also plays a role in the regulation of gene expression and in genome organization. Gene regulation may also occur via modifications such as histone acetylation and DNA methylation which affect chromatin structure.

Chromodomain proteins may be divided into several classes including the Polycomb (Pc) group, the heterochromatin protein 1 (HP1) group, the chromodomain, helicase/ATPase and DNA binding (CHD) group, the SUV39 group, and the retinoblastoma binding protein 1 (RBP1) group. Pc chromodomain proteins are over 300 amino acids long and share a C-terminal region called the Pc-box. Pc proteins function, for example, to suppress Hox genes, the activity of which is limited to a precisely restricted pattern during normal development. Proteins that interact with Pc chromodomain proteins include Ring1A, Bmi-1, Rae-28/Mph1, Me118, and RYBP. HP1-like chromodomain proteins are generally less than 200 amino acids long and share a stretch of negatively charged amino acids near their N-terminus separated by a “hinge” region from a C-terminal region that is a repeat of the chromodomain. Deletion of mammalian Pc1 results in severe proliferative defects in lymphoid cells. Mammalian HP1 proteins include HP1α, HP1β, and HP1γ. Proteins that interact with HP-1 chromodomain proteins include INCENP, TIF1α, BRG1I/SNF2β, H1/H5-like proteins, hRAD54L, bLAP, KAP-1/Tif1β, the Laminin B receptor (LBR), SP100, and CAF-1. Proteins that interact with CHD chromodomnain proteins include HDAC1/2, RbAp46/48, and mta1. Mammalian CHD proteins are about 200 kDa and highly modular, with several sequence motifs that show a consistent position along the length of the proteins. Some members contain PHD Zn fingers. RBP1 contains an ADR domain and a chromodomain. (For a review of chromodomain proteins, see Jones D. O. et al. (2000) BioEssays 22:124-137).

Diseases and Disorders Related to Gene Regulation

Many neoplastic disorders in humans can be attributed to inappropriate gene expression. Malignant cell growth may result from either excessive expression of tumor promoting genes or insufficient expression of tumor suppressor genes. (Cleary, M. L. (1992) Cancer Surv. 15:89-104.) The zinc finger-type transcriptional regulator WT1 is a tumor-suppressor protein that is inactivated in children with Wilm's tumor. The oncogene bc1-6, which plays an important role in large-cell lymphoma, is also a zinc-finger protein (Papavassiliou, A. G. (1995) N. EngI. J. Med. 332:45-47). Chromosomal translocations may also produce chimeric loci that fuse the coding sequence of one gene with the regulatory regions of a second unrelated gene. Such an arrangement likely results in inappropriate gene transcription, potentially contributing to malignancy. In Burkitt's lymphoma, for example, the transcription factor Myc is translocated to the immunoglobulin heavy chain locus, greatly enhancing Myc expression and resulting in rapid cell growth leading to leukemia (Latchman, D. S. (1996) N. Engl. J. Med. 334:28-33).

In addition, the immune system responds to infection or trauma by activating a cascade of events that coordinate the progressive selection, amplification, and mobilization of cellular defense mechanisms. A complex and balanced program of gene activation and repression is involved in this process. However, hyperactivity of the immune system as a result of improper or insufficient regulation of gene expression may result in considerable tissue or organ damage. This damage is well-documented in immunological responses associated with arthritis, allergens, heart attack, stroke, and infections. (Isselbacher et al. Harrison's Principles of Internal Medicine, 13/e, McGraw Hill, Inc. and Teton Data Systems Software, 1996.) The causative gene for autoimmune polyendocrinopathy-candidiasis-ectodermal dystrophy (APECED) was recently isolated and found to encode a protein with two PHD-type zinc finger motifs (Bjorses, P. et al. (1998) Hum. Mol. Genet. 7:1547-1553).

Furthermore, the generation of multicellular organisms is based upon the induction and coordination of cell differentiation at the appropriate stages of development. Central to this process is differential gene expression, which confers the distinct identities of cells and tissues throughout the body. Failure to regulate gene expression during development could result in developmental disorders. Human developmental disorders caused by mutations in zinc finger-type transcriptional regulators include: urogenital developmental abnormalities associated with WT1; Greig cephalopolysyndactyly, Pallister-Hall syndrome, and postaxial polydactyly type A (GLI3), and Townes-Brocks syndrome, characterized by anal, renal, limb, and ear abnormalities (SALL1) (Engelkamp, D. and V. van Heyningen (1996) Curr. Opin. Genet. Dev. 6:334-342; Kohlhase, J. et al. (1999) Am. J. Hum. Genet. 64:435-445).

Pax genes, also called paired-box genes, are a family of developmental control genes that encode nuclear transcription factors. They are characterized by the presence of the paired domain, a conserved amino acid motif with DNA-binding activity. In vertebrates, Pax genes are also involved in embryogenesis. Mutations in four out of nine characterized Pax genes have been associated with congenital human diseases such as Waardenburg syndrome (PAX3), Aniridia (PAX6), Peter's anomaly (PAX6), and renal coloboma syndrome (PAX2). Vertebrate pax genes regulate organogenesis of kidney, eye, ear, nose, limb muscles, vertebral column and brain. Vertebrate Pax genes are involved in pattern formation during embryogenesis (Dahl, E. et al. (1997) Bioessays 19:755-765).

Human acute leukemias involve reciprocal chromosome translocations that fuse the ALL-1 gene located at chromosome region 11q23 to a series of partner genes positioned on a variety of human chromosomes. The fused genes encode chimeric proteins. The AF17 gene encodes a protein of 1093 amino acids, containing a leucine-zipper dimerization motif located 3′ of the fusion point and a cysteine-rich domain at the N terminus that shows homology to a domain within the protein Br140 (peregrin) (Prasad R. et al. (1994) Proc. Natl. Acad. Sci. USA 91:8107-8111). Translin is a DNA binding protein which specifically binds to consensus sequences at breakpoint junctions of chromosomal translocations in many cases of lymphoid malignancies (Aoki, K. et al. (1997) FEBS Lett. 401:109-112).

Synthesis of Nucleic Acids

Polymerases

DNA and RNA replication are critical processes for cell replication and function. DNA and RNA replication are mediated by the enzymes DNA and RNA polymerase, respectively, by a “templating” process in which the nucleotide sequence of a DNA or RNA strand is copied by complementary base-pairing into a complementary nucleic acid sequence of either DNA or RNA. However, there are fundamental differences between the two processes.

DNA polymerase catalyzes the stepwise addition of a deoxyribonucleotide to the 3′-OH end of a polynucleotide strand (the primer strand) that is paired to a second (template) strand. The new DNA strand therefore grows in the 5′ to 3′ direction (Alberts, B. et al. (1994) The Molecular Biology of the Cell, Garland Publishing Inc., New York, N.Y., pp 251-254). The substrates for the polymerization reaction are the corresponding deoxynucleotide triphosphates which must base-pair with the correct nucleotide on the template strand in order to be recognized by the polymerase. Because DNA exists as a double-stranded helix, each of the two strands may serve as a template for the formation of a new complementary strand. Each of the two daughter cells of a dividing cell therefore inherits a new DNA double helix containing one old and one new strand. Thus, DNA is said to be replicated “semiconservatively” by DNA polymerase. In addition to the synthesis of new DNA, DNA polymerase is also involved in the repair of damaged DNA as discussed below under “Ligases.”

In contrast to DNA polymerase, RNA polymerase uses a DNA template strand to “transcribe” DNA into RNA using ribonucleotide triphosphates as substrates. Like DNA polymerization, RNA polymerization proceeds in a 5′ to 3′ direction by addition of a ribonucleoside monophosphate to the 3′-OH end of a growing RNA chain. DNA transcription generates messenger RNAs (mRNA) that carry information for protein synthesis, as well as the transfer, ribosomal, and other RNAs that have structural or catalytic functions. In eukaryotes, three discrete RNA polymerases synthesize the three different types of RNA (Alberts, supra, pp. 367-368). RNA polymerase I makes the large ribosomal RNAs, RNA polymerase II makes the mRNAs that will be translated into proteins, and RNA polymerase III makes a variety of small, stable RNAs, including 5S ribosomal RNA and the transfer RNAs (tRNA). In all cases, RNA synthesis is initiated by binding of the RNA polymerase to a promoter region on the DNA and synthesis begins at a start site within the promoter. Synthesis is completed at a stop (termination) signal in the DNA whereupon both the polymerase and the completed RNA chain are released.

Ligases

DNA repair is the process by which accidental base changes, such as those produced by oxidative damage, hydrolytic attack, or uncontrolled methylation of DNA, are corrected before replication or transcription of the DNA can occur. Because of the efficiency of the DNA repair process, fewer than one in a thousand accidental base changes causes a mutation (Alberts, supra, pp. 245-249). The three steps common to most types of DNA repair are (1) excision of the damaged or altered base or nucleotide by DNA nucleases, (2) insertion of the correct nucleotide in the gap left by the excised nucleotide by DNA polymerase using the complementary strand as the template and, (3) sealing the break left between the inserted nucleotide(s) and the existing DNA strand by DNA ligase. In the last reaction, DNA ligase uses the energy from ATP hydrolysis to activate the 5′ end of the broken phosphodiester bond before forming the new bond with the 3′-OH of the DNA strand. In Bloom's syndrome, an inherited human disease, individuals are partially deficient in DNA ligation and consequently have an increased incidence of cancer (Alberts, supra p. 247).

Nucleases

Nucleases comprise enzymes that hydrolyze both DNA (DNase) and RNA (Rnase). They serve different purposes in nucleic acid metabolism. Nucleases hydrolyze the phosphodiester bonds between adjacent nucleotides either at internal positions (endonucleases) or at the terminal 3′ or 5′ nucleotide positions (exonucleases). A DNA exonuclease activity in DNA polymerase, for example, serves to remove improperly paired nucleotides attached to the 3′-OH end of the growing DNA strand by the polymerase and thereby serves a “proofreading” function. As mentioned above, DNA endonuclease activity is involved in the excision step of the DNA repair process.

RNases also serve a variety of functions. For example, RNase P is a ribonucleoprotein enzyme which cleaves the 5′ end of pre-tRNAs as part of their maturation process. RNase H digests the RNA strand of an RNA/DNA hybrid. Such hybrids occur in cells invaded by retroviruses, and RNase H is an important enzyme in the retroviral replication cycle. Pancreatic RNase secreted by the pancreas into the intestine hydrolyzes RNA present in ingested foods. RNase activity in serum and cell extracts is elevated in a variety of cancers and infectious diseases (Schein, C. H. (1997) Nat. Biotechnol. 15:529-536). Regulation of RNase activity is being investigated as a means to control tumor angiogenesis, allergic reactions, viral infection and replication, and fungal infections.

Modifications of Nucleic Acids

Methylases

Methylation of specific nucleotides occurs in both DNA and RNA, and serves different functions in the two macromolecules. Methylation of cytosine residues to form 5-methyl cytosine in DNA occurs specifically in CG sequences which are base-paired with one another in the DNA double-helix. The pattern of methylation is passed from generation to generation during DNA replication by an enzyme called “maintenance methylase” that acts preferentially on those CG sequences that are base-paired with a CG sequence that is already methylated. Such methylation appears to distinguish active from inactive genes by preventing the binding of regulatory proteins that “turn on” the gene, but permiting the binding of proteins that inactivate the gene (Alberts, supra pp. 448-451). In RNA metabolism, “tRNA methylase” produces one of several nucleotide modifications in tRNA that affect the conformation and base-pairing of the molecule and facilitate the recognition of the appropriate mRNA codons by specific tRNAs. The primary methylation pattern is the dimethylation of guanine residues to form N,N-dimethyl guanine.

Helicases and Single-Stranded Binding Proteins

Helicases are enzymes that destabilize and unwind double helix structures in both DNA and RNA. Since DNA replication occurs more or less simultaneously on both strands, the two strands must first separate to generate a replication “fork” for DNA polymerase to act on. Two types of replication proteins contribute to this process, DNA helicases and single-stranded binding proteins. DNA helicases hydrolyze ATP and use the energy of hydrolysis to separate the DNA strands. Single-stranded binding proteins (SSBs) then bind to the exposed DNA strands, without covering the bases, thereby temporarily stabilizing them for templating by the DNA polymerase (Alberts, supra, pp. 255-256).

RNA helicases also alter and regulate RNA conformation and secondary structure. Like the DNA helicases, RNA helicases utilize energy derived from ATP hydrolysis to destabilize and unwind RNA duplexes. The most well-characterized and ubiquitous family of RNA helicases is the DEAD-box family, so named for the conserved B-type ATP-binding motif which is diagnostic of proteins in this family. Over 40 DEAD-box helicases have been identified in organisms as diverse as bacteria, insects, yeast, amphibians, mammals, and plants. DEAD-box helicases function in diverse processes such as translation initiation, splicing, ribosome assembly, and RNA editing, transport, and stability. Examples of these RNA helicases include yeast Drs1 protein, which is involved in ribosomal RNA processing; yeast TIF1 and TIF2 and mammalian eIF-4A, which are essential to the initiation of RNA translation; and human p68 antigen, which regulates cell growth and division (Ripmaster, T. L. et al. (1992) Proc. Natl. Acad. Sci. USA 89:11131-11135; Chang, T.-H. et al. (1990) Proc. Natl. Acad. Sci. USA 87:1571-1575). These RNA helicases demonstrate strong sequence homology over a stretch of some 420 amino acids. Included among these conserved sequences are the consensus sequence for the A motif of an ATP binding protein; the “DEAD box” sequence, associated with ATPase activity; the sequence SAT, associated with the actual helicase unwinding region; and an octapeptide consensus sequence, required for RNA binding and ATP hydrolysis (Pause, A. et al. (1993) Mol. Cell Biol. 13:6789-6798). Differences outside of these conserved regions are believed to reflect differences in the functional roles of individual proteins (Chang, T. H. et al. (1990) Proc. Natl. Acad. Sci. USA 87:1571-1575).

Some DEAD-box helicases play tissue- and stage-specific roles in spermatogenesis and embryogenesis. Overexpression of the DEAD-box 1 protein (DDX1) may play a role in the progression of neuroblastoma (Nb) and retinoblastoma (Rb) tumors (Godbout, R. et al. (1998) J. Biol. Chem. 273:21161-21168). These observations suggest that DDX1 may promote or enhance tumor progression by altering the normal secondary structure and expression levels of RNA in cancer cells. Other DEAD-box helicases have been implicated either directly or indirectly in tumorigenesis. (Discussed in Godbout, supra.) For example, murine p68 is mutated in ultraviolet light-induced tumors, and human DDX6 is located at a chromosomal breakpoint associated with B-cell lymphoma. Similarly, a chimeric protein comprised of DDX10 and NUP98, a nucleoporin protein, may be involved in the pathogenesis of certain myeloid malignancies.

The RuvA, RuvB, and RuvC proteins play roles in the late stages of homologous genetic recombination and the recombinational repair of damaged DNA. RuvA and RuvB, form a complex that promotes ATP-dependent branch migration of Holliday junctions for the formation of heteroduplex DNA. RuvA acts as a specificity factor that targets RuvB, the branch migration motor to the junction. Two RuvA tetramers sandwich the junction and hold it in an unfolded square-planar configuration. Hexameric rings of RuvB face each other across the junction and promote a novel dual helicase action that “pumps” DNA through the RuvAB complex, using the free energy provided by ATP hydrolysis. The third protein, RuvC endonuclease, resolves the Holliday junction by introducing nicks into two DNA strands. Genetic and biochemical studies indicate that branch migration and resolution are coupled by direct interactions between the three proteins, possibly by the formation of a RuvABC complex (West, S. C.(1997) Annu. Rev. Genet. 31:213-244).

Topoisomerases

Besides the need to separate DNA strands prior to replication, the two strands must be “unwound” from one another prior to their separation by DNA helicases. This function is performed by proteins known as DNA topoisomerases. DNA topoisomerase effectively acts as a reversible nuclease that hydrolyzes a phosphodiesterase bond in a DNA strand, permits the two strands to rotate freely about one another to remove the strain of the helix, and then rejoins the original phosphodiester bond between the two strands. Topoisomerases are essential enzymes responsible for the topological rearrangement of DNA brought about by transcription, replication, chromatin formation, recombination, and chromosome segregation. Superhelical coils are introduced into DNA by the passage of processive enzymes such as RNA polymerase, or by the separation of DNA strands by a helicase prior to replication. Knotting and concatenation can occur in the process of DNA synthesis, storage, and repair. All topoisomerases work by breaking a phosphodiester bond in the ribose-phosphate backbone of DNA. A catalytic tyrosine residue on the enzyme makes a nucleophilic attack on the scissile phosphodiester bond, resulting in a reaction intermediate in which a covalent bond is formed between the enzyme and one end of the broken strand. A tyrosine-DNA phosphodiesterase functions in DNA repair by hydrolyzing this bond in occasional dead-end topoisomerase I-DNA intermediates (Pouliot, J. J. et al. (1999) Science 286:552-555).

Two types of DNA topoisomerase exist, types I and II. Type I topoisomerases work as monomers, making a break in a single strand of DNA while type II topoisomerases, working as homodimers, cleave both strands. DNA Topoisomerase I causes a single-strand break in a DNA helix to allow the rotation of the two strands of the helix about the remaining phosphodiester bond in the opposite strand. DNA topoisomerase II causes a transient break in both strands of a DNA helix where two double helices cross over one another. This type of topoisomerase can efficiently separate two interlocked DNA circles (Alberts, supra, pp.260-262). Type II topoisomerases are largely confined to proliferating cells in eukaryotes, such as cancer cells. For this reason they are targets for anticancer drugs. Topoisomerase II has been implicated in multi-drug resistance (MDR) as it appears to aid in the repair of DNA damage inflicted by DNA binding agents such as doxorubicin and vincristine.

The topoisomerase I family includes topoisomerases I and III (topo I and topo III). The crystal structure of human topoisomerase I suggests that rotation about the intact DNA strand is partially controlled by the enzyme. In this “controlled rotation” model, protein-DNA interactions limit the rotation, which is driven by torsional strain in the DNA (Stewart, L. et al. (1998) Science 379:1534-1541). Structurally, topo I can be recognized by its catalytic tyrosine residue and a number of other conserved residues in the active site region. Topo I is thought to function during transcription. Two topo ms are known in humans, and they are homologous to prokaryotic topoisomerase I, with a conserved tyrosine and active site signature specific to this family. Topo III has been suggested to play a role in meiotic recombination. A mouse topo III is highly expressed in testis tissue and its expression increases with the increase in the number of cells in pachytene (Seki, T. et al. (1998) J. Biol. Chem 273:28553-28556).

The topoisomerase II family includes two isozymes (IIα and IIβ) encoded by different genes. Topo II cleaves double stranded DNA in a reproducible, nonrandom fashion, preferentially in an AT rich region, but the basis of cleavage site selectivity is not known. Structurally, topo II is made up of four domains, the first two of which are structurally similar and probably distantly homologous to similar domains in eukaryotic topo I. The second domain bears the catalytic tyrosine, as well as a highly conserved pentapeptide. The IIa isoform appears to be responsible for unlinking DNA during chromosome segregation. Cell lines expressing IIα but not IIβ suggest that IIβ is dispensable in cellular processes; however, IIβ knockout mice died perinatally due to a failure in neural development. That the major abnormalities occurred in predominantly late developmental events (neurogenesis) suggests that IIβ is needed not at mitosis, but rather during DNA repair (Yang, X. et al. (2000) Science 287:131-134).

Topoisomerases have been implicated in a number of disease states, and topoisomerase poisons have proven to be effective anti-tumor drugs for some human malignancies. Topo I is mislocalized in Fanconi's anemia, and may be involved in the chromosomal breakage seen in this disorder (Wunder, E. (1984) Hum. Genet. 68:276-281). Overexpression of a truncated topo III in ataxia-telangiectasia (A-T) cells partially suppresses the A-T phenotype, probably through a dominant negative mechanism. This suggests that topo III is deregulated in A-T (Fritz, E. et al. (1997) Proc. Natl. Acad. Sci. USA 94:4538-4542). Topo III also interacts with the Bloom's Syndrome gene product, and has been suggested to have a role as a tumor suppressor (Wu, L. et al. (2000) J. Biol. Chem. 275:9636-9644). Aberrant topo II activity is often associated with cancer or increased cancer risk. Greatly lowered topo II activity has been found in some, but not all A-T cell lines (Mohamed, R. et al. (1987) Biochem. Biophys. Res. Commun. 149:233-238). On the other hand, topo II can break DNA in the region of the A-T gene (ATM), which controls all DNA damage-responsive cell cycle checkpoints (Kaufmann, W. K. (1998) Proc. Soc. Exp. Biol. Med. 217:327-334). The ability of topoisomerases to break DNA has been used as the basis of antitumor drugs. Topoisomerase poisons act by increasing the number of dead-end covalent DNA-enzyme complexes in the cell, ultimately triggering cell death pathways (Fortune, J. M. and N. Osheroff (2000) Prog. Nucleic Acid Res. Mol. Biol. 64:221-253; Guichard, S. M. and M. K. Danks (1999) Curr. Opin. Oncol. 11:482-489). Antibodies against topo I are found in the serum of systemic sclerosis patients, and the levels of the antibody may be used as a marker of pulmonary involvement in the disease (Diot, E. et al. (1999) Chest 116:715-720). Finally, the DNA binding region of human topo I has been used as a DNA delivery vehicle for gene therapy (Chen, T. Y. et al. (2000) Appl. Microbiol. Biotechnol. 53:558-567).

Recombinases

Genetic recombination is the process of rearranging DNA sequences within an organism's genome to provide genetic variation for the organism in response to changes in the environment. DNA recombination allows variation in the particular combination of genes present in an individual's genome, as well as the timing and level of expression of these genes. (See Alberts, supra pp. 263-273.) Two broad classes of genetic recombination are commonly recognized, general recombination and site-specific recombination. General recombination involves genetic exchange between any homologous pair of DNA sequences usually located on two copies of the same chromosome. The process is aided by enzymes, recombinases, that “nick” one strand of a DNA duplex more or less randomly and permit exchange with a complementary strand on another duplex. The process does not normally change the arrangement of genes in a chromosome. In site-specific recombination, the recombinase recognizes specific nucleotide sequences present in one or both of the recombining molecules. Base-pairing is not involved in this form of recombination and therefore it does not require DNA homology between the recombining molecules. Unlike general recombination, this form of recombination can alter the relative positions of nucleotide sequences in chromosomes.

RNA Metabolism

Ribonucleic acid (RNA) is a linear single-stranded polymer of four nucleotides, ATP, CTP, UTP, and GTP. In most organisms, RNA is transcribed as a copy of deoxyribonucleic acid (DNA), the genetic material of the organism. In retroviruses RNA rather than DNA serves as the genetic material. RNA copies of the genetic material encode proteins or serve various structural, catalytic, or regulatory roles in organisms. RNA is classified according to its cellular localization and function. Messenger RNAs (mRNAs) encode polypeptides. Ribosomal RNAs (rRNAs) are assembled, along with ribosomal proteins, into ribosomes, which are cytoplasmic particles that translate mRNA into polypeptides. Transfer RNAs (tRNAs) are cytosolic adaptor molecules that function in mRNA translation by recognizing both an mRNA codon and the amino acid that matches that codon. Heterogeneous nuclear RNAs (hnRNAs) include mRNA precursors and other nuclear RNAs of various sizes. Small nuclear RNAs (snRNAs) are a part of the nuclear spliceosome complex that removes intervening, non-coding sequences (introns) and rejoins exons in pre-mRNAs.

Proteins are associated with RNA during its transcription from DNA, RNA processing, and translation of mRNA into protein. Proteins are also associated with RNA as it is used for structural, catalytic, and regulatory purposes.

RNA Processing

Ribosomal RNAs (rRNAs) are assembled, along with ribosomal proteins, into ribosomes, which are cytoplasmic particles that translate messenger RNA (mRNA) into polypeptides. The eukaryotic ribosome is composed of a 60S (large) subunit and a 40S (small) subunit, which together form the 80S ribosome. In addition to the 18S, 28S, 5S, and 5.8S rRNAs, ribosomes contain from 50 to over 80 different ribosomal proteins, depending on the organism. Ribosomal proteins are classified according to which subunit they belong (i.e., L, if associated with the large 60S large subunit or S if associated with the small 40S subunit). E. coli ribosomes have been the most thoroughly studied and contain 50 proteins, many of which are conserved in all life forms. The structures of nine ribosomal proteins have been solved to less than 3.0D resolution (i.e., S5, S6, S17, L1, L6, L9, L12, L14, L30), revealing common motifs, such as b-a-b protein folds in addition to acidic and basic RNA-binding motifs positioned between b-strands. Most ribosomal proteins are believed to contact rRNA directly (reviewed in Liljas, A. and Garber, M. (1995) Curr. Opin. Struct. Biol. 5:721-727; see also Woodson, S. A. and Leontis, N. B. (1998) Curr. Opin. Struct. Biol. 8:294-300; Ramakrishnan, V. and White, S. W. (1998) Trends Biochem. Sci. 23:208-212).

Ribosomal proteins may undergo post-translational modifications or interact with other ribosome-associated proteins to regulate translation. For example, the highly homologous 40S ribosomal protein S6 kinases (S6K1 and S6K2) play a key role in the regulation of cell growth by controlling the biosynthesis of translational components which make up the protein synthetic apparatus (including the ribosomal proteins). In the case of S6K 1, at least eight phosphorylation sites are believed to mediate kinase activation in a hierarchical fashion (Dufner and Thomas (1999) Exp. Cell. Res. 253:100-109). Some of the ribosomal proteins, including L1, also function as translational repressors by binding to polycistronic mRNAs encoding ribosomal proteins (reviewed in Liljas, supra and Garber, supra).

Recent evidence suggests that a number of ribosomal proteins have secondary functions independent of their involvement in protein biosynthesis. These proteins function as regulators of cell proliferation and, in some instances, as inducers of cell death. For example, the expression of human ribosomal protein L13a has been shown to induce apoptosis by arresting cell growth in the G2/M phase of the cell cycle. Inhibition of expression of L13a induces apoptosis in target cells, which suggests that this protein is necessary, in the appropriate amount, for cell survival. Similar results have been obtained in yeast where inactivation of yeast homologues of L13a, rp22 and rp23, results in severe growth retardation and death. A closely related ribosomal protein, L7, arrests cells in G1 and also induces apoptosis. Thus, it appears that a subset of ribosomal proteins may function as cell cycle checkpoints and compose a new family of cell proliferation regulators.

Mapping of individual ribosomal proteins on the surface of intact ribosomes is accomplished using 3D immunocryoelectronmicroscopy, whereby antibodies raised against specific ribosomal proteins are visualized. Progress has been made toward the mapping of L1, L7, and L12 while the structure of the intact ribosome has been solved to only 20-25D resolution and inconsistencies exist among different crude structures (Frank, J. (1997) Curr. Opin. Struct. Biol. 7:266-272).

Three distinct sites have been identified on the ribosome. The aminoacyl-tRNA acceptor site (A site) receives charged tRNAs (with the exception of the initiator-tRNA). The peptidyl-tRNA site (P site) binds the nascent polypeptide as the amino acid from the A site is added to the elongating chain. Deacylated tRNAs bind in the exit site (E site) prior to their release from the ribosome. The structure of the ribosome is reviewed in Stryer, L. (1995) Biochemistry, W.H. Freeman and Company, New York N.Y., pp. 888-9081; Lodish, H. et al. (1995) Molecular Cell Biology, Scientific American Books, New York N.Y., pp. 119-138; and Lewin, B (1997) Genes VI, Oxford University Press, Inc. New York, N.Y.).

Various proteins are necessary for processing of transcribed RNAs in the nucleus. Pre-mRNA processing steps include capping at the 5′ end with methylguanosine, polyadenylating the 3′ end, and splicing to remove introns. The primary RNA transript from DNA is a faithful copy of the gene containing both exon and intron sequences, and the latter sequences must be cut out of the RNA transcript to produce a mRNA that codes for a protein. This “splicing” of the mRNA sequence takes place in the nucleus with the aid of a large, multicomponent ribonucleoprotein complex known as a spliceosome. The spliceosomal complex is comprised of five small nuclear ribonucleoprotein particles (snRNPs) designated U1, U2, U4, U5, and U6. Each snRNP contains a single species of snRNA and about ten proteins. The RNA components of some snRNPs recognize and base-pair with intron consensus sequences. The protein components mediate spliceosome assembly and the splicing reaction. Autoantibodies to snRNP proteins are found in the blood of patients with systemic lupus erythematosus (Stryer, L. (1995) Biochemistry, W.H. Freeman and Company, New York N.Y., p. 863).

Heterogeneous nuclear ribonucleoproteins (hnRNPs) have been identified that have roles in splicing, exporting of the mature RNAs to the cytoplasm, and mRNA translation (Biamonti, G. et al. (1998) Clin. Exp. Rheumatol. 16:317-326). Some examples of hnRNPs include the yeast proteins Hrp1p, involved in cleavage and polyadenylation at the 3′ end of the RNA; Cbp80p, involved in capping the 5′ end of the RNA; and Np13p, a homolog of mammalian hnRNP A1, involved in export of mRNA from the nucleus (Shen, E. C. et al. (1998) Genes Dev. 12:679-691). HnRNPs have been shown to be important targets of the autoimmune response in rheumatic diseases (Biamonti, supra).

Many snRNP and hnRNP proteins are characterized by an RNA recognition motif (RRM). (Reviewed in Birney, E. et al. (1993) Nucleic Acids Res. 21:5803-5816.) The RRM is about 80 amino acids in length and forms four β-strands and two α-helices arranged in an α/β sandwich. The RRM contains a core RNP-1 octapeptide motif along with surrounding conserved sequences. In addition to snRNP proteins, examples of RNA-binding proteins which contain the above motifs include heteronuclear ribonucleoproteins which stabilize nascent RNA and factors which regulate alternative splicing. Alternative splicing factors include developmentally regulated proteins, specific examples of which have been identified in lower eukaryotes such as Drosophila melanogaster and Caenorhabditis elegans. These proteins play key roles in developmental processes such as pattern formation and sex determination, respectively. (See, for example, Hodgkin, J. et al. (1994) Development 120:3681-3689.)

The 3′ ends of most eukaryote mRNAs are also posttranscriptionally modified by polyadenylation. Polyadenylation proceeds through two enzymatically distinct steps: (i) the endonucleolytic cleavage of nascent mRNAs at cis-acting polyadenylation signals in the 3′-untranslated (non-coding) region and (ii) the addition of a poly(A) tract to the 5′ mRNA fragment. The presence of cis-acting RNA sequences is necessary for both steps. These sequences include 5′-AAUAAA-3′ located 10-30 nucleotides upstream of the cleavage site and a less well-conserved GU- or U-rich sequence element located 10-30 nucleotides downstream of the cleavage site. Cleavage stimulation factor (CstF), cleavage factor I (CF I), and cleavage factor II (CF II) are involved in the cleavage reaction while cleavage and polyadenylation specificity factor (CPSF) and poly(A) polymerase (PAP) are necessary for both cleavage and polyadenylation. An additional enzyme, poly(A)-binding protein II (PAB II), promotes poly(A) tract elongation (Rüegsegger, U. et al. (1996) J. Biol. Chem. 271:6107-6113; and references within).

Translation

Correct translation of the genetic code depends upon each amino acid forming a linkage with the appropriate transfer RNA (tRNA). The aminoacyl-tRNA synthetases (aaRSs) are essential proteins found in all living organisms. The aaRSs are responsible for the activation and correct attachment of an amino acid with its cognate tRNA, as the first step in protein biosynthesis. Prokaryotic organisms have at least twenty different types of aaRSs, one for each different amino acid, while eukaryotes usually have two aaRSs, a cytosolic form and a mitochondrial form, for each different amino acid. The 20 aaRS enzymes can be divided into two structural classes. Class I enzymes add amino acids to the 2′ hydroxyl at the 3′ end of tRNAs while Class II enzymes add amino acids to the 3′ hydroxyl at the 3′ end of tRNAs. Each class is characterized by a distinctive topology of the catalytic domain. Class I enzymes contain a catalytic domain based on the nucleotide-binding Rossman ‘fold’. In particular, a consensus tetrapeptide motif is highly conserved (Prosite Document PDOC00161, Aminoacyl-transfer RNA synthetases class-I signature). Class I enzymes are specific for arginine, cysteine, glutamic acid, glutamine, isoleucine, leucine, methionine, tyrosine, tryptophan, and valine. Class II enzymes contain a central catalytic domain, which consists of a seven-stranded antiparallel β-sheet domain, as well as N— and C-terminal regulatory domains. Class II enzymes are separated into two groups based on the heterodimeric or homodimeric structure of the enzyme; the latter group is further subdivided by the structure of the N— and C-terminal regulatory domains (Hartlein, M. and Cusack, S. (1995) J. Mol. Evol. 40:519-530). Class II enzymes are specific for alanine, asparagine, aspartic acid, glycine, histidine, lysine, phenylalanine, proline, serine, and threonine.

Certain aaRSs also have editing functions. IleRS, for example, can misactivate valine to form Val-tRNA^{Ile}, but this product is cleared by a hydrolytic activity that destroys the mischarged product. This editing activity is located within a second catalytic site found in the connective polypeptide 1 region (CP1), a long insertion sequence within the Rossman fold domain of Class I enzymes (Schimmel, P. et al. (1998) FASEB J. 12:1599-1609). AaRSs also play a role in tRNA processing. It has been shown that mature tRNAs are charged with their respective amino acids in the nucleus before export to the cytoplasm, and charging may serve as a quality control mechanism to insure the tRNAs are functional (Martinis, S. A. et al. (1999) EMBO J. 18:4591-4596).

Under optimal conditions, polypeptide synthesis proceeds at a rate of approximately 40 amino acid residues per second. The rate of misincorporation during translation in on the order of 10⁻⁴ and is primarily the result of aminoacyl-t-RNAs being charged with the incorrect amino acid. Incorrectly charged tRNA are toxic to cells as they result in the incorporation of incorrect amino acid residues into an elongating polypeptide. The rate of translation is presumed to be a compromise between the optimal rate of elongation and the need for translational fidelity. Mathematical calculations predict that 10⁻⁴ is indeed the maximum acceptable error rate for protein synthesis in a biological system (reviewed in Stryer, supra; and Watson, J. et al. (1987) The Benjamin/Cummings Publishing Co., Inc. Menlo Park, Calif.). A particularly error prone aminoacyl-tRNA charging event is the charging of tRNA^{Gln} with Gln. A mechanism exits for the correction of this mischarging event which likely has its origins in evolution. Gln was among the last of the 20 naturally occurring amino acids used in polypeptide synthesis to appear in nature. Gram positive eubacteria, cyanobacteria, Archeae, and eukaryotic organelles possess a noncanonical pathway for the synthesis of Gln-tRNA^{Gln} based on the transformation of Glu-tRNA^{Gln} (synthesized by Glu-tRNA synthetase, GluRS) using the enzyme Glu-tRNA^{Gln} amidotransferase (Glu-AdT). The reactions involved in the transamidation pathway are as follows (Curnow, A. W. et al. (1997) Nucleic Acids Symposium 36:2-4):

GluRS

tRNA^{Gln}+Glu+ATP→Glu-tRNA^{Gln}+AMP+PPᵢ

Glu-AdT

Glu-tRNA^{Gln}+Gln+ATP→Gln-tRNA^{Gln}+Glu+ADP+P

A similar enzyme, Asp-tRNA^{Asn} amidotransferase, exists in Archaea, which transforms Asp-tRNA^{Asn} to Asn-tRNA^{Asn}. Formylase, the enzyme that transforms Met-tRNA^{fMet} to fMet-tRNA^{fMet} in eubacteria, is likely to be a related enzyme. A hydrolytic activity has also been identified that destroys mischarged Val-tRNA^{Ile} (Schimmel, P. et al. (1998) FASEB J. 12:1599-1609). One likely scenario for the evolution of Glu-AdT in primitive life forms is the absence of a specific glutaminyl-tRNA synthetase (GlnRS), requiring an alternative pathway for the synthesis of Gln-tRNA^{Gln}. In fact, deletion of the Glu-AdT operon in Gram positive bacteria is lethal (Curnow, A. W. et al. (1997) Proc. Natl. Acad. Sci. USA 94:11819-11826). The existence of GluRS activity in other organisms has been inferred by the high degree of conservation in translation machinery in nature; however, GluRS has not been identified in all organisms, including Homo sapiens. Such an enzyme would be responsible for ensuring translational fidelity and reducing the synthesis of defective polypeptides.

In addition to their function in protein synthesis, specific aminoacyl tRNA synthetases also play roles in cellular fidelity, RNA splicing, RNA trafficking, apoptosis, and transcriptional and translational regulation. For example, human tyrosyl-tRNA synthetase can be proteolytically cleaved into two fragments with distinct cytokine activities. The carboxy-terminal domain exhibits monocyte and leukocyte chemotaxis activity as well as stimulating production of myeloperoxidase, tumor necrosis factor α, and tissue factor. The N-terminal domain binds to the interleukin-8 type A receptor and functions as an interleukin-8-like cytokine. Human tyrosyl-tRNA synthetase is secreted from apoptotic tumor cells and may accelerate apoptosis (Wakasugi, K., and Schimmel, P. (1999) Science 284:147-151). Mitochondrial Neurospora crassa TyrRS and S. cerevisiae LeuRS are essential factors for certain group I intron splicing activities, and human mitochondrial LeuRS can substitute for the yeast LeuRS in a yeast null strain. Certain bacterial aaRSs are involved in regulating their own transcription or translation (Martinis, supra). Several aaRSs are able to synthesize diadenosine oligophosphates, a class of signalling molecules with roles in cell proliferation, differentiation, and apoptosis (Kisselev, L. L. et al. (1998) FEBS Lett. 427:157-163; Vartanian, A. et al. (1999) FEBS Lett. 456:175-180).

Autoantibodies against aminoacyl-tRNAs are generated by patients with autoimmune diseases such as rheumatic arthritis, dermatomyositis and polymyositis, and correlate strongly with complicating interstitial lung disease (ILD) (Freist, W. et al. (1999) Biol. Chem. 380:623-646; Freist, W. et al. (1996) Biol. Chem. Hoppe Seyler 377:343-356). These antibodies appear to be generated in response to viral infection, and coxsackie virus has been used to induce experimental viral myositis in animals.

Comparison of aaRS structures between humans and pathogens has been useful in the design of novel antibiotics (Schimmel, supra). Genetically engineered aaRSs have been utilized to allow site-specific incorporation of unnatural amino acids into proteins in vivo (Liu, D. R. et al. (1997) Proc. Natl. Acad. Sci. USA 94:10092-10097).

tRNA Modifications

The modified ribonucleoside, pseudouridine (ψ), is present ubiquitously in the anticodon regions of transfer RNAs (tRNAs), large and small ribosomal RNAs (rRNAs), and small nuclear RNAs (snRNAs). y is the most common of the modified nucleosides (i.e., other than G, A, U, and C) present in tRNAs. Only a few yeast tRNAs that are not involved in protein synthesis do not contain ψ (Cortese, R. et al. (1974) J. Biol. Chem. 249:1103-1108). The enzyme responsible for the conversion of uridine to ψ. pseudouridine synthase (pseudouridylate synthase), was first isolated from Salmonella typhimurium (Arena, F. et al. (1978) Nucleic Acids Res. 5:4523-4536). The enzyme has since been isolated from a number of mammals, including steer and mice (Green, C. J. et al. (1982) J. Biol. Chem. 257:3045-52; and Chen, J. and Patton, J. R. (1999) RNA 5:409-419). tRNA pseudouridine synthases have been the most extensively studied members of the family. They require a thiol donor (e.g., cysteine) and a monovalent cation (e.g., ammonia or potassium) for optimal activity. Additional cofactors or high energy molecules (e.g., ATP or GTP) are not required (Green, supra). Other eukaryotic pseudouridine synthases have been identified that appear to be specific for rRNA (reviewed in Smith, C. M. and Steitz, J. A. (1997) Cell 89:669-672) and a dual-specificity enzyme has been identified that uses both tRNA and rRNA substrates (Wrzesinski, J. et al. (1995) RNA 1: 437-448). The absence of ψ in the anticodon loop of tRNAs results in reduced growth in both bacteria (Singer, C. E. et al. (1972) Nature New Biol. 238:72-74) and yeast (Lecointe, F. (1998) J. Biol. Chem. 273:1316-1323), although the genetic defect is not lethal.

Another ribonucleoside modification that occurs primarily in eukaryotic cells is the conversion of guanosine to N²,N²-dimethylguanosine (m²₂G) at position 26 or 10 at the base of the D-stem of cytosolic and mitochondrial tRNAs. This posttranscriptional modification is believed to stabilize tRNA structure by preventing the formation of alternative tRNA secondary and tertiary structures. Yeast tRNA^{Asp} is unusual in that it does not contain this modification. The modification does not occur in eubacteria, presumably because the structure of tRNAs in these cells and organelles is sequence constrained and does not require posttranscriptional modification to prevent the formation of alternative structures (Steinberg, S. and Cedergren, R. (1995) RNA 1:886-891, and references within). The enzyme responsible for the conversion of guanosine to m²₂G is a 63 kDa S-adenosylmethionine (SAM)-dependent tRNA N²,N²-dimethyl-guanosine methyltransferase (also referred to as the TRM1 gene product and herein referred to as TRM) (Edqvist, J. (1995) Biochimie 77:54-61). The enzyme localizes to both the nucleus and the mitochondria (Li, J-M. et al. (1989) J. Cell Biol. 109:1411-1419). Based on studies with TRM from Xenopus laevis, there appears to be a requirement for base pairing at positions C11-G24 and G10-C25 immediately preceding the G26 to be modified, with other structural features of the tRNA also being required for the proper presentation of the G26 substrate (Edqvist. J. et al. (1992) Nucleic Acids Res. 20:6575-6581). Studies in yeast suggest that cells carrying a weak ochre tRNA suppressor (sup3-i) are unable to suppress translation termination in the absence of TRM activity, suggesting a role for TRM in modifying the frequency of suppression in eukaryotic cells (Niederberger, C. et al. (1999) FEBS Lett. 464:67-70), in addition to the more general function of ensuring the proper three-dimensional structures for tRNA.

Translation Initiation

Initiation of translation can be divided into three stages. The first stage brings an initiator transfer RNA (Met-tRNA_{f}) together with the 40S ribosomal subunit to form the 43S preinitiation complex. The second stage binds the 43S preinitiation complex to the mRNA, followed by migration of the complex to the correct AUG initiation codon. The third stage brings the 60S ribosomal subunit to the 40S subunit to generate an 80S ribosome at the inititation codon. Regulation of translation primarily involves the first and second stage in the initiation process (V. M. Pain (1996) Eur. J. Biochem. 236:747-771).

Several initiation factors, many of which contain multiple subunits, are involved in bringing an initiator tRNA and the 40S ribosomal subunit together. eIF2, a guanine nucleotide binding protein, recruits the initiator tRNA to the 40S ribosomal subunit. Only when eIF2 is bound to GTP does it associate with the initiator tRNA. eIF2B, a guanine nucleotide exchange protein, is responsible for converting eIF2 from the GDP-bound inactive form to the GTP-bound active form. Two other factors, eIF1A and eIF3 bind and stabilize the 40S subunit by interacting with the 18S ribosomal RNA and specific ribosomal structural proteins. eIF3 is also involved in association of the 40S ribosomal subunit with mRNA. The Met-tRNA_{f}, eIF1A, eIF3, and 40S ribosomal subunit together make up the 43S preinitiation complex (Pain, supra).

Additional factors are required for binding of the 43S preinitiation complex to an mRNA molecule, and the process is regulated at several levels. eIF4F is a complex consisting of three proteins: eIF4E, eIF4A, and eIF4G. eIF4E recognizes and binds to the mRNA 5′-terminal m⁷GTP cap, eIF4A is a bidirectional RNA-dependent helicase, and eIF4G is a scaffolding polypeptide. eIF4G has three binding domains. The N-terminal third of eIF4G interacts with eIF4E, the central third interacts with eIF4A, and the C-terminal third interacts with eIF3 bound to the 43S preinitiation complex. Thus, eIF4G acts as a bridge between the 40S ribosomal subunit and the mRNA (M. W. Hentze (1997) Science 275:500-501).

The ability of eIF4F to initiate binding of the 43S preinitiation complex is regulated by structural features of the mRNA. The mRNA molecule has an untranslated region (UTR) between the 5′ cap and the AUG start codon. In some mRNAs this region forms secondary structures that impede binding of the 43S preinitiation complex. The helicase activity of eIF4A is thought to function in removing this secondary structure to facilitate binding of the 43S preinitiation complex (Pain, supra).

Translation Elongation

Elongation is the process whereby additional amino acids are joined to the initiator methionine to form the complete polypeptide chain. The elongation factors EF1α, EF1βγ, and EF2 are involved in elongating the polypeptide chain following initiation. EF1α is a GTP-binding protein. In EF1α's GTP-bound form, it brings an aminoacyl-tRNA to the ribosome's A site. The amino acid attached to the newly arrived aminoacyl-tRNA forms a peptide bond with the initiatior methionine. The GTP on EF1α is hydrolyzed to GDP, and EF1α-GDP dissociates from the ribosome. EF1βγ binds EF1α-GDP and induces the dissociation of GDP from EF1α, allowing EF1α to bind GTP and a new cycle to begin.

As subsequent aminoacyl-tRNAs are brought to the ribosome, EF-G, another GTP-binding protein, catalyzes the translocation of tRNAs from the A site to the P site and finally to the E site of the ribosome. This allows the ribosome and the mRNA to remain attached during translation.

Translation Termination

The release factor eRF carries out termination of translation. eRF recognizes stop codons in the mRNA, leading to the release of the polypeptide chain from the ribosome.

Expression Profiling

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

The discovery of new nucleic acid-associated proteins, and the polynucleotides encoding them, satisfies a need in the art by providing new compositions which are useful in the diagnosis, prevention, and treatment of cell proliferative, neurological, developmental, and autoimmune/inflammatory disorders, and infections, and in the assessment of the effects of exogenous compounds on the expression of nucleic acid and amino acid sequences of nucleic acid-associated proteins.

### SUMMARY OF THE INVENTION

The invention features purified polypeptides, nucleic acid-associated proteins, referred to collectively as “NAAP” and individually as “NAAP-1,” “NAAP-2,” “NAP-3,” “NAA4,” “NAAP-5,” “NAAP-6,” “NAAP-7,” “NAAP-8,” “NAAP-9,” “NAAP-10,” “NAAP-11,” “NAAP-12,” “NAAP-13,” “NAAP-14,” “NAAP-15,” “NAAP-16,” “NAAP-17,” “NAAP-18,” “NAAP-19,” “NAAP-20,” “NAAP-21,” “NAAP-22,” and “NAAP-23.” In one aspect, the invention provides an isolated polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. In one alternative, the invention provides an isolated polypeptide comprising the amino acid sequence of SEQ ID NO:1-23.

The invention further provides an isolated polynucleotide encoding a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. In one alternative, the polynucleotide encodes a polypeptide selected from the group consisting of SEQ ID NO:1-23. In another alternative, the polynucleotide is selected from the group consisting of SEQ ID NO:24-46.

Additionally, the invention provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. In one alternative, the invention provides a cell transformed with the recombinant polynucleotide. In another alternative, the invention provides a transgenic organism comprising the recombinant polynucleotide.

The invention also provides a method for producing a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23.

The invention further provides an isolated polynucleotide selected from the group consisting of a) a polynucleotide comprising a polynucleotide sequence selected from the group consisting of SEQ ID NO:24-46, b) a polynucleotide comprising a naturally occurring polynucleotide sequence at least 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:24-46, b) a polynucleotide comprising a naturally occurring polynucleotide sequence at least 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide selected from the group consisting of a) a polynucleotide comprising a polynucleotide sequence selected from the group consisting of SEQ ID NO:24-46, b) a polynucleotide comprising a naturally occurring polynucleotide sequence at least 90% identical to a polynucleotide sequence selected from the group consisting of SEQ ID NO:24-46, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23, and a pharmaceutically acceptable excipient. In one embodiment, the composition comprises an amino acid sequence selected from the group consisting of SEQ ID NO:1-23. The invention additionally provides a method of treating a disease or condition associated with decreased expression of functional NAAP, comprising administering to a patient in need of such treatment the composition.

The invention also provides a method for screening a compound for effectiveness as an agonist of a polypeptide selected from the group consisting of a) a polypeptide comprising an amino acid sequence selected from the group consisting of SEQ D) NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional NAAP, comprising administering to a patient in need of such treatment the composition.

Additionally, the invention provides a method for screening a compound for effectiveness as an antagonist of a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional NAAP, comprising administering to a patient in need of such treatment the composition.

The invention further provides a method of screening for a compound that specifically binds to a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide selected from the group consisting of a) a polypeptide comprising an amino acid sequence selected from the group consisting of SEQ ID NO:1-23, b) a polypeptide comprising a naturally occurring amino acid sequence at least 90% identical to an amino acid sequence selected from the group consisting of SEQ ID NO:1-23, c) a biologically active fragment of a polypeptide having an amino acid sequence selected from the group consisting of SEQ ID NO:1-23, and d) an immunogenic fragment of a polypeptide having an amino acid sequence selected from the group consisting of SEQ ID NO:1-23. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a polynucleotide sequence selected from the group consisting of SEQ ID NO:24-46, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:24-46, ii) a polynucleotide comprising a naturally occurring polynucleotide sequence at least 90% identical to a polynucleotide sequence selected from the group consisting of SEQ ID NO:24-46, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:24-46, ii) a polynucleotide comprising a naturally occurring polynucleotide sequence at least 90% identical to a polynucleotide sequence selected from the group consisting of SEQ ID NO:24-46, iii) a polynucleotide complementary to the polynucleotide of i), iv) a polynucleotide complementary to the polynucleotide of ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for the full length polynucleotide and polypeptide sequences of the present invention.

Table 2 shows the GenBank identification number and annotation of the nearest GenBank homolog for polypeptides of the invention. The probability scores for the matches between each polypeptide and its homolog(s) are also shown.

Table 3 shows structural features of polypeptide sequences of the invention, including predicted motifs and domains, along with the methods, algorithms, and searchable databases used for analysis of the polypeptides.

Table 4 lists the cDNA and/or genomic DNA fragments which were used to assemble polynucleotide sequences of the invention, along with selected fragments of the polynucleotide sequences.

Table 5 shows the representative cDNA library for polynucleotides of the invention.

Table 6 provides an appendix which describes the tissues and vectors used for construction of the cDNA libraries shown in Table 5.

Table 7 shows the tools, programs, and algorithms used to analyze the polynucleotides and polypeptides of the invention, along with applicable descriptions, references, and threshold parameters.

### DESCRIPTION OF THE INVENTION

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“NAAP” refers to the amino acid sequences of substantially purified NAAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of NAAP. Agonists may include proteins, nucleic acids, carbohydrates, small molecules, or any other compound or composition which modulates the activity of NAAP either by directly interacting with NAAP or by acting on components of the biological pathway in which NAAP participates.

An “allelic variant” is an alternative form of the gene encoding NAAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding NAAP include those sequences with deletions, insertions, or substitutions of different nucleotides, resulting in a polypeptide the same as NAAP or a polypeptide with at least one functional characteristic of NAAP. Included within this definition are polymorphisms which may or may not be readily detectable using a particular oligonucleotide probe of the polynucleotide encoding NAAP, and improper or unexpected hybridization to allelic variants, with a locus other than the normal chromosomal locus for the polynucleotide sequence encoding NAAP. The encoded protein may also be “altered,” and may contain deletions, insertions, or substitutions of amino acid residues which produce a silent change and result in a functionally equivalent NAAP. Deliberate amino acid substitutions may be made on the basis of similarity in polarity, charge, solubility, hydrophobicity, hydrophilicity, and/or the amphipathic nature of the residues, as long as the biological or immunological activity of NAAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of NAAP. Antagonists may include proteins such as antibodies, nucleic acids, carbohydrates, small molecules, or any other compound or composition which modulates the activity of NAAP either by directly interacting with NAAP or by acting on components of the biological pathway in which NAAP participates.

The term “antibody” refers to intact immunoglobulin molecules as well as to fragments thereof, such as Fab, F(ab′)₂, and Fv fragments, which are capable of binding an epitopic determinant. Antibodies that bind NAAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic NAAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding NAAP or fragments of NAAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original | Conservative |
|---|---|
| Residue | Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of NAAP or the polynucleotide encoding NAAP which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:24-46 comprises a region of unique polynucleotide sequence that specifically identifies SEQ ID NO:24-46, for example, as distinct from any other sequence in the genome from which the fragment was obtained. A fragment of SEQ ID NO:24-46 is useful, for example, in hybridization and amplification technologies and in analogous methods that distinguish SEQ ID NO:24-46 from related polynucleotide sequences. The precise length of a fragment of SEQ ID NO:24-46 and the region of SEQ ID NO:24-46 to which the fragment corresponds are routinely determinable by one of ordinary skill in the art based on the intended purpose for the fragment.

A fragment of SEQ ID NO:1-23 is encoded by a fragment of SEQ ID NO:24-46. A fragment of SEQ ID NO:1-23 comprises a region of unique amino acid sequence that specifically identifies SEQ ID NO:1-23. For example, a fragment of SEQ ID NO:1-23 is useful as an immunogenic peptide for the development of antibodies that specifically recognize SEQ ID NO:1-23. The precise length of a fragment of SEQ ID NO:1-23 and the region of SEQ ID NO:1-23 to which the fragment corresponds are routinely determinable by one of ordinary skill in the art based on the intended purpose for the fragment.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=l, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of NAAP which is capable of eliciting an immune response when introduced into a living organism, for example, a mammal. The term “immunogenic fragment” also includes any polypeptide or oligopeptide fragment of NAAP which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of NAAP. For example, modulation may cause an increase or a decrease in protein activity, binding characteristics, or any other biological, functional, or immunological properties of NAAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an NAAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of NAAP.

“Probe” refers to nucleic acid sequences encoding NAAP, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols. A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose.

The term “sample” is used in its broadest sense. A sample suspected of containing NAAP, nucleic acids encoding NAAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In one alternative, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotide sequences that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

The invention is based on the discovery of new human nucleic acid-associated proteins (NAAP), the polynucleotides encoding NAAP, and the use of these compositions for the diagnosis, treatment, or prevention of cell proliferative, neurological, developmental, and autoimmunelinflammatory disorders, and infections.

Table 1 summarizes the nomenclature for the full length polynucleotide and polypeptide sequences of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are nucleic acid-associated proteins. For example, SEQ ID NO:2 is 42% identical, from residue Q784 to residue F1175, to thale cress putative ATP-dependent RNA helicase A (GenBank ID g4510377) as determined by the Basic Local Alignment Search Tool (BLAST). (See Table 2.) The BLAST probability score is 1.9e-168, which indicates the probability of obtaining the observed polypeptide sequence alignment by chance. SEQ ID NO:2 also contains a DEAD/DEAH box helicase domain as determined by searching for statistically significant matches in the hidden Markov model (M)-based PFAM database of conserved protein family domains. (See Table 3.) Data from BLIMPS, MOTIFS, and PROFILECAN analyses provide further corroborative evidence that SEQ ID NO:2 is a DEAD/DEAH box helicase.

In another example, SEQ ID NO:4 is 37% identical from residue L128 to residue P513, 58% identical from residue F624 to residue L707, and 26% identical from residue K9 to residue Y49 to Mus musculus ERG-associated protein ESET (GenBank ID g3644042) as determined by the Basic Local Alignment Search Tool (BLAST). (See Table 2.) The BLAST probability score is 1.5e-88, which indicates the probability of obtaining the observed polypeptide sequence alignment by chance. SEQ ID NO:4 also contains a SET domain as determined by searching for statistically significant matches in the hidden Markov model (HMM)-based PFAM database of conserved protein family domains. (See Table 3.) Data from BLIMPS, and BLAST analyses of the PRODOM and DOMO databases provide further corroborative evidence that SEQ ID NO:4 contains a SET domain and is a transcription factor.

In another example, SEQ ID NO:8 is 100% identical, from residue M221 to residue C665, to human putative helicase RUVBL (GenBank ID g8886769) as determined by the Basic Local Alignment Search Tool (BLAST). (See Table 2.) The BLAST probability score is 8.0e-239, which indicates the probability of obtaining the observed polypeptide sequence alignment by chance. SEQ ID NO:8 also contains an ATPase domain as determined by searching for statistically significant matches in the hidden Markov model (HMM)-based PFAM database of conserved protein family domains. (See Table 3.) Data from BLIMPS and MOTIFS analyses provide further corroborative evidence that SEQ ID NO:8 is a helicase.

In a further example, SEQ ID NO:14 is 98% identical, from residue F13 to residue K312, to human paired-box (PAX) protein (GenBank ID g409139) as determined by the Basic Local Alignment Search Tool (BLAST). (See Table 2.) The BLAST probability score is 1.4e-156, which indicates the probability of obtaining the observed polypeptide sequence alignment by chance. SEQ ID NO:14 also contains a paired-box domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:14 is a PAX protein. SEQ ID NO:1, SEQ ID NO:3, SEQ ID NO:5-7, SEQ ID NO:9-13 and SEQ ID NO:15-23 were analyzed and annotated in a similar manner. The algorithms and parameters for the analysis of SEQ ID NO:1-23 are described in Table 7.

As shown in Table 4, the full length polynucleotide sequences of the present invention were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide sequences of the invention, and of fragments of the polynucleotide sequences which are useful, for example, in hybridization or amplification technologies that identify SEQ ID NO:24-46 or that distinguish between SEQ ID NO:24-46 and related polynucleotide sequences.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotide sequences. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithmn, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| | Type of analysis and/or examples |
|---|---|
| Prefix | of programs |
| GNN, GFG, ENST | Exon prediction from genomic |
| | sequences using, for example, |
| | GENSCAN (Stanford University, CA, |
| | USA) or FGENES (Computer |
| | Genomics Group, The Sanger Centre, |
| | Cambridge, UK). |
| GBI | Hand-edited analysis of genomic |
| | sequences. |
| FL | Stitched or stretched genomic |
| | sequences (see Example V). |
| INCY | Full length transcript and exon |
| | prediction from mapping of EST |
| | sequences to the genome. Genomic |
| | location and EST composition data |
| | are combined to predict the |
| | exons and resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotide sequences which were assembled using Incyte cDNA sequences. The representative cDNA library is the Incyte cDNA library which is most frequently represented by the Incyte cDNA sequences which were used to assemble and confirm the above polynucleotide sequences. The tissues and vectors which were used to construct the cDNA libraries shown in Table 5 are described in Table 6.

The invention also encompasses NAAP variants. A preferred NAAP variant is one which has at least about 80%, or alternatively at least about 90%, or even at least about 95% amino acid sequence identity to the NAAP amino acid sequence, and which contains at least one functional or structural characteristic of NAAP.

The invention also encompasses polynucleotides which encode NAAP. In a particular embodiment, the invention encompasses a polynucleotide sequence comprising a sequence selected from the group consisting of SEQ ID NO:24-46, which encodes NAAP. The polynucleotide sequences of SEQ ID NO:24-46, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding NAAP. In particular, such a variant polynucleotide sequence will have at least about 70%, or alternatively at least about 85%, or even at least about 95% polynucleotide sequence identity to the polynucleotide sequence encoding NAAP. A particular aspect of the invention encompasses a variant of a polynucleotide sequence comprising a sequence selected from the group consisting of SEQ ID NO:24-46 which has at least about 70%, or alternatively at least about 85%, or even at least about 95% polynucleotide sequence identity to a nucleic acid sequence selected from the group consisting of SEQ ID NO:24-46. Any one of the polynucleotide variants described above can encode an amino acid sequence which contains at least one functional or structural characteristic of NAAP.

In addition, or in the alternative, a polynucleotide variant of the invention is a splice variant of a polynucleotide sequence encoding NAAP. A splice variant may have portions which have significant sequence identity to the polynucleotide sequence encoding NAAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to the polynucleotide sequence encoding NAAP over its entire length; however, portions of the splice variant will have at least about 70%, or alternatively at least about 85%, or alternatively at least about 95%, or alternatively 100% polynucleotide sequence identity to portions of the polynucleotide sequence encoding NAAP. Any one of the splice variants described above can encode an amino acid sequence which contains at least one functional or structural characteristic of NAAP.

It will be appreciated by those skilled in the art that as a result of the degeneracy of the genetic code, a multitude of polynucleotide sequences encoding NAAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring NAAP, and all such variations are to be considered as being specifically disclosed.

Although nucleotide sequences which encode NAAP and its variants are generally capable of hybridizing to the nucleotide sequence of the naturally occurring NAAP under appropriately selected conditions of stringency, it may be advantageous to produce nucleotide sequences encoding NAAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NAAP and its derivatives without altering the encoded amino acid sequences include the production of RNA transcripts having more desirable properties, such as a greater half-life, than transcripts produced from the naturally occurring sequence.

The invention also encompasses production of DNA sequences which encode NAAP and NAAP derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding NAAP or any fragment thereof.

Also encompassed by the invention are polynucleotide sequences that are capable of hybridizing to the claimed polynucleotide sequences, and, in particular, to those shown in SEQ ID NO:24-46 and fragments thereof under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding NAAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, eg., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode NAAP may be cloned in recombinant DNA molecules that direct expression of NAAP, or fragments or functional equivalents thereof, in appropriate host cells. Due to the inherent degeneracy of the genetic code, other DNA sequences which encode substantially the same or a functionally equivalent amino acid sequence may be produced and used to express NAAP.

The nucleotide sequences of the present invention can be engineered using methods generally known in the art in order to alter NAAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of NAAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding NAAP may be synthesized, in whole or in part, using chemical methods well known in the art. (See, e.g., Caruthers, M. H. et al. (1980) Nucleic Acids Symp. Ser. 7:215-223; and Hom, T. et al. (1980) Nucleic Acids Symp. Ser. 7:225-232.) Alternatively, NAAP itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of NAAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active NAAP, the nucleotide sequences encoding NAAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding NAAP. Such elements may vary in their strength and specificity. Specific initiation signals may also be used to achieve more efficient translation of sequences encoding NAAP. Such signals include the ATG initiation codon and adjacent sequences, e.g. the Kozak sequence. In cases where sequences encoding NAAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding NAAP and appropriate transcriptional and translational control elements. These methods include in vitro recombinant DNA techniques, synthetic techniques, and in vivo genetic recombination. (See, e.g., Sambrook, J. et al. (1989) Molecular Cloning. A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding NAAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350-356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding NAAP. For example, routine cloning, subcloning, and propagation of polynucleotide sequences encoding NAAP can be achieved using a multifunctional E. coli vector such as PBLUESCRIPT (Stratagene, La Jolla Calif.) or PSPORT1 plasmid (Life Technologies). Ligation of sequences encoding NAAP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of NAAP are needed, e.g. for the production of antibodies, vectors which direct high level expression of NAAP may be used. For example, vectors containing the strong, inducible SP6 or T7 bacteriophage promoter may be used.

Yeast expression systems may be used for production of NAAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of NAAP. Transcription of sequences encoding NAAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding NAAP may be ligated into an adenovirus transcription/translation complex consisting of the late promoter and tripartite leader sequence. Insertion in a non-essential E1 or E3 region of the viral genome may be used to obtain infective virus which expresses NAAP in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of NAAP in cell lines is preferred. For example, sequences encoding NAAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding NAAP is inserted within a marker gene sequence, transformed cells containing sequences encoding NAAP can be identified by the absence of marker gene function. Alternatively, a marker gene can be placed in tandem with a sequence encoding NAAP under the control of a single promoter. Expression of the marker gene in response to induction or selection usually indicates expression of the tandem gene as well.

In general, host cells that contain the nucleic acid sequence encoding NAAP and that express NAAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of NAAP using either specific polyclonal or monoclonal antibodies are known in the art. Examples of such techniques include enzyme-linked immnunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on NAAP is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding NAAP include oligolabeling, nick translation, end-labeling, or PCR amplification using a labeled nucleotide. Alternatively, the sequences encoding NAAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding NAAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode NAAP may be designed to contain signal sequences which direct secretion of NAAP through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding NAAP may be ligated to a heterologous sequence resulting in translation of a fusion protein in any of the aforementioned host systems. For example, a chimeric NAAP protein containing a heterologous moiety that can be recognized by a commercially available antibody may facilitate the screening of peptide libraries for inhibitors of NAAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the NAAP encoding sequence and the heterologous protein sequence, so that NAAP may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled NAAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

NAAP of the present invention or fragments thereof may be used to screen for compounds that specifically bind to NAAP. At least one and up to a plurality of test compounds may be screened for specific binding to NAAP. Examples of test compounds include antibodies, oligonucleotides, proteins (e.g., receptors), or small molecules.

In one embodiment, the compound thus identified is closely related to the natural ligand of NAAP, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which NAAP binds, or to at least a fragment of the receptor, e.g., the ligand binding site. In either case, the compound can be rationally designed using known techniques. In one embodiment, screening for these compounds involves producing appropriate cells which express NAAP, either as a secreted protein or on the cell membrane. Preferred cells include cells from mammals, yeast, Drosophila, or E. coli. Cells expressing NAAP or cell membrane fractions which contain NAAP are then contacted with a test compound and binding, stimulation, or inhibition of activity of either NAAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with NAAP, either in solution or affixed to a solid support, and detecting the binding of NAAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

NAAP of the present invention or fragments thereof may be used to screen for compounds that modulate the activity of NAAP. Such compounds may include agonists, antagonists, or partial or inverse agonists. In one embodiment, an assay is performed under conditions permissive for NAAP activity, wherein NAAP is combined with at least one test compound, and the activity of NAAP in the presence of a test compound is compared with the activity of NAAP in the absence of the test compound. A change in the activity of NAAP in the presence of the test compound is indicative of a compound that modulates the activity of NAAP. Alternatively, a test compound is combined with an in vitro or cell-free system comprising NAAP under conditions suitable for NAAP activity, and the assay is performed. In either of these assays, a test compound which modulates the activity of NAAP may do so indirectly and need not come in direct contact with the test compound. At least one and up to a plurality of test compounds may be screened.

In another embodiment, polynucleotides encoding NAAP or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and mnicroinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding NAAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding NAAP can also be used to create “knockin” humanized animals (pigs) or transgenic animals (mice or rats) to model human disease. With knockin technology, a region of a polynucleotide encoding NAAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress NAAP, e.g., by secreting NAAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of NAAP and nucleic acid-associated proteins. In addition, examples of tissues expressing NAAP can be found in Table 6 and can also be found in Example XL. Therefore, NAAP appears to play a role in cell proliferative, neurological, developmental, and autoimmune/inflammatory disorders, and infections. In the treatment of disorders associated with increased NAAP expression or activity, it is desirable to decrease the expression or activity of NAAP. In the treatment of disorders associated with decreased NAAP expression or activity, it is desirable to increase the expression or activity of NAAP.

Therefore, in one embodiment, NAAP or a fragment or derivative thereof may be administered to a subject to treat or prevent a disorder associated with decreased expression or activity of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helrinthic infections, and trauma; and an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm.

In another embodiment, a vector capable of expressing NAAP or a fragment or derivative thereof may be administered to a subject to treat or prevent a disorder associated with decreased expression or activity of NAAP including, but not limited to, those described above.

In a further embodiment, a composition comprising a substantially purified NAAP in conjunction with a suitable pharmaceutical carrier may be administered to a subject to treat or prevent a disorder associated with decreased expression or activity of NAAP including, but not limited to, those provided above.

In still another embodiment, an agonist which modulates the activity of NAAP may be administered to a subject to treat or prevent a disorder associated with decreased expression or activity of NAAP including, but not limited to, those listed above.

In a further embodiment, an antagonist of NAAP may be administered to a subject to treat or prevent a disorder associated with increased expression or activity of NAAP. Examples of such disorders include, but are not limited to, those cell proliferative, neurological, developmental, and autoimmune/inflammatory disorders, and infections described above. In one aspect, an antibody which specifically binds NAAP may be used directly as an antagonist or indirectly as a targeting or delivery mechanism for bringing a pharmaceutical agent to cells or tissues which express NAAP.

In an additional embodiment, a vector expressing the complement of the polynucleotide encoding NAAP may be administered to a subject to treat or prevent a disorder associated with increased expression or activity of NAAP including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of NAAP may be produced using methods which are generally known in the art. In particular, purified NAAP may be used to produce antibodies or to screen libraries of pharmaceutical agents to identify those which specifically bind NAAP. Antibodies to NAAP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermnans, S. (2001) J. Biotechnol. 74:277-302).

For the production of antibodies, various hosts including goats, rabbits, rats, mice, camels, dromedaries, llamas, humans, and others may be immunized by injection with NAAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NAAP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of NAAP amino acids may be fused with those of another protein, such as KLH, and antibodies to the chimeric molecule may be produced.

Monoclonal antibodies to NAAP may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. NatI. Acad. Sci. USA 81:6851-6855; Neuberger, M. S. et al. (1984) Nature 312:604-608; and Takeda, S. et al. (1985) Nature 314:452454.) Alternatively, techniques described for the production of single chain antibodies may be adapted, using methods known in the art, to produce NAAP-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for NAAP may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NAAP and its specific antibody. A two-site, monoclonal-based immunoassay utilizing monoclonal antibodies reactive to two non-interfering NAAP epitopes is generally used, but a competitive binding assay may also be employed (Pound, supra).

Various methods such as Scatchard analysis in conjunction with radioimmunoassay techniques may be used to assess the affinity of antibodies for NAAP. Affinity is expressed as an association constant, Kₐ, which is defined as the molar concentration of NAAP-antibody complex divided by the molar concentrations of free antigen and free antibody under equilibrium conditions. The Kₐ determined for a preparation of polyclonal antibodies, which are heterogeneous in their affinities for multiple NAAP epitopes, represents the average affinity, or avidity, of the antibodies for NAAP. The Kₐ determined for a preparation of monoclonal antibodies, which are monospecific for a particular NAAP epitope, represents a true measure of affinity. High-affinity antibody preparations with Kₐ ranging from about 10⁹ to 10¹² L/mole are preferred for use in immunoassays in which the NAAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of NAAP, preferably in active form, from the antibody (Catty, D. (1988) Antibodies. Volume I: A Practical Approach, IRL Press, Washington DC; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NAAP-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, the polynucleotides encoding NAAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding NAAP. Such technology is well known in the art, and antisense oligonucleotides or larger fragments can be designed from various locations along the coding or control regions of sequences encoding NAAP. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding NAAP may be used for somatic of germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in NAAP expression or regulation causes disease, the expression of NAAP from an appropriate population of transduced cells may alleviate the clinical manifestations caused by the genetic deficiency.

In a further embodiment of the invention, diseases or disorders caused by deficiencies in NAAP are treated by constructing mammalian expression vectors encoding NAAP and introducing these vectors by mechanical means into NAAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem 62:191-217; Ivics, Z. (1997) Cell 91:501-510; Boulay, J-L. and H. Recipon (1998) Curr. Opin. Biotechnol. 9:445-450).

Expression vectors that may be effective for the expression of NAAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). NAAP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding NAAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to NAAP expression are treated by constructing a retrovirus vector consisting of (i) the polynucleotide encoding NAAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding NAAP to cells which have one or more genetic abnormalities with respect to the expression of NAAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another alternative, a herpes-based, gene therapy delivery system is used to deliver polynucleotides encoding NAAP to target cells which have one or more genetic abnormalities with respect to the expression of NAAP. The use of herpes simplex virus (HSV)-based vectors may be especially valuable for introducing NAAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No.5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding NAAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for NAAP into the alphavirus genome in place of the capsid-coding region results in the production of a large number of NAAP-coding RNAs and the synthesis of high levels of NAAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of NAAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding NAAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding NAAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding NAAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased NAAP expression or activity, a compound which specifically inhibits expression of the polynucleotide encoding NAAP may be therapeutically useful, and in the treatment of disorders associated with decreased NAAP expression or activity, a compound which specifically promotes expression of the polynucleotide encoding NAAP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-ocurring or non-natural chemical compounds; rational design of a compound based on chemical and/or structural properties of the target polynucleotide; and selection from a library of chemical compounds created combinatorially or randomly. A sample comprising a polynucleotide encoding NAAP is exposed to at least one test compound thus obtained. The sample may comprise, for example, an intact or permeabilized cell, or an in vitro cell-free or reconstituted biochemical system. Alterations in the expression of a polynucleotide encoding NAAP are assayed by any method commonly known in the art. Typically, the expression of a specific nucleotide is detected by hybridization with a probe having a nucleotide sequence complementary to the sequence of the polynucleotide encoding NAAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of NAAP, antibodies to NAAP, and mimetics, agonists, antagonists, or inhibitors of NAAP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising NAAP or fragments thereof. For example, liposome preparations containing a cell-impermeable macromolecule may promote cell fusion and intracellular delivery of the macromolecule. Alternatively, NAAP or a fragment thereof may be joined to a short cationic N-terminal portion from the HV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example NAAP or fragments thereof, antibodies of NAAP, and agonists, antagonists or inhibitors of NAAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind NAAP may be used for the diagnosis of disorders characterized by expression of NAAP, or in assays to monitor patients being treated with NAAP or agonists, antagonists, or inhibitors of NAAP. Antibodies useful for diagnostic purposes may be prepared in the same manner as described above for therapeutics. Diagnostic assays for NAAP include methods which utilize the antibody and a label to detect NAAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring NAAP, including ELISAs, RIAs, and FACS, are known in the art and provide a basis for diagnosing altered or abnormal levels of NAAP expression. Normal or standard values for NAAP expression are established by combining body fluids or cell extracts taken from normal mammalian subjects, for example, human subjects, with antibodies to NAAP under conditions suitable for complex formation. The amount of standard complex formation may be quantitated by various methods, such as photometric means. Quantities of NAAP expressed in subject, control, and disease samples from biopsied tissues are compared with the standard values. Deviation between standard and subject values establishes the parameters for diagnosing disease.

In another embodiment of the invention, the polynucleotides encoding NAAP may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of NAAP may be correlated with disease. The diagnostic assay may be used to determine absence, presence, and excess expression of NAAP, and to monitor regulation of NAAP levels during therapeutic intervention.

In one aspect, hybridization with PCR probes which are capable of detecting polynucleotide sequences, including genomic sequences, encoding NAAP or closely related molecules may be used to identify nucleic acid sequences which encode NAAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding NAAP, allelic variants, or related sequences.

Probes may also be used for the detection of related sequences, and may have at least 50% sequence identity to any of the NAAP encoding sequences. The hybridization probes of the subject invention may be DNA or RNA and may be derived from the sequence of SEQ ID NO:24-46 or from genomic sequences including promoters, enhancers, and introns of the NAAP gene.

Means for producing specific hybridization probes for DNAs encoding NAAP include the cloning of polynucleotide sequences encoding NAAP or NAAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding NAAP may be used for the diagnosis of disorders associated with expression of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasisectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm. The polynucleotide sequences encoding NAAP may be used in Southern or northern analysis, dot blot, or other membrane-based technologies; in PCR technologies; in dipstick, pin, and multiformat ELISA-like assays; and in microarrays utilizing fluids or tissues from patients to detect altered NAAP expression. Such qualitative or quantitative methods are well known in the art.

In a particular aspect, the nucleotide sequences encoding NAAP may be useful in assays that detect the presence of associated disorders, particularly those mentioned above. The nucleotide sequences encoding NAAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding NAAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of NAAP, a normal or standard profile for expression is established. This may be accomplished by combining body fluids or cell extracts taken from normal subjects, either animal or human, with a sequence, or a fragment thereof, encoding NAAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding NAAP may involve the use of PCR. These oligomers may be chemically synthesized, generated enzymatically, or produced in vitro. Oligomers will preferably contain a fragment of a polynucleotide encoding NAAP, or a fragment of a polynucleotide complementary to the polynucleotide encoding NAAP, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding NAAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from the polynucleotide sequences encoding NAAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641.)

Methods which may also be used to quantify the expression of NAAP include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, NAAP, fragments of NAAP, or antibodies specific for NAAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http:H//www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for NAAP to quantify the levels of NAAP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or aminoreactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding NAAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding NAAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, NAAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between NAAP and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with NAAP, or fragments thereof, and washed. Bound NAAP is then detected by methods well known in the art. Purified NAAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding NAAP specifically compete with a test compound for binding NAAP. In this manner, antibodies can be used to detect the presence of any peptide which shares one or more antigenic determinants with NAAP.

In additional embodiments, the nucleotide sequences which encode NAAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, in particular U.S. Ser. No. 60/288,598, U.S. Ser. No. 60/291,776, U.S. Ser. No. 60/292,172, and U.S. Ser. No. 60/293,564 are expressly incorporated by reference herein.

### EXAMPLES

1. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.16.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 mil of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM-based protein family databases such as PFAM, INCY, and TIGRFAM (Haft, D. H. et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol. 6:361-365.) The queries were performed using programs based on BLAST, FASTA, BLIPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide of the invention may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:24-46. Fragments from about 20 to about 4000 nucleotides which are useful in hybridization and amplification technologies are described in Table 4, column 2.

IV. Identification and Editing of Coding Sequences from Genomic DNA

Putative nucleic acid-associated proteins were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode nucleic acid-associated proteins, the encoded polypeptides were analyzed by querying against PFAM models for nucleic acid-associated proteins. Potential nucleic acid-associated proteins were also identified by homology to Incyte cDNA sequences that had been annotated as nucleic acid-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data “Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of NAAP Encoding Polynucleotides

The sequences which were used to assemble SEQ ID NO:24-46 were compared with sequences from the Incyte LIFESEQ database and public domain databases using BLAST and other implementations of the Smith-Waterman algorithm. Sequences from these databases that matched SEQ ID NO:24-46 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

BLAST Score×Percent Identity

5×minimum {length(Seq. 1), length(Seq. 2)}

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotide sequences encoding NAAP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding NAAP. cDNA sequences and cDNA library/tissue information are found in the LIFESEQ GOLD database (Incyte Genomics, Palo Alto Calif.).

VIII. Extension of NAAP Encoding Polynucleotides

Full length polynucleotide sequences were also produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C. 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene OR) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, full length polynucleotide sequences are verified using the above procedure or are used to obtain 5′ regulatory sequences using the above procedure along with oligonucleotides designed for such extension, and an appropriate genomic library.

IX. Identification of Single Nucleotide Polymorphisms in NAAP Encoding Polynucleotides

Common DNA sequence variants known as single nucleotide polymorphisms (SNPS) were identified in SEQ ID NO:24-46 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:24-46 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X—Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

Expression

For example, expression of SEQ ID NO:35 was shown to be downregulated in liver cell line treated with steroids vs. untreated liver cell line controls. Hepatoblastoma cells were obtained from a 15-year-old male with liver tumor, and used to derive a cell line expressing insulin receptor and insulin-like growth factor II receptor. Samples were treated with steroids including progesterone, betamethasone, dexamethasone, prednisone, budesonide, medroxyprogesterone, and beclomethasone. Therefore, SEQ ID NO:35 may be useful in diagnosis and treatment of autoimmune/inflammatory disorders.

In a further example, SEQ ID NO:41 showed differential expression in preadipocyte tissue treated with PPAR-gamma agonist and differentiation medium versus untreated tissue, as determined by microarray analysis. SEQ ID NO:41 is therefore useful in treatment of metabolic disorders such as diabetes. Primary subcutaneous preadipocytes were isolated from adipose tissue of a 40-year-old female with a body mass index (BMI) of 32.47. The preadipocytes were cultured in differentiation medium containing the active components PPAR-gamma and human insulin (Zen-Bio), to induce differentiation into adipocytes, and subsequently were switched to medium containing insulin alone. Differentiated adipocytes were compared to untreated preadipocytes maintained in culture in the absence of inducing agents.

XII. Complementary Polynucleotides

Sequences complementary to the NAAP-encoding sequences, or any parts thereof, are used to detect, decrease, or inhibit expression of naturally occurring NAAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of NAAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the NAAP-encoding transcript.

XIII. Expression of NAAP

Expression and purification of NAAP is achieved using bacterial or virus-based expression systems. For expression of NAAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express NAAP upon induction with isopropyl beta-D-thiogalactopyranoside (IPTG). Expression of NAAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding NAAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, NAAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Pharmacia Biotech). Following purification, the GST moiety can be proteolytically cleaved from NAAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified NAAP obtained by these methods can be used directly in the assays shown in Examples XVII, XVIII, and XIX, where applicable.

XIV. Functional Assays

NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include PCMV SPORT (Life Technologies) and PCR3.1 (Invitrogen, Carlsbad Calif.), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding NAAP and other genes of interest can be analyzed by northern analysis or microarray techniques.

XV. Production of NAAP Specific Antibodies

NAAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the NAAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Freund's adjuvant. Resulting antisera are tested for antipeptide and anti-NAAP activity by, for example, binding the peptide or NAAP to a substrate, blocking with 1% BSA, reacting with rabbit antisera, washing, and reacting with radio-iodinated goat anti-rabbit IgG.

XVI. Purification of Naturally Occurring NAAP Using Specific Antibodies

Naturally occurring or recombinant NAAP is substantially purified by immunoaffinity chromatography using antibodies specific for NAAP. An immunoaffinity column is constructed by covalently coupling anti-NAAP antibody to an activated chromatographic resin, such as CNBr-activated SEPHAROSE (Amersham Pharmacia Biotech). After the coupling, the resin is blocked and washed according to the manufacturer's instructions.

Media containing NAAP are passed over the immunoaffinity column, and the column is washed under conditions that allow the preferential absorbance of NAAP (e.g., high ionic strength buffers in the presence of detergent). The column is eluted under conditions that disrupt antibody/NAAP binding (e.g., a buffer of pH 2 to pH 3, or a high concentration of a chaotrope, such as urea or thiocyanate ion), and NAAP is collected.

XVII. Identification of Molecules Which Interact with NAAP

NAAP, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled NAAP, washed, and any wells with labeled NAAP complex are assayed. Data obtained using different concentrations of NAAP are used to calculate values for the number, affinity, and association of NAAP with the candidate molecules.

Alternatively, molecules interacting with NAAP are analyzed using the yeast two-hybrid system as described in Fields, S. and O. Song (1989) Nature 340:245-246, or using commercially available kits based on the two-hybrid system, such as the MATCHMAKER system (Clontech).

NAAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of NAAP Activity

NAAP activity is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (Lexₒₚ) fused to sequences encoding the E. coli LacZ enzyme. The methods for constructing and expressing fusion genes, introducing them into cells, and measuring LacZ enzyme activity, are well known to those skilled in the art. Sequences encoding NAAP are cloned into a plasmid that directs the synthesis of a fusion protein, LexA-NAAP, consisting of NAAP and a DNA binding domain derived from the LexA transcription factor. The resulting plasmid, encoding a LexA-NAAP fusion protein, is introduced into yeast cells along with a plasmid containing the LexAₒₚ-LacZ reporter gene. The amount of LacZ enzyme activity associated with LexA-NAAP transfected cells, relative to control cells, is proportional to the amount of transcription stimulated by the NAAP.

Alternatively, NAAP activity is measured by its ability to bind zinc. A 5-10 μM sample solution in 2.5 mM ammonium acetate solution at pH 7.4 is combined with 0.05 M zinc sulfate solution (Aldrich, Milwaukee Wis.) in the presence of 100 μM dithiothreitol with 10% methanol added. The sample and zinc sulfate solutions are allowed to incubate for 20 minutes. The reaction solution is passed through a VYDAC column (Grace Vydac, Hesperia, Calif.) with approximately 300 Angstrom bore size and 5 μM particle size to isolate zinc-sample complex from the solution, and into a mass spectrometer (PE Sciex, Ontario, Canada). Zinc bound to sample is quantified using the functional atomic mass of 63.5 Da observed by Whittal, R. M. et al. ((2000) Biochemistry 39:8406-8417).

In the alternative, a method to determine nucleic acid binding activity of NAAP involves a polyacrylamide gel mobility-shift assay. In preparation for this assay, NAAP is expressed by transforming a mammalian cell line such as COS7, HeLa or CHO with a eukaryotic expression vector containing NAAP cDNA. The cells are incubated for 48-72 hours after transformation under conditions appropriate for the cell line to allow expression and accumulation of NAAP. Extracts containing solubilized proteins can be prepared from cells expressing NAAP by methods well known in the art. Portions of the extract containing NAAP are added to [³²P]-labeled RNA or DNA. Radioactive nucleic acid can be synthesized in vitro by techniques well known in the art. The mixtures are incubated at 25° C. in the presence of RNase- and DNase-inhibitors under buffered conditions for 5-10 minutes. After incubation, the samples are analyzed by polyacrylamide gel electrophoresis followed by autoradiography. The presence of a band on the autoradiogram indicates the formation of a complex between NAAP and the radioactive transcript. A band of similar mobility will not be present in samples prepared using control extracts prepared from untransformed cells.

In the alternative, a method to determine methylase activity of NAAP measures transfer of radiolabeled methyl groups between a donor substrate and an acceptor substrate. Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg NAAP, and acceptor substrate (e.g., 0.4 μg [³⁵S]RNA, or 6-mercaptopurine (6-MP) to 1 mM final concentration). Reaction mixtures are incubated at 30° C. for 30 minutes, then 65° C. for 5 minutes.

Analysis of [methyl-³H]RNA is as follows: (1) 50 μl of 2×loading buffer (20 mM Tris-HCl, pH 7.6, 1 M LiCl, 1 mM EDTA, 1% sodium dodecyl sulphate (SDS)) and 50 μl oligo d(T)-cellulose (10 mg/ml in 1×loading buffer) are added to the reaction mixture, and incubated at ambient temperature with shaking for 30 minutes. (2) Reaction mixtures are transferred to a 96-well filtration plate attached to a vacuum apparatus. (3) Each sample is washed sequentially with three 2.4 ml aliquots of 1×oligo d(T) loading buffer containing 0.5% SDS, 0.1% SDS, or no SDS. (4) RNA is eluted with 300 μl of water into a 96-well collection plate, transferred to scintillation vials containing liquid scintillant, and radioactivity determined.

Analysis of [methyl-³H]6-MP is as follows: (1) 500 μl 0.5 M borate buffer, pH 10.0, and then 2.5 ml of 20% (v/v) isoamyl alcohol in toluene are added to the reaction mixtures. (2) The samples are mixed by vigorous vortexing for ten seconds. (3) After centrifugation at 700 g for 10 minutes, 1.5 ml of the organic phase is transferred to scintillation vials containing 0.5 mfl absolute ethanol and liquid scintillant, and radioactivity determined. (4) Results are corrected for the extraction of 6-MP into the organic phase (approximately 41%).

In the alternative, type I topoisomerase activity of NAAP can be assayed based on the relaxation of a supercoiled DNA substrate. NAAP is incubated with its substrate in a buffer lacking Mg²⁺ and ATP, the reaction is terminated, and the products are loaded on an agarose gel. Altered topoisomers can be distinguished from supercoiled substrate electrophoretically. This assay is specific for type I topoisomerase activity because Mg²⁺ and ATP are necessary cofactors for type II topoisomerases.

Type II topoisomerase activity of NAAP can be assayed based on the decatenation of a kinetoplast DNA (KDNA) substrate. NAAP is incubated with KDNA, the reaction is terminated, and the products are loaded on an agarose gel. Monomeric circular KDNA can be distinguished from catenated KDNA electrophoretically. Kits for measuring type I and type II topoisomerase activities are available commercially from Topogen (Columbus Ohio).

ATP-dependent RNA helicase unwinding activity of NAAP can be measured by the method described by Zhang and Grosse (1994; Biochemistry 33:3906-3912). The substrate for RNA unwinding consists of ³P-labeled RNA composed of two RNA strands of 194 and 130 nucleotides in length containing a duplex region of 17 base-pairs. The RNA substrate is incubated together with ATP, Mg²⁺, and varying amounts of NAAP in a Tris-HCl buffer, pH 7.5, at 37° C. for 30 minutes. The single-stranded RNA product is then separated from the double-stranded RNA substrate by electrophoresis through a 10% SDS-polyacrylamide gel, and quantitated by autoradiography. The amount of single-stranded RNA recovered is proportional to the amount of NAAP in the preparation.

In the alternative, NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64-GFP fusion protein. Flow cytometry (FCM), an automated laser optics-based technique, is used to identify transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Inc., Lake Success N.Y.). mRNA can be purified from the cells using methods well known by those of skill in the art. Expression of mRNA encoding NAAP and other genes of interest can be analyzed by northern analysis or microarray techniques.

Pseudouridine synthase activity of NAAP is assayed using a tritium (³H) release assay modified from Nurse et al. ((1995) RNA 1:102-112), which measures the release of ³H from the C₅ position of the pyriridine component of uridylate (U) when ³H-radiolabeled U in RNA is isomerized to pseudouridine (ψ). A typical 500 μl assay mixture contains 50 mM HEPES buffer (pH 7.5), 100 mM ammonium acetate, 5 mM dithiothreitol, 1 mM EDTA, 30 units RNase inhibitor, and 0.1-4.2 μM [5-³H]tRNA (approximately 1 μCi/nmol tRNA). The reaction is initiated by the addition of <5 μl of a concentrated solution of NAAP (or sample containing NAAP) and incubated for 5 min at 37 ° C. Portions of the reaction mixture are removed at various times (up to 30 min) following the addition of NAAP and quenched by dilution into 1 ml 0.1 M HCl containing Norit-SA3 (12% w/v). The quenched reaction mixtures are centrifuged for 5 min at maximum speed in a microcentrifuge, and the supernatants are filtered through a plug of glass wool. The pellet is washed twice by resuspension in 1 ml 0.1 M HCl, followed by centrifugation. The supernatants from the washes are separately passed through the glass wool plug and combined with the original filtrate. A portion of the combined filtrate is mixed with scintillation fluid (up to 10 ml) and counted using a scintillation counter. The amount of ³H released from the RNA and present in the soluble filtrate is proportional to the amount of peudouridine synthase activity in the sample (Ramamurthy, V. (1999) J. Biol. Chem. 274:22225-22230).

In the alternative, pseudouridine synthase activity of NAAP is assayed at 30 ° C. to 37° C. in a mixture containing 100 mM Tris-HCl (pH 8.0), 100 mM ammonium acetate, 5 mM MgCl₂, 2 mM dithiothreitol, 0.1 mM EDTA, and 1-2 fmol of [³²P]-radiolabeled runoff transcripts (generated in vitro by an appropriate RNA polymerase, i.e., T7 or SP6) as substrates. NAAP is added to initiate the reaction or omitted from the reaction in control samples. Following incubation, the RNA is extracted with phenol-chloroform, precipitated in ethanol, and hydrolyzed completely to 3-nucleotide monophosphates using RNase T₂. The hydrolysates are analyzed by two-dimensional thin layer chromatography, and the amount of ³²P radiolabel present in the ψMP and UMP spots are evaluated after exposing the thin layer chromatography plates to film or a PhosphorImager screen. Taking into account the relative number of uridylate residues in the substrate RNA, the relative amount ψMP and UMP are determined and used to calculate the relative amount of ψ per tRNA molecule (expressed in mol ψ/mol of tRNA or mol ψ/mol of tRNA/minute), which corresponds to the amount of pseudouridine synthase activity in the NAAP sample (Lecointe, F. et al. (1998) J. Biol. Chem. 273:1316-1323).

N²,N²-dimethylguanosine transferase ((m²₂G)methyltransferase) activity of NAAP is measured in a 160 μl reaction mixture containing 100 MM Tris-HCl (pH 7.5), 0.1 mM EDTA, 10 mM MgCl₂, 20 mM NH₄Cl, 1 mM dithiothreitol, 6.2 μM S-adenosyl-L-[methyl-³H]methionine (30-70 Ci/mM), 8 μg m²₂G-deficient tRNA or wild type tRNA from yeast, and approximately 100 μg of purified NAAP or a sample comprising NAAP. The reactions are incubated at 30 ° C. for 90 min and chilled on ice. A portion of each reaction is diluted to 1 ml in water containing 100 μg BSA. 1 ml of 2 M HCl is added to each sample and the acid insoluble products are allowed to precipitate on ice for 20 min before being collected by filtration through glass fiber filters. The collected material is washed several times with HCl and quantitated using a liquid scintillation counter. The amount of ³H incorporated into the m²₂G-deficient, acid-insoluble tRNAs is proportional to the amount of N²,N²-dimethylguanosine transferase activity in the NAAP sample. Reactions comprising no substrate tRNAs, or wild-type tRNAs that have already been modified, serve as control reactions which should not yield acid-insoluble ³H-labeled products.

Polyadenylation activity of NAAP is measured using an in vitro polyadenylation reaction. The reaction mixture is assembled on ice and comprises 10 μl of 5 mM dithiothreitol, 0.025% (v/v) NONIDET P40, 50 mM creatine phosphate, 6.5% (w/v) polyvinyl alcohol, 0.5 unit/μl RNAGUARD (Pharmacia), 0.025 μg/μl creatine kinase, 1.25 mM cordycepin 5′-triphosphate, and 3.75 mM MgCl₂, in a total volume of 25 μl. 60 fmol of CstF, 50 fmol of CPSF, 240 fmol of PAP, 4 μl of crude or partially purified CF II and various amounts of amounts CF I are then added to the reaction mix. The volume is adjusted to 23.5 μl with a buffer containing 50 mM TrisHCl, pH 7.9, 10% (v/v) glycerol, and 0.1 mM Na-EDTA. The final ammonium sulfate concentration should be below 20 mM. The reaction is initiated (on ice) by the addition of 15 fmol of ³²P-labeled pre-mRNA template, along with 2.5 μg of unlabeled tRNA, in 1.5 μl of water. Reactions are then incubated at 30 ° C. for 75-90 min and stopped by the addition of 75 μl (approximately two-volumes) of proteinase K mix (0.2 M Tris-HCl, pH 7.9, 300 mM NaCl, 25 mM Na-EDTA, 2% (w/v) SDS), 1 μl of 10 mg/ml proteinase K, 0.25 μl of 20 mg/ml glycogen, and 23.75 μl of water). Following incubation, the RNA is precipitated with ethanol and analyzed on a 6% (w/v) polyacrylamide, 8.3 M urea sequencing gel. The dried gel is developed by autoradiography or using a phosphoimager. Cleavage activity is determined by comparing the amount of cleavage product to the amount of pre-mRNA template. The omission of any of the polypeptide components of the reaction and substitution of NAAP is useful for identifying the specific biological function of NAAP in pre-mRNA polyadenylation (Ruegsegger, U. et al. (1996) J. Biol. Chem. 271:6107-6113; and references within).

tRNA synthetase activity is measured as the aminoacylation of a substrate tRNA in the presence of [¹⁴C]-labeled amino acid. NAAP is incubated with [¹⁴C]-labeled amino acid and the appropriate cognate tRNA (for example, [¹⁴C]alanine and tRNA^{ala}) in a buffered solution. ¹⁴C-labeled product is separated from free [¹⁴C]amino acid by chromatography, and the incorporated ¹⁴C is quantified by scintillation counter. The amount of ¹⁴C-labeled product detected is proportional to the activity of NAAP in this assay.

In the alternative, NAAP activity is measured by incubating a sample containing NAAP in a solution containing 1 mM ATP, 5 mM Hepes-KOH (pH 7.0), 2.5 mM KCl, 1.5 mM magnesium chloride, and 0.5 mM DTT along with misacylated [¹⁴C]-Glu-tRNAGln (e.g., 1 μM) and a similar concentration of unlabeled L-glutamine. Following the quenching of the reaction with 3 M sodium acetate (pH 5.0), the mixture is extracted with an equal volume of water-saturated phenol, and the aqueous and organic phases are separated by centrifugation at 15,000×g at room temperature for 1 min. The aqueous phase is removed and precipitated with 3 volumes of ethanol at −70° C. for 15 min. The precipitated aminoacyl-tRNAs are recovered by centrifugation at 15,000×g at 4° C. for 15 min. The pellet is resuspended in of 25 mM KOH, deacylated at 65° C. for 10 min., neutralized with 0.1 M HCl (to final pH 6-7), and dried under vacuum. The dried pellet is resuspended in water and spotted onto a cellulose TLC plate. The plate is developed in either isopropanol/formic acid/water or ammonia/water/chloroform/ methanol. The image is subjected to densitometric analysis and the relative amounts of Glu and Gln are calculated based on the Rf values and relative intensities of the spots. NAAP activity is calculated based on the amount of Gln resulting from the transformation of Glu while acylated as Glu-tRNA^{Gln} (adapted from Curnow, A. W. et al. (1997) Proc. Natl. Acad. Sci. USA 94:11819-26).

Alternatively, NAAP activity is demonstrated by increase in chromatin activity. Chromatin activity is proportional to sensitivity to DNase I (Dawson, B. A. et al. (1989) J. Biol. Chem. 264:12830-12837). NAAP-containing sample (NAAP+) and a control sample (NAAP−) are treated with DNase I, followed by insertion of a cleavable biotinylated nucleotide analog, 5-[(N-biotinamido)hexanoamido-ethyl-1,3-thiopropionyl-3-aminoallyl]-2′-deoxyuridine 5′-triphosphate, using nick-repair techniques well known to those skilled in the art. Following purification and digestion with EcoRI restriction endonuclease, biotinylated sequences are affinity isolated by sequential binding to streptavidin and biotincellulose. The difference in biotinylation in the presence and absence of NAAP is proportional to NAAP activity.

XIX. Identification of NAAP Agonists and Antagonists

Agonists or antagonists of NAAP activation or inhibition may be tested using the assays described in section XVIII. Agonists cause an increase in NAAP activity and antagonists cause a decrease in NAAP activity.

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| | | | | Incyte |
|---|---|---|---|---|
| | | Incyte | Poly- | Poly- |
| Incyte | Poly- | Poly- | nucleo- | nucleo- |
| Project | peptide | peptide | tide | tide |
| ID | SEQ ID NO: | ID | SEQ ID NO: | ID |
| 4936875 | 1 | 4936875CD1 | 24 | 4936875CB1 |
| 264408 | 2 | 264408CD1 | 25 | 264408CB1 |
| 2181434 | 3 | 2181434CD1 | 26 | 2181434CB1 |
| 1367252 | 4 | 1367252CD1 | 27 | 1367252CB1 |
| 5633694 | 5 | 5633694CD1 | 28 | 5633694CB1 |
| 7985981 | 6 | 7985981CD1 | 29 | 7985981CB1 |
| 4706628 | 7 | 4706628CD1 | 30 | 4706628CB1 |
| 5790110 | 8 | 5790110CD1 | 31 | 5790110CB1 |
| 2948827 | 9 | 2948827CD1 | 32 | 2948827CB1 |
| 1398040 | 10 | 1398040CD1 | 33 | 1398040CB1 |
| 7716061 | 11 | 7716061CD1 | 34 | 7716061CB1 |
| 6113748 | 12 | 6113748CD1 | 35 | 6113748CB1 |
| 7474037 | 13 | 7474037CD1 | 36 | 7474037CB1 |
| 2955646 | 14 | 2955646CD1 | 37 | 2955646CB1 |
| 1573006 | 15 | 1573006CD1 | 38 | 1573006CB1 |
| 1336756 | 16 | 1336756CD1 | 39 | 1336756CB1 |
| 71259816 | 17 | 71259816CD1 | 40 | 71259816CB1 |
| 3354130 | 18 | 3354130CD1 | 41 | 3354130CB1 |
| 1797985 | 19 | 1797985CD1 | 42 | 1797985CB1 |
| 2870383 | 20 | 2870383CD1 | 43 | 2870383CB1 |
| 1285088 | 21 | 1285088CD1 | 44 | 1285088CB1 |
| 1532441 | 22 | 1532441CD1 | 45 | 1532441CB1 |
| 3056408 | 23 | 3056408CD1 | 46 | 3056408CB1 |

TABLE 2

| Poly- | Incyte | GenBank ID | | |
|---|---|---|---|---|
| peptide | Poly- | NO:or | | |
| SEQ ID | peptide | PROTEOME | Probability | |
| NO: | ID | ID NO: | Score | Annotation |
| 1 | 4936875CD1 | g3255965 | 0 | [Homo sapiens] |
| | | | | U5 snRNP-specific |
| | | | | 200 kD protein Lauber, |
| | | | | J. et al. (1996) |
| | | | | EMBO J. 15: 4001-4015 |
| 2 | 264408CD1 | g4510377 | 1.90E−168 | [Arabidopsis thaliana] |
| | | | | putative ATP-dependent RNA |
| | | | | helicase A |
| 3 | 2181434CD1 | g6901197 | 3.50E−48 | [Schizosaccharomyces pombe] |
| | | | | putative helicase |
| 4 | 1367252CD1 | g13699244 | 0 | [Homo sapiens] CLLL8 |
| | | | | protein Mabuchi H, et al. |
| | | | | (2001) Cancer Res. 61: |
| | | | | 2870-2877 |
| 5 | 5633694CD1 | g4730929 | 1.40E−140 | [Homo sapiens] |
| | | | | HCF-binding transcription |
| | | | | factor Zhangfei Lu, R. |
| | | | | and Misra. V. (2000) |
| | | | | Nucleic Acids |
| | | | | Res. 28, 2446-2454 |
| 6 | 7985981CD1 | g2429354 | 2.50E−45 | [Mus musculus] |
| | | | | EWS/FLI1 activated |
| | | | | transcript 2 Thompson, A. D. |
| | | | | et al. (1996) Oncogene 13: |
| | | | | 2649-2658 |
| 7 | 4706628CD1 | g4220590 | 6.90E−224 | [Mus musculus] nuclear |
| | | | | protein np95 Fujimori, A. et al. |
| | | | | (1998) Mamm. Genome 9: |
| | | | | 1032-1035 |
| 8 | 5790110CD1 | g14349166 | 0 | [Homo sapiens] |
| | | | | Werner helicase |
| | | | | interacting protein |
| | | | | Kawabe, Y., et al. |
| | | | | (2001) J. Biol. Chem. 276: |
| | | | | 20364-20369 |
| 9 | 2948827CD1 | g1885356 | 1.80E−49 | [Homo sapiens] |
| | | | | type 1 RNA |
| | | | | helicase pNORF1 |
| | | | | Applequist, S. E. |
| | | | | et al. (1997) Nucleic |
| | | | | Acids Res. 25: |
| | | | | 814-821 |
| 10 | 1398040CD1 | g10121865 | 6.90E−24 | [Homo sapiens] |
| | | | | topoisomerase II |
| | | | | alpha-4 Petruti-Mot, |
| | | | | A. S., Earnshaw, W. C. |
| | | | | (2000)Gene 258: |
| | | | | 183-192 |
| 11 | 7716061CD1 | g10121865 | 5.10E−23 | [Homo sapiens] |
| | | | | topoisomerase II |
| | | | | alpha-4 Petruti-Mot, |
| | | | | A. S., Earnshaw, W. C. |
| | | | | (2000)Gene 258: |
| | | | | 183-192 |
| 12 | 6113748CD1 | g1770528 | 6.30E−15 | [Homo sapiens] |
| | | | | Translin Associated |
| | | | | Zinc Finger protein- |
| | | | | 1 Aoki, K. et al. |
| | | | | (1997) FEBS Lett. |
| | | | | 401: 109-112 |
| 13 | 7474037CD1 | g12655063 | 7.00E−66 | [Homo sapiens] |
| | | | | (BC001381) polymerase |
| | | | | (RNA) III (DNA directed) |
| | | | | polypeptide K (12.3 kDa) |
| 14 | 2955646CD1 | g409139 | 1.40E−156 | [Homo sapiens] |
| | | | | paired-box protein |
| | | | | Eccles, M. R. et al. (1992) |
| | | | | Cell Growth Differ. |
| | | | | 3: 279-289 |
| 15 | 1573006CD1 | g487787 | 1.60E−63 | [Homo sapiens] |
| | | | | zinc finger protein |
| | | | | ZNF140 Vissing, H. et al. |
| | | | | (1995) FEBS Lett. |
| | | | | 369: 153-157 |
| 16 | 1336756CD1 | g3638956 | 1.90E−294 | [Homo sapiens] |
| | | | | zinc finger-like; |
| | | | | similar to P52742 |
| | | | | (PID: g1731411) |
| 18 | 3354130CD1 | g2306773 | 3.90E−90 | [Homo sapiens] |
| | | | | zinc finger protein |
| | | | | Lee, P. L. et al. (1997) |
| | | | | Genomics 43: 191-201 |
| 19 | 1797985CD1 | g14134120 | 0 | [Caenorhabditis elegans] |
| | | | | endocytosis protein |
| | | | | RME-8 Zhang, Y., et al. |
| | | | | (2001) Mol. Biol. Cell |
| | | | | 12: 2011-2021 |
| 20 | 2870383CD1 | g7021370 | 8.90E−100 | [Drosophila melanogaster] |
| | | | | c12.2 Batterham, P., et al. |
| | | | | (2000) Molecular structure |
| | | | | of the lozenge gene of |
| | | | | Drosophila melanogaster, |
| | | | | Accession: AAF35310 |
| 21 | 1285088CD1 | g6016005 | 3.50E−256 | [Homo sapiens] |
| | | | | CoREST protein |
| | | | | Andres, M. E. et al. |
| | | | | (1999) Proc. Natl. |
| | | | | Acad. Sci. U.S.A. |
| | | | | 96: 9873-9878 |
| 22 | 1532441CD1 | g11094232 | 9.40E−23 | [Mus musculus] |
| | | | | neural activity- |
| | | | | related ring finger |
| | | | | protein Ohkawa, N., |
| | | | | et al. (2001) J. |
| | | | | Neurochem. 78: |
| | | | | 75-87 |
| 23 | 3056408CD1 | g11527189 | 0 | [Homo sapiens] |
| | | | | p250R Kato, H., |
| | | | | et al. (2002) J. |
| | | | | Biol. Chem. |
| | | | | 277: 5498-5505 |

TABLE 3

| | | | | | | Analytical |
|---|---|---|---|---|---|---|
| | Incyte | Amino | Potential | Potential | | Methods |
| SEQ ID | Polypeptide | Acid | Phosphorylation | Glycosylation | Signature Sequences, | and |
| NO: | ID | Residues | Sites | Sites | Domains and Motifs | Databases |
| 1 | 4936875CD1 | 2136 | S26 S207 S225 | N995 N1026 N1531 | DEAD/DEAH box helicase: | HMMER-PFAM |
| | | | | | A471-F676, E1318- | |
| | | | | | Q1528 | |
| | | | S252 S253 S282 | N1825 N2103 | | |
| | | | S339 S377 S385 | | | |
| | | | S434 S679 S727 | | | |
| | | | S872 S939 S1010 | | | |
| | | | S1051 S1056 S1272 | | | |
| | | | S1285 S1315 S1354 | | | |
| | | | S1427 S1436 S1479 | | | |
| | | | S1554 S1565 S1625 | | | |
| | | | S1709 S1726 S1777 | | | |
| | | | S1794 SI803 S1910 | | | |
| | | | S1981 S2028 T31 | | | |
| | | | S2078 S2133 T5 | | | |
| | | | T180 T295 T366 | | | |
| | | | T389 T565 T597 | | | |
| | | | T642 T693 T731 | | | |
| | | | T734 T764 T790 | | | |
| | | | T795 T863 T1008 | | | |
| | | | T1028 T1108 | | | |
| | | | T1197 T1380 | | | |
| | | | T1412 T1569 | | | |
| | | | T1572 T1593 | | | |
| | | | T1608 T1765 | | | |
| | | | T1792 T1827 | | | |
| | | | T1975 T2083 | | | |
| | | | T2131 Y605 | | | |
| | | | | | Helicase conserved C- | HMMER-PFAM |
| | | | | | terminal domain: | |
| | | | | | Q768-Q860 | |
| | | | | | Helicase, ATP-binding, | BLAST-PRODOM |
| | | | | | nuclear protein, pre- | |
| | | | | | mRNA splicing, BRR2 | |
| | | | | | PD007814: I1425- | |
| | | | | | N1449, K1711-K2089, | |
| | | | | | P859-L1190, P1694- | |
| | | | | | L1904, H1965-Y2113 | |
| | | | | | pre-mRNA splicing heli- | BLAST-PRODOM |
| | | | | | case BRR2 EC 3.6.1. | |
| | | | | | PD184330: L1262-S1492 | |
| | | | | | pre-mRNA splicing heli- | BLAST-PRODOM |
| | | | | | case BRR2 PD043126: | |
| | | | | | R270-F475, Y11- | |
| | | | | | D228, R25-F475, | |
| | | | | | L1609-A1657 | |
| | | | | | Nucleolar helicase | BLAST-DOMO |
| | | | | | SKI2W, SKI2 DM01537: | |
| | | | | | P32639\|502-912: | |
| | | | | | I484-V894, 11331- | |
| | | | | | S1726 | |
| | | | | | P53327\|279-707: | |
| | | | | | F478-Q892, L1369- | |
| | | | | | S1726, F1325-T1569, | |
| | | | | | R1133-F1150 | |
| | | | | | P53327\|1130-1542: | |
| | | | | | E1318-L1728, F478- | |
| | | | | | M893 | |
| | | | | | ATP/GTP-binding site | MOTIFS |
| | | | | | motif A (P-loop): | |
| | | | | | A503-T510, A1350- | |
| | | | | | T1357 | |
| 2 | 264408CD1 | 1386 | S4 S19 S54 S70 | N80 N460 N493 | DEAD/DEAH box helicase: | HMMER-PFAM |
| | | | S74 S82 S127 S132 | N766 | R612-E706 | |
| | | | S240 S293 S337 | | | |
| | | | S376 S475 S480 | | | |
| | | | S504 S605 S608 | | | |
| | | | S633 S689 S752 | | | |
| | | | S788 S819 S878 | | | |
| | | | S891 S931 S947 | | | |
| | | | S1032 S1099 S1134 | | | |
| | | | S1152 S1161 S1205 | | | |
| | | | S1325 T196 T225 | | | |
| | | | T282 T399 T413 | | | |
| | | | T768 T921 T1047 | | | |
| | | | T1199 T1256 | | | |
| | | | T1289 Y365 Y939 | | | |
| | | | Y1280 | | | |
| | | | | | Zinc finger | HMMER-PFAM |
| | | | | | C-x8-C-x5-C-x3-H type: | |
| | | | | | N300-V325 | |
| | | | | | Transmembrane domain: | TMAP |
| | | | | | G841-Y864, P1063- | |
| | | | | | L1091, I1294-G1309 | |
| | | | | | N-terminus is non- | |
| | | | | | cytosolic | |
| | | | | | DEAH-box subfamily | BLIMPS-BLOCKS |
| | | | | | BL00690: T599-E616, | |
| | | | | | 1665-T674, G567- | |
| | | | | | Q576 | |
| | | | | | DEAD and DEAH box | ProfileScan |
| | | | | | families ATP-dependent | |
| | | | | | helicases signatures: | |
| | | | | | L642-P692 | |
| | | | | | Zinc finger | BLIMPS-PFAM |
| | | | | | C-x8-C-x5-C-x3-H | |
| | | | | | PF00642: C313-H323 | |
| | | | | | Helicase, nuclear | BLAST-PRODOM |
| | | | | | envelope, ATP-binding | |
| | | | | | PD000440: L857-S979, | |
| | | | | | P545-T702, P903- | |
| | | | | | H980, T180-V210 | |
| | | | | | RNA helicase, ATP- | BLAST-PRODOM |
| | | | | | binding PD001259: | |
| | | | | | C973-Y1121 | |
| | | | | | Helicase PD091835: | BLAST-PRODOM |
| | | | | | Q561-D728, H843- | |
| | | | | | A897 | |
| | | | | | DEAH-box subfamily | BLAST-DOMO |
| | | | | | ATP-dependent helicases | |
| | | | | | DM00649: | |
| | | | | | P24785\|374-1061: | |
| | | | | | T768-T1199, F534- | |
| | | | | | K760, Q985-S1249, | |
| | | | | | K1253-V1292 | |
| | | | | | Q08211\|378-1053: | |
| | | | | | T817-D1197, Q535- | |
| | | | | | S788, S1119-V1292 | |
| | | | | | P34498\|432-1038: | |
| | | | | | G848-I1173, Q535- | |
| | | | | | F730, I1264-F1293, | |
| | | | | | S1205-A1250, | |
| | | | | | V1333-I1364, K762- | |
| | | | | | T817 | |
| | | | | | S59384\|595-1296: | |
| | | | | | K825-F1175, R541- | |
| | | | | | A737, Y1145-Y1261, | |
| | | | | | F285-F359 | |
| | | | | | ATP/GTP-binding site | MOTIFS |
| | | | | | motif A (P-loop): | |
| | | | | | G567-T574 | |
| | | | | | DEAH-box subfamily | MOTIFS |
| | | | | | ATP-dependent helicases | |
| | | | | | signature: S663-E672 | |
| 3 | 2181434CD1 | 604 | S62 S117 S205 | N386 | Transmembrane domain: | TMAP |
| | | | S236 S259 S281 | | P227-M255 | |
| | | | S460 S564 T25 T57 | | N-terminus is non- | |
| | | | T63 T100 T171 | | cytosolic | |
| | | | T290 T445 T478 | | | |
| | | | T534 T586 | | | |
| | | | | | Hypothetical helicase | BLAST-PRODOM |
| | | | | | C28H8.3 in chromosome III, | |
| | | | | | ATP-binding, nuclear | |
| | | | | | PD135267: R211-M431, | |
| | | | | | K510-E593 | |
| | | | | | Nucleolar helicase SKI2W, SKI2 | BLAST-DOMO |
| | | | | | DM01537: | |
| | | | | | A56003\|60-514: | |
| | | | | | D120-F244 | |
| | | | | | S56752\|289-744: | |
| | | | | | D120-F244 | |
| | | | | | P47047\|J131-583: | |
| | | | | | L93-V219 | |
| | | | | | P35207\|J309-803: | |
| | | | | | E82-F244 | |
| 4 | 1367252CD1 | 707 | S34 S67 S94 S111 | N63 N81 N127 | SET domain: V348-T687 | HMMER_PFAM |
| | | | S178 S186 S230 | N209 N269 N272 | | |
| | | | S251 S310 S385 | N467 N609N639 | | |
| | | | S415 S425 T45 T75 | | | |
| | | | T89 T299 T394 | | | |
| | | | T409 T442 T445 | | | |
| | | | T463 T503 T566 | | | |
| | | | T610 T667 T687 | | | |
| | | | Y196 Y398 | | | |
| | | | | | SET domain proteins. | BLIMPS_PFAM |
| | | | | | PF00856: G366-E402, | |
| | | | | | L626-L647 | |
| | | | | | PROTEIN TRANSCRIPTION | BLAST_PRODOM |
| | | | | | REGULATION NUCLEAR DNA | |
| | | | | | BINDING HOMOLOG | |
| | | | | | ENHANCER OF ZESTE SUVAR39 | |
| | | | | | PD001211: | |
| | | | | | F624-E684, R347- | |
| | | | | | K396 | |
| | | | | | PROTEIN SUVAR39 G9A | BLAST_PRODOM |
| | | | | | HOMOLOG PUTATIVE G9A | |
| | | | | | LIKE CLR4P CLR4 ERG | |
| | | | | | ASSOCIATED ESET | |
| | | | | | PD036912: V232-N346 | |
| | | | | | PROTEIN ERG ASSOCIATED | BLAST_PRODOM |
| | | | | | ESET KIAA0067 | |
| | | | | | PD130488: L128-K226 | |
| | | | | | SET DOMAIN | BLAST_DOMO |
| | | | | | DM01286\|S30385\| | |
| | | | | | 716-969: D233- | |
| | | | | | D406, E602-R703 | |
| | | | | | DM01286\|S44861\| | |
| | | | | | 920-1138: V241- | |
| | | | | | S390 | |
| | | | | | DM01286\|P45975\| | |
| | | | | | 370-633: C281-R405, | |
| | | | | | F624-K704 | |
| | | | | | DM01286\|S44861\| | |
| | | | | | 1139-1275: V623- | |
| | | | | | Y683 | |
| 5 | 5633694CD1 | 358 | S79 S103 S175 | N273 | signal_cleavage: M1- | SPSCAN |
| | | | S189 S198 S209 | | S20 | |
| | | | S349 S351 T18 | | | |
| | | | | | bZIP transcription | HMMER_PFAM |
| | | | | | factor: A217-Y266 | |
| | | | | | Leucine zipper pattern | MOTIFS |
| | | | | | L232-L253 L239- | |
| | | | | | L260 L246-L267 | |
| 6 | 7985981CD1 | 132 | S45 S71 S78 S84 | | signal_cleavage: M1- | SPSCAN |
| | | | S105 S107 S123 | | T52 | |
| | | | T11 T52 Y62 | | | |
| | | | | | Src homology domain 2: | HMMER_PFAM |
| | | | | | Y5-F86 | |
| | | | | | Transmembrane domain: | TMAP |
| | | | | | P37-T52 | |
| | | | | | N-terminus is cytosolic | |
| | | | | | SH2 domain signature | BLIMPS_PRINTS |
| | | | | | PR00401: Y5-L19, | |
| | | | | | D25-S35, P37-N48, | |
| | | | | | K59-E69, V75-P89 | |
| 7 | 4706628CD1 | 802 | S20 S114 S170 | N167 | PHD-finger: | HMMER_PFAM |
| | | | S196 S301 S317 | | S346-D395 | |
| | | | S346 S391 S409 | | | |
| | | | S422 S567 S574 | | | |
| | | | S628 S643 S654 | | | |
| | | | S667 S760 T15 T24 | | | |
| | | | T57 T85 T186 | | | |
| | | | T270 T277 T293 | | | |
| | | | T458 T661 T662 | | | |
| | | | T789 Y56 Y386 | | | |
| | | | Y487 Y507 | | | |
| | | | | | Ubiquitin family: | HMMER_PFAM |
| | | | | | M1-T83 | |
| | | | | | ZINC FINGER PROTEIN | BLAST_PRODOM |
| | | | | | PUTATIVE T15F16.7 | |
| | | | | | PD126626: V442-G509 | |
| | | | | | Cell attachment | MOTIFS |
| | | | | | sequence: R501-D503 | |
| | | | | | Zinc finger, C3HC4 type | MOTIFS |
| | | | | | (RING finger), signature: | |
| | | | | | C748-L757 | |
| 8 | 5790110CD1 | 665 | S4 S34 S54 S75 | N334 N415 | ATPase family associated | HMMER_PFAM |
| | | | S92 S139 S153 | N516 | with various cellular | |
| | | | S156 S254 S285 | | activities: S263-A433 | |
| | | | S289 S336 S403 | | | |
| | | | S416 S436 S456 | | | |
| | | | S457 S509 T87 | | | |
| | | | T230 T235 T323 | | | |
| | | | T477 Y434 Y500 | | | |
| | | | Y631 | | | |
| | | | | | Transmembrane domain: | TMAP |
| | | | | | V376-I398; | |
| | | | | | N-terminus is cytosolic | |
| | | | | | PROTEIN ATP-BINDING | BLAST_PRODOM |
| | | | | | INTERGENIC REGION | |
| | | | | | ATP-DEPENDENT PROTEASE | |
| | | | | | LA HOMOLOG HYDROLASE | |
| | | | | | SERINE PD006874: | |
| | | | | | 1424-N598, Q337- | |
| | | | | | A450 | |
| | | | | | PROTEIN ATP-BINDING | BLAST_PRODOM |
| | | | | | INTERGENIC REGION | |
| | | | | | PD150113: V614-K661 | |
| | | | | | Helicase Holliday | BLAST_PRODOM |
| | | | | | junction DNA RUVB | |
| | | | | | repair SOS response ATP | |
| | | | | | Binding Recombination | |
| | | | | | PD003018: L264-N334, | |
| | | | | | Pvalue 2.6e−06 | |
| | | | | | HI1590; SER; SPOIIIE; | BLAST_DOMO |
| | | | | | 49.9; DM03120 | |
| | | | | | P40151\|285-586: | |
| | | | | | L435-L654 | |
| | | | | | S43134\|49-353: | |
| | | | | | L410-F660, | |
| | | | | | N359-P406 | |
| | | | | | P39918\|151-445: | |
| | | | | | L410-F660, | |
| | | | | | L368-P406 | |
| | | | | | P45262\|153-445: | |
| | | | | | M422-F660, | |
| | | | | | L368-I393 | |
| | | | | | Leucine zipper pattern | MOTIFS |
| | | | | | L604-L625 | |
| | | | | | ATP/GTP-binding site | MOTIFS |
| | | | | | motif A (P-loop): | |
| | | | | | G268-T275 | |
| 9 | 2948827CD1 | 677 | S42 S51 S80 S146 | N110 N162 N313 | Viral (Superfamily 1) | HMMER_PFAM |
| | | | S151 S164 S208 | N349 | RNA helicase: T223- | |
| | | | S294 S311 S549 | | L237, I396-P414 | |
| | | | S564 S592 S666 | | | |
| | | | T23 T117 T165 | | | |
| | | | T175 T330 T332 | | | |
| | | | T348 T431 T474 | | | |
| | | | T514 | | | |
| | | | | | Transmembrane domain: | TMAP |
| | | | | | D63-S80 F220-F248 | |
| | | | | | K263-R291 E510- | |
| | | | | | T535 | |
| | | | | | N-terminus is non- | |
| | | | | | cytosolic | |
| | | | | | UvrD/REP helicase. | BLIMPS_PFAM |
| | | | | | PF00580: V561-L579, | |
| | | | | | D591-G603, 1224- | |
| | | | | | V245, K375-T388, | |
| | | | | | V407-S420 | |
| | | | | | PROTEIN HELICASE ATP- | BLAST_PRODOM |
| | | | | | BINDING DNA-BINDING | |
| | | | | | NUCLEAR DNA RNA- | |
| | | | | | DIRECTED RNA POLYMERASE | |
| | | | | | PUTATIVE PD002062: | |
| | | | | | V358-S479 | |
| | | | | | PROTEIN HELICASE ATP- | BLAST_PRODOM |
| | | | | | BINDING DNA-BINDING | |
| | | | | | NUCLEAR RNA-DIRECTED | |
| | | | | | RNA POLYMERASE DNA | |
| | | | | | CHROMOSOME PD001429: | |
| | | | | | K516-N619 | |
| | | | | | RETICULUM; TARGETING | BLAST_DOMO |
| | | | | | DM01082 | |
| | | | | | Q09820\|551-842: | |
| | | | | | T362-E630 | |
| | | | | | S62476\|551-842: | |
| | | | | | T362-E630 | |
| | | | | | P30771\|585-840: | |
| | | | | | P395-G635 | |
| | | | | | Q00416\|1474-1740: | |
| | | | | | Q386-R632 | |
| | | | | | ATP/GTP-binding site | MOTIFS |
| | | | | | motif A (P-loop) | |
| | | | | | G227-S234 | |
| 10 | 1398040CD1 | 107 | S16 S30 S91 T43 | | Signal_cleavage: M1- | SPSCAN |
| | | | T80 | | L19 | |
| | | | | | Transmembrane domain: | TMAP |
| | | | | | A47-H70; | |
| | | | | | N-terminus is cytosolic | |
| | | | | | PROTEIN PROTO-ONCOGENE | BLAST_PRODOM |
| | | | | | NUCLEAR UBIQUITOUS | |
| | | | | | TPR MOTIF Y ISOFORM | |
| | | | | | MYB CMYB PD015557: | |
| | | | | | F60-A101 | |
| 11 | 7716061CD1 | 96 | T65 | | Signal_cleavage: | SPSCAN |
| | | | | | M1-A34 | |
| | | | | | Signal Peptide: | HMMER |
| | | | | | M1-A18 | |
| | | | | | Transmembrane domain: | TMAP |
| | | | | | G39-L61, | |
| | | | | | N-terminus is cytosolic | |
| | | | | | PROTEIN PROTO-ON | BLAST_PRODOM |
| | | | | | COGENE NUCLEAR | |
| | | | | | UBIQUITOUS TPR MOTIF Y | |
| | | | | | ISOFORM MYB CMYB | |
| | | | | | PD015557: F51-A92 | |
| 12 | 6113748CD1 | 469 | S67 S95 S108 S159 | | Signal_cleavage: | SPSCAN |
| | | | S196 S229 S345 | | M1-L53 | |
| | | | S397 T51 T80 | | | |
| | | | T136 T192 T202 | | | |
| | | | T208 T221 T228 | | | |
| | | | T287 | | | |
| | | | | | BTB/POZ domain: | HMMER_PFAM |
| | | | | | R8-V117 | |
| | | | | | Transmembrane domain: | TMAP |
| | | | | | I28-T51; | |
| | | | | | N-terminus is cytosolic | |
| | | | | | Protein DNA binding zinc | BLAST_PRODOM |
| | | | | | finger metal binding | |
| | | | | | PD000632: P4-V105 | |
| | | | | | Pvalue 1.7e−08 | |
| | | | | | POZ DOMAIN DM00509 | BLAST_DOMO |
| | | | | | S59069\|1-171: | |
| | | | | | M1-E174 | |
| | | | | | S44264\|27-229: | |
| | | | | | M1-G123 | |
| | | | | | P24278\|1-212: | |
| | | | | | M1-P84 | |
| 13 | 7474037CD1 | 132 | S100 T25 T42 T62 | N5 N89 N118 N360 | UBIQUITIN DM00160\| | BLAST_DOMO |
| | | | | | S55243\|154-235: | |
| | | | | | G102-R130 | |
| 14 | 2955646CD1 | 332 | S81 S200 S223 | N130 N299 | Signal_cleavage: | SPSCAN |
| | | | S227 S253 S315 | | M1-G22 | |
| | | | T96 T292 T310 | | | |
| | | | Y254 | | | |
| | | | | | Signal Peptide: | HMMER |
| | | | | | M1-G24 | |
| | | | | | ‘Paired box’ | HMMER_PFAM |
| | | | | | domain: G20-R144 | |
| | | | | | Transmembrane domains: | TMAP |
| | | | | | P4-G20; | |
| | | | | | N-terminus non-cytosolic | |
| | | | | | ‘Paired box’ | BLIMPS_BLOCKS |
| | | | | | domain protein BL00034: | |
| | | | | | S175-P185, G20- | |
| | | | | | S70, G74-N110, | |
| | | | | | F114-R144 | |
| | | | | | ‘Paired box’ | PROFILESCAN |
| | | | | | domain signature | |
| | | | | | paired_box.prf: | |
| | | | | | G34-S90 | |
| | | | | | Paired box signature | BLIMPS_PRINTS |
| | | | | | PR00027: V24-D39, | |
| | | | | | R42-R60, L62-T79, | |
| | | | | | G80-P97 | |
| | | | | | PAIRED BOX NUCLEAR | BLAST_PRODOM |
| | | | | | DNA-BINDING | |
| | | | | | PD000643: G20-R144 | |
| | | | | | PD072729: P217-N293 | |
| | | | | | PD004047: P217-N293 | |
| | | | | | PD010666: T145-P176 | |
| | | | | | PAIRED BOX DM00579 | BLAST_DOMO |
| | | | | | Q02962\|13-126: | |
| | | | | | G20-D131 | |
| | | | | | S36156\|12-125: | |
| | | | | | H21-D131 | |
| | | | | | Q02548\|13-126: | |
| | | | | | S17-D131 | |
| | | | | | Q02650\|13-126: | |
| | | | | | G20-D131 | |
| | | | | | ‘Paired box’ | MOTIFS |
| | | | | | domain signature | |
| | | | | | R54-S70 | |
| 15 | 1573006CD1 | 304 | S16 S57 S82 S118 | N38 N270 N296 | KRAB box: V6-D69 | HMMER_PFAM |
| | | | S167 S208 T86 | | | |
| | | | T101 T110 T146 | | | |
| | | | T180 T223 T279 | | | |
| | | | | | Zinc finger, C2H2 type: | HMMER_PFAM |
| | | | | | Y256-H278, Y200- | |
| | | | | | H222, F228-H250 | |
| | | | | | Zinc finger C2H2 type | BLIMPS_BLOCKS |
| | | | | | BL00028: C258-H274 | |
| | | | | | C2H2 type Zinc finger | BLIMPS_PRINTS |
| | | | | | PR00048: P255-G268, | |
| | | | | | D243-G252 | |
| | | | | | PROTEIN ZINC | BLIMPS_PRODOM |
| | | | | | FINGER | |
| | | | | | ZINC PD01066: | |
| | | | | | F8-A46 | |
| | | | | | ZINC FINGER METAL | BLAST_PRODOM |
| | | | | | BINDING DNA-BINDING | |
| | | | | | PROTEIN FINGER | |
| | | | | | ZINC NUCLEAR REPEAT | |
| | | | | | TRANSCRIPTION REGULATION | |
| | | | | | PD001562: V6-D69 | |
| | | | | | PD053122: M106-S154, | |
| | | | | | R245-K254, R273- | |
| | | | | | K281, P212-K226 | |
| | | | | | PD000072: K198-C261, | |
| | | | | | K226-K281 | |
| | | | | | PD017719: S185-C261, | |
| | | | | | D113-T294 | |
| | | | | | KRAB BOX DOMAIN DM00605 | BLAST_DOMO |
| | | | | | I48208\|18-93: | |
| | | | | | S5-W77 | |
| | | | | | S42077\|18-93: | |
| | | | | | S5-W77 | |
| | | | | | P52738\|3-77: | |
| | | | | | Q3-R74 | |
| | | | | | 148689\|11-85: | |
| | | | | | Q3-R74 | |
| | | | | | Zinc finger, C2H2 type, | MOTIFS |
| | | | | | domain: C202-H222 | |
| | | | | | C230-H250 C258- | |
| | | | | | H278 | |
| 16 | 1336756CD1 | 595 | S39 S40 S50 S209 | | Signal_cleavage: | SPSCAN |
| | | | S219 S226 S254 | | M1-G16 | |
| | | | S497 S525 S553 | | | |
| | | | T56 T174 T198 | | | |
| | | | T275 T285 T310 | | | |
| | | | T462 | | | |
| | | | | | Zinc finger, C2H2 type: | HMMER_PFAM |
| | | | | | F459-H481, Y160- | |
| | | | | | H182, F431-H453, | |
| | | | | | F543-H565, F216- | |
| | | | | | H238, F272-H294, | |
| | | | | | F487-H509, Y244- | |
| | | | | | H266, C188-H210, | |
| | | | | | Y300-H322, H515- | |
| | | | | | H537, F355-H377 | |
| | | | | | C2H2-type zinc finger | BLIMPS_PRINTS |
| | | | | | signature PR00048: | |
| | | | | | P243-K256, L287- | |
| | | | | | G296 | |
| | | | | | Zinc finger, C2H2 type | BLIMPS_BLOCKS |
| | | | | | BL00028: C545-H561 | |
| | | | | | Protein Zinc finger | BLIMPS_PRODOM |
| | | | | | PD00066: H290-C302 | |
| | | | | | PROTEIN ZINC FINGER | BLAST_PRODOM |
| | | | | | METAL-BINDING DNA- | |
| | | | | | BINDING | |
| | | | | | PD170001: W111-G186 | |
| | | | | | PD167819: A390-F432 | |
| | | | | | PD017719: G184-G385 | |
| | | | | | PD000072: R214-C277, | |
| | | | | | R242-C305 | |
| | | | | | ZINC FINGER, C2H2 TYPE, | BLAST_DOMO |
| | | | | | DOMAIN | |
| | | | | | DM00002\|P08042\| | |
| | | | | | 272-312: Q263-Q304 | |
| | | | | | Aldehyde dehydrogenases | MOTIFS |
| | | | | | cysteine active site | |
| | | | | | A68-K79 | |
| | | | | | Zinc finger, C2H2 type, | MOTIFS |
| | | | | | domain C162-H182 | |
| | | | | | C188-H210 C190- | |
| | | | | | H210 C218-H238 | |
| | | | | | C246-H266 C274- | |
| | | | | | H294 C302-H322 | |
| | | | | | C357-H377 C433- | |
| | | | | | H453 C461-H481 | |
| | | | | | C489-H509 C517- | |
| | | | | | H537 C545-H565 | |
| 17 | 71259816CD1 | 281 | S73 S101 S131 | N237 | | |
| | | | S212 T122 T130 | | | |
| | | | T161 T221 | | | |
| 18 | 3354130CD1 | 518 | S103 S302 S317 | N196 | Signal_cleavage: | SPSCAN |
| | | | S346 S402 S421 | | M1-N14 | |
| | | | S471 S499 T24 T84 | | | |
| | | | T236 T275 T294 | | | |
| | | | T389 T400 T406 | | | |
| | | | T456 Y246 Y407 | | | |
| | | | | | Zinc finger C2H2 type: | HMMER_PFAM |
| | | | | | H351-H373, Y379- | |
| | | | | | H401 | |
| | | | | | SCAN domain: T24-V119 | HMMER_PFAM |
| | | | | | Zinc finger, C2H2 type: | HMMER_PFAM |
| | | | | | F463-H485, H323- | |
| | | | | | H345, H435-H457, | |
| | | | | | Y407-H429, Y491- | |
| | | | | | H513 | |
| | | | | | C2H2-type zinc finger | BLIMPS_PRINTS |
| | | | | | signature PR00048: | |
| | | | | | P350-S363, L422-G431 | |
| | | | | | Zinc finger, C2H2 type: | BLIMPS_BLOCKS |
| | | | | | C353-H369 | |
| | | | | | METAL-BINDING ZINC | BLAST_PRODOM |
| | | | | | FINGER PROTEIN DNA- | |
| | | | | | BINDING | |
| | | | | | PD004640: N14-E144 | |
| | | | | | PD017719: K314-H513, | |
| | | | | | G375-G516, G347- | |
| | | | | | H485, G319-F500 | |
| | | | | | PD000072: R377-C440, | |
| | | | | | Y407-C468, K433- | |
| | | | | | C496, K321-C384, | |
| | | | | | E349-C412 | |
| | | | | | P18; DM03974\| | BLAST_DOMO |
| | | | | | Q07231\|165-306: | |
| | | | | | L73-P153 | |
| | | | | | P18; FINGER; ZINC; | BLAST_DOMO |
| | | | | | DM03735\|P49910\| | |
| | | | | | 45-90: E27-L72 | |
| | | | | | ZINC FINGER, C2H2 TYPE, | BLAST_DOMO |
| | | | | | DOMAIN DM00002 | |
| | | | | | Q05481\|789-829: | |
| | | | | | Q342-C381 DM00002\| | |
| | | | | | P08042\|314-358: | |
| | | | | | C440-H485, C468- | |
| | | | | | H513, C356-H401 | |
| | | | | | Zinc finger, C2H2 type, | MOTIFS |
| | | | | | domain: C325-H345 | |
| | | | | | C353-H373 C381- | |
| | | | | | H401 C409-H429 | |
| | | | | | C437-H457 465- | |
| | | | | | H485 C493-H513 | |
| 19 | 1797985CD1 | 1033 | S88 S147 S253 | N221 N435 N436 | DnaJ domain: D91-D155 | HMMER_PFAM |
| | | | S415 S425 S437 | N655 | | |
| | | | S448 S503 S556 | | | |
| | | | S592 S618 S633 | | | |
| | | | S867 T13 T54 | | | |
| | | | T196 T347 T394 | | | |
| | | | T438 T731 Y180 | | | |
| | | | Y463 | | | |
| | | | | | Transmembrane domains: | TMAP |
| | | | | | S316-Y344 V549- | |
| | | | | | H574 N598-L622 | |
| | | | | | G766-W784; N-terminus | |
| | | | | | cytosolic | |
| | | | | | Leucine zipper pattern | MOTIFS |
| | | | | | L817-L838 | |
| 20 | 2870383CD1 | 486 | S19 S50 S64 S418 | N284 | Signal_cleavage: | SPSCAN |
| | | | S426 T127 T141 | | M1-G15 | |
| | | | T220 T393 T402 | | | |
| | | | Y466 | | | |
| | | | | | Signal Peptide: | HMMER |
| | | | | | M1-P17 | |
| | | | | | ATP/GTP-binding site | MOTIFS |
| | | | | | motif A (P-loop): | |
| | | | | | G447-T454 | |
| 21 | 1285088CD1 | 485 | S12 S86 S94 S95 | N70 N81 N143 | Signal_cleavage: | SPSCAN |
| | | | S96 S181 S235 | N330 N414 | M1-A39 | |
| | | | S244 S260 S283 | | | |
| | | | S320 S347 S464 | | | |
| | | | T196 T387 | | | |
| | | | | | ELM2 domain: | HMMER_PFAM |
| | | | | | G103-A167 | |
| | | | | | Myb-like DNA-binding | HMMER_PFAM |
| | | | | | domain: N383-R428, | |
| | | | | | P192-K237 | |
| | | | | | ATP/GTP-binding site | MOTIFS |
| | | | | | motif A (P-loop): | |
| | | | | | A206-T213 | |
| 22 | 1532441CD1 | 751 | S9 S72 S369 S394 | | B-box zinc finger: | HMMER_PFAM |
| | | | S507 S535 S567 | | R164-L205, A96- | |
| | | | S736 T117 T143 | | L149 | |
| | | | T253 T402 T418 | | | |
| | | | T426 T427 T434 | | | |
| | | | T442 T695 T729 | | | |
| | | | Y152 | | | |
| | | | | | Zinc finger C3HC4 type | HMMER_PFAM |
| | | | | | (RING finger): C21- | |
| | | | | | C59 | |
| | | | | | Transmembrane domains: | TMAP |
| | | | | | G542-L570; N-terminus | |
| | | | | | cytosolic | |
| | | | | | PROTEIN ZINC FINGER | BLAST_PRODOM |
| | | | | | NUCLEAR TRANSCRIPTION | |
| | | | | | INTERMEDIARY FACTOR | |
| | | | | | REGULATION REPRESSOR | |
| | | | | | DNA-BINDING FINGER | |
| | | | | | PD013917: R164-H355 | |
| | | | | | W04H10.3 PROTEIN | BLAST_PRODOM |
| | | | | | PD181144: L11-K87 | |
| | | | | | Eukaryotic putative | MOTIFS |
| | | | | | RNA-binding region RNP-1 | |
| | | | | | signature: K689-L696 | |
| | | | | | Zinc finger, C3HC4 type | MOTIFS |
| | | | | | (RING finger), sig- | |
| | | | | | nature: C36-L45 | |
| 23 | 3056408CD1 | 1786 | S168 S192 S255 | N231 N319 N336 | ARID DNA binding domain: | HMMER_PFAM |
| | | | S502 S581 S677 | N396 N452 N468 | L600-T709 | |
| | | | S705 S739 S772 | N469 N489 N840 | | |
| | | | S790 S802 S842 | N1031 N1209 | | |
| | | | S949 S992 S1054 | N1567 N1748 | | |
| | | | S1114 S1179 S1244 | | | |
| | | | S1260 S1265 S1289 | | | |
| | | | S1319 S1340 S1373 | | | |
| | | | S1419 S1437 S1449 | | | |
| | | | S1462 S1545 S1627 | | | |
| | | | S1643 S1698 T174 | | | |
| | | | T280 T589 T615 | | | |
| | | | T709 T1164T1292 | | | |
| | | | T1303 T1369 | | | |
| | | | T1413 Y917 Y929 | | | |
| | | | | | Transmembrane domains: | TMAP |
| | | | | | L1220-Y1238, | |
| | | | | | E1756-11776; | |
| | | | | | N-terminus non-cytosolic | |
| | | | | | PROTEIN BINDING NUCLEAR | BLAST_PRODOM |
| | | | | | DNA HOMOLOG TRANSCRIP- | |
| | | | | | TION DRIL1 RETINO- | |
| | | | | | BLASTOMA TRANSACTING | |
| | | | | | FACTOR PD004601: | |
| | | | | | E605-P699 | |
| | | | | | B120 | BLAST_PRODOM |
| | | | | | PD067746: E939-P1026 | |
| | | | | | PD123703: R875-Q937 | |

TABLE 4

| Polynucleotide | |
|---|---|
| SEQ ID NO:/ | |
| Incyte | |
| ID/Sequence | |
| Length | Sequence Fragments |
| 24/4936875CB1/ | 1-274, 1-279, 1-390, 1-798, 5-284, |
| 6790 | 5-532, 6-289, 13-377, 13-393, 17-593, |
| | 42-505, 60-688, 64-635, 134-663, 260-635, |
| | 296-677, 360-634, 423-982, 445-785, 552- |
| | 1076, 561-690, 621-950, 738-1389, 843-1406, |
| | 893-1146, 1044-6576, 1114-1414, 1134-1402, |
| | 1155-1436, 1155-1459, 1224-1708, 1228-1636, |
| | 1232-1848, 1270-1885, 1286-1534, 1446-1705, |
| | 1446-1721, 1648-2447, 1674-2421, 1695-2485, |
| | 1796-2574, 1825-2548, 1840-1868, 1840-1937, |
| | 1906-2140, 1927-2482, 1947-2553, 1948-2648, |
| | 2013-2602, 2058-2681, 2066-2600, 2166-2818, |
| | 2222-2780, 2347-3274, 2456-3109, 2536-2657, |
| | 2597-3250, 2598-3181, 2624-3296, 2666-3217, |
| | 2717-3271, 2742-3389, 2874-3412, 2874-3484, |
| | 2886-3724, 2925-3735, 2996-3571, 3001-3727, |
| | 3023-3590, 3047-3698, 3243-3973, 3248-3959, |
| | 3391-3971, 3562-4137, 3629-4355, 3654-4369, |
| | 3675-4466, 3677-4533, 3722-4665, 3734-4314, |
| | 3749-4377, 3766-4343, 3766-4464, 3772-4652, |
| | 3789-4678, 3824-4492, 3829-4466, 3835-4396, |
| | 3858-4601, 3872-4485, 3941-3965, 3944-4570, |
| | 3956-4698, 3959-4518, 3968-4518, 3969-4619, |
| | 3993-4794, 4019-4597, 4022-4712, 4022-4727, |
| | 4059-4640, 4059-4787, 4066-4710, 4067-4685, |
| | 4082-4641, 4083-4865, 4103-4967, 4108-4631, |
| | 4113-4775, 4125-4687, 4134-4803, |
| | 4165-5002, 4186-4796, 4193-4942, 4197-5001, |
| | 4215-4838, 4219-5067, 4295-5036, 4296-4405, |
| | 4296-4540, 4298-4389, 4305-5129, 4308-5011, |
| | 4327-4980, 4331-5047, 4357-4385, 4367-5059, |
| | 4389-5237, 4393-4984, 4394-4928, 4394-5152, |
| | 4409-4950, 4438-5126, 4473-5085, 4474-5033, |
| | 4496-5048, 4553-5236, 4572-5128, 4578-5129, |
| | 4619-5390, 4636-5388, 4653-5213, 4657-5387, |
| | 4674-5408, 4686-5305, 4703-5387, 4708-5333, |
| | 4718-5399, 4743-5567, 4749-5460, 4784-5467, |
| | 4823-5502, 4845-5399, 4862-5541, 4864-5492, |
| | 4873-5469, 4896-5541, 4936-5560, 4939-5608, |
| | 4955-5642, 5002-5573, 5013-5667, 5047-5618, |
| | 5073-5608, 5081-5713, 5083-5698, 5097-5668, |
| | 5099-5743, 5113-5752, 5133-5896, 5142-5722, |
| | 5165-5698, 5189-5737, 5191-5774, 5199-5725, |
| | 5201-5756, 5203-5845, 5210-5746, 5223-5881, |
| | 5228-5892, 5246-5936, 5256-5896, 5272-5893, |
| | 5305-6012, 5307-5894, 5347-5949, 5362-6049, |
| | 5371-5986, 5384-6038, 5392-6061, 5408-5944, |
| | 5428-6209, 5455-6125, 5457-6003, 5470-6219, |
| | 5507-6151, 5554-6185, 5556-6098, 5571-6163, |
| | 5572-6105, 5572-6115, 5572-6128, 5572-6136, |
| | 5572-6158, 5572-6165, 5572-6176, 5578-6238, |
| | 5579-6247, 5583-6158, 5594-6248, 5599-6208, |
| | 5604-6204, 5610-6227, 5615-6239, 5623-6298, |
| | 5637-6220, |
| | 5655-6335, 5662-6271, 5670-6200, 5858-6710, |
| | 5907-6537, 5957-6577, 6010-6777, 6011-6726, |
| | 6014-6783, 6016-6720, 6030-6591, 6042-6788, |
| | 6056-6776, 6062-6612, 6071-6771, 6083-6717, |
| | 6090-6759, 6109-6762, 6125-6782, 6126-6778, |
| | 6135-6766, 6136-6708, 6136-6753, 6137-6755, |
| | 6140-6790, 6151-6790, 6157-6762, 6158-6765, |
| | 6159-6782, 6159-6787, 6162-6782, 6169-6751, |
| | 6177-6762, 6178-6695, 6188-6746, 6195-6773, |
| | 6199-6762, 6200-6733, 6206-6717, 6224-6773, |
| | 6227-6749, 6233-6748, 6235-6666, 6235-6765, |
| | 6239-6769, 6245-6776, 6255-6731, 6261-6755, |
| | 6261-6772, 6266-6716, 6273-6782, 6276-6790, |
| | 6289-6764, 6293-6776, 6296-6770, 6301-6777, |
| | 6303-6790, 6304-6773, 6307-6770, 6311-6790, |
| | 6315-6771, 6316-6773, 6316-6790, 6318-6551, |
| | 6321-6765, 6323-6769, 6323-6770, 6325-6770, |
| | 6329-6770, 6332-6687, 6341-6773, 6342-6770, |
| | 6344-6771, 6344-6773, 6346-6775, 6347-6739, |
| | 6351-6770, 6351-6772, 6352-6769, 6354-6773, |
| | 6356-6771, 6358-6770, 6360-6771, 6360-6773, |
| | 6361-6788, 6362-6774, 6363-6773, 6364-6773, |
| | 6365-6770, 6367-6757, 6367-6773, 6367-6790, |
| | 6370-6770, 6374-6773, 6379-6773, 6380-6790, |
| | 6381-6773, 6384-6770, 6388-6770, 6407-6768, |
| | 6411-6770, 6411-6772, 6411-6773, 6412-6770, |
| | 6412-6773, 6416-6770, 6417-6685, 6418-6712, |
| | 6425-6679, 6426-6785, 6433-6740, 6434-6768, |
| | 6436-6716, 6437-6712, 6439-6789, 6451-6770, |
| | 6457-6773, 6459-6770, 6460-6732, 6464-6784, |
| | 6465-6714, 6465-6770, 6469-6696, 6480-6710, |
| | 6482-6698, 6484-6773, 6490-6767, 6494-6768, |
| | 6495-6734, 6495-6772, 6496-6778, 6497-6766, |
| | 6509-6776, 6510-6773, 6511-6781, 6513-6769, |
| | 6513-6773, 6516-6691, 6526-6772, 6534-6785, |
| | 6540-6773, 6561-6770, 6566-6694, 6572-6770, |
| | 6576-6717, 6593-6790, 6599-6698, 6611-6732, |
| | 6739-6773 |
| 25/264408CB1/ | 1-684, 80-785, 301-622, 336-602, 345-849, |
| 4859 | 367-1043, 405-1074, 904-1226, 904-1273, |
| | 905-1273, 906-1193, 1188-1774, 1266-1360, |
| | 1340-1844, 1340-1955, 1432-2181, 1520-1616, |
| | 1661-1888, 1672-1922, 1761-1919, 1761-2127, |
| | 1761-2147, 1761-2162, 1761-2166, 1761-2194, |
| | 1761-2200, 1761-2204, 1761-2207, 1761-2243, |
| | 1761-2247, 1761-2319, 1761-2412, 1790-2319, |
| | 1922-2688, 1961-2380, 1963-2161, 2024-2551, |
| | 2046-2521, 2142-2656, 2144-2423, 2150-2287, |
| | 2194-2454, 2201-2948, 2223-2719, 2226-2415, |
| | 2226-2688, 2251-2857, 2254-2562, 2296-2617, |
| | 2309-2932, 2310-2932, 2344-2932, 2364-2892, |
| | 2366-2932, 2367-2932, 2376-2932, 2380-2932, |
| | 2381-2932, 2386-2932, 2387-2932, 2393-2932, |
| | 2409-2932, 2412-3085, 2413-2932, 2419-2932, |
| | 2419-2998, 2426-2932, 2426-3106, 2427-2932, |
| | 2431-2932, 2432-2963, 2434-2932, 2442-2932, |
| | 2449-2932, 2475-2932, 2477-2932, 2484-2932, |
| | 2486-3028, 2486-3035, 2487-2932, 2492-2932, |
| | 2510-2932, 2516-2932, 2517-2932, 2524-2930, |
| | 2551-2932, 2583-2932, 2638-2932, 2639-3202, |
| | 2718-3276, 2720-2973, 2920-3177, 2920-3508, |
| | 2961-3214, 2961-3471, 3007-3528, 3009-3271, |
| | 3010-3274, 3029-3287, 3103-3458, 3119-3635, |
| | 3159-3406, 3159-3751, 3281-3998, 3327-3630, |
| | 3408-3656, 3409-3614, |
| | 3451-3596, 3499-3743, 3528-3817, 3567-3823, |
| | 3588-3727, 3611-4212, 3647-4142, 3648-3952, |
| | 3653-4301, 3710-3981, 3726-4209, 3743-3973, |
| | 3789-3900, 3798-4192, 3814-4008, 3814-4231, |
| | 3880-4030, 3880-4236, 3896-4142, 3907-4570, |
| | 3908-4510, 3931-4111, 3935-4229, 3948-4603, |
| | 3982-4143, 3982-4207, 3990-4300, 4004-4199, |
| | 4011-4694, 4026-4612, 4069-4309, 4107-4386, |
| | 4126-4418, 4130-4372, 4165-4831, 4170-4859, |
| | 4173-4611, 4175-4420, 4201-4646, 4227-4451, |
| | 4233-4610, 4237-4859 |
| 26/2181434CB1/ | 1-913, 283-1089, 497-739, 497-908, 497-1042, |
| 3336 | 497-1048, 497-1054, 497-1075, 595-916, 600-1374, |
| | 691-917, 710-1303, 715-915, 715-1020, 912-1284, |
| | 916-1162, 923-1539, 977-1601, 993-1284, 1001-1284, |
| | 1021-1162, 1021-1284, 1063-1284, 1132-1284, |
| | 1163-1432, 1191-1442, 1191-1747, 1191-1762, |
| | 1274-1756, 1281-1355, 1281-1390, 1281-1412, |
| | 1281-1545, 1284-1614, 1285-1432, 1285-1575, |
| | 1353-1728, 1368-1707, 1378-1887, 1400-1972, |
| | 1405-1653, 1428-1808, 1429-1988, 1433-1575, |
| | 1433-1727, 1450-2048, 1452-2016, 1465-1739, |
| | 1478-2052, 1491-1857, 1510-2090, 1511-2042, |
| | 1517-2052, 1521-2065, 1532-1804, 1538-2010, |
| | 1544-2194, 1552-1872, 1561-2225, 1565-2201, |
| | 1576-1727, 1576-1790, 1605-2187, 1609-2126, |
| | 1612-2182, 1618-2209, 1619-2334, 1624-2368, |
| | 1625-1791, 1684-2222, 1689-2175, 1691-2201, |
| | 1709-1956, 1718-1977, 1727-1920, 1727-2028, |
| | 1727-2044, 1728-1917, 1731-2001, 1731-2212, |
| | 1735-2005, 1762-2008, 1767-2392, 1770-2017, |
| | 1783-2074, 1788-1935, 1791-1917, 1871-2107, |
| | 1895-2153, 1913-2510, 1927-2191, 1928-2237, |
| | 1933-2564, 1952-2617, 1961-2621, 1972-2533, |
| | 1983-2593, 1996-2388, 1997-2558, 2015-2521, |
| | 2015-2530, 2021-2630, 2026-2627, 2070-2466, |
| | 2089-2361, 2100-2605, 2110-2564, 2121-2735, |
| | 2123-2564, 2124-2681, 2129-2380, 2129-2549, |
| | 2129-2641, 2154-2466, 2167-2453, 2178-2419, |
| | 2178-2431, 2182-2453, 2191-2462, 2191-2809, |
| | 2191-2878, 2211-2466, 2211-2467, 2235-2702, |
| | 2249-2410, 2250-2821, 2254-2517, 2279-2470, |
| | 2279-2844, 2317-2609, 2317-3070, 2319-2855, |
| | 2321-2711, 2335-2594, 2335-2911, 2337-2588, |
| | 2353-2902, 2366-2564, 2376-2863, 2394-2854, |
| | 2396-2915, 2405-2924, 2406-2678, 2417-2909, |
| | 2434-3279, 2452-2908, 2465-2724, 2487-2740, |
| | 2497-3053, 2534-2821, 2538-2799, 2549-2784, |
| | 2558-3285, 2581-3278, 2604-3014, 2604-3244, |
| | 2611-3260, 2626-2862, 2633-3128, 2644-3331, |
| | 2655-3288, 2659-3331, 2660-3259, 2677-3260, |
| | 2691-2943, 2704-2909, 2711-2977, 2711-3085, |
| | 2711-3234, 2711-3313, 2721-3319, 2721-3331, |
| | 2723-3315, 2731-2915, 2731-3015, 2733-2978, |
| | 2733-3319, 2741-3331, 2780-3047, 2784-3298, |
| | 2785-3014, 2785-3025, 2803-3323, 2812-3336, |
| | 2831-3289, 2842-3316, 2847-3108, 2876-3331, |
| | 2878-3320, 2880-3318, 2895-3331, 2915-3270, |
| | 2916-3114, 2936-3331, 2982-3290, 2993-3270, |
| | 2993-3287, 2993-3289, 3010-3176, 3010-3331, |
| | 3062-3331, 3065-3243, 3079-3314, 3079-3317, |
| | 3079-3331, 3094-3319 |
| 27/1367252CB1/ | 1-514, 75-588, 112-587, 356-973, 489-1156, |
| 2918 | 525-1173, 625-916, 826-1023, 826-1067, |
| | 826-1397, 826-1426, 866-1512, 896-1315, |
| | 1126-1340, 1126-1697, 1212-1522, 1214-1663, |
| | 1246-1541, 1399-2033, 1400-1666, 1400-1832, |
| | 1442-1713, 1442-1948, 1458-1755, 1483-1778, |
| | 1484-1812, 1562-2121, 1600-2128, 1619-1910, |
| | 1626-1901, 1718-2251, 1738-1982, 1748-2071, |
| | 1761-1992, 1761-1994, 1761-2296, 1789-2312, |
| | 1850-2094, 1860-2099, 1868-2490, 1875-2131, |
| | 1899-2575, 1956-2253, 1956-2258, 1956-2361, |
| | 1972-2571, 2047-2181, 2057-2400, 2090-2508, |
| | 2097-2515, 2112-2501, 2123-2380, 2131-2581, |
| | 2131-2589, 2174-2586, 2178-2501, 2193-2668, |
| | 2228-2499, 2272-2510, 2279-2493, 2335-2550, |
| | 2336-2617, 2350-2804, 2414-2715, 2429-2589, |
| | 2527-2813, 2527-2814, 2527-2823, 2670-2918 |
| 28/5633694CB1/ | 1-260, 1-319, 1-643, 1-645, |
| 1610 | 1-1610, 7-172, 7-516, 13-575, |
| | 14-284, 14-520, 16-531, 17-680, |
| | 19-318, 24-280, 24-305, 24-306, |
| | 24-325, 24-438, 28-296, 33-213, |
| | 49-289, 70-381, 76-306, 86-358, |
| | 187-755, 191-674, 245-768, 251-538, |
| | 296-886, 297-717, 315-872, 315-881, |
| | 316-881, 326-883, 389-858, 392-858, |
| | 395-858, 397-858, 399-889, 402-871, |
| | 407-880, 408-858, 412-858, 412-871, |
| | 413-881, 415-858, 419-858, 420-858, |
| | 424-705, 450-882, 455-882, 457-882, |
| | 460-870, 464-845, 485-927, 492-858, |
| | 494-856, 499-758, 499-838, 499-858, |
| | 500-800, 501-801, 501-840, 501-851, |
| | 501-858, 501-871, 501-888, 506-881, |
| | 507-800, 509-858, 510-881, 538-858, |
| | 538-871, 548-868, 549-858, 552-881, |
| | 553-876, 553-881, 568-859, 568-871, |
| | 581-858, 586-1274, 600-858, 613-858, |
| | 616-889, 649-860, 667-858, 679-871, |
| | 690-858, 882-1226, 882-1260, 882-1283, |
| | 882-1313, 882-1323, 882-1359, 882-1383, |
| | 888-1441, 890-1411, 891-1447, 897-1126, |
| | 898-1394, 900-1357, 907-1181, 910-1121, |
| | 910-1175, 935-1174, 936-1242, 947-1247, |
| | 956-1276, 956-1349, 969-1299, 984-1350, |
| | 984-1396, 1003-1259, 1005-1604, 1031-1319, |
| | 1053-1527, 1058-1290, 1107-1292, 1119-1294, |
| | 1175-1481, 1201-1409, 1332-1610, 1335-1604, |
| | 1378-1574, 1459-1587 |
| 29/7985981CB1/ | 1-615, 1-935, 7-676, 147-653, 147-796 |
| 935 | |
| 30/4706628CB1/ | 1-480, 87-588, 271-886, 425-1001, |
| 3609 | 557-1102, 814-1042, 814-1466, 868-1418, |
| | 868-1448, 1027-1447, 1093-1604, 1095-1415, |
| | 1097-1324, 1153-1418, 1172-1444, 1249-1808, |
| | 1342-1589, 1342-1594, 1342-1685, 1374-1944, |
| | 1474-1704, 1474-1705, 1488-1761, 1558-2025, |
| | 1564-1813, 1564-1837, 1564-2052, 1590-2203, |
| | 1749-2020, 1817-2060, 1817-2307, 1967-2243, |
| | 1981-2266, 2018-2233, 2018-2597, 2079-2323, |
| | 2079-2684, 2139-2744, 2160-2411, 2161-2431, |
| | 2208-2512, 2228-2584, 2232-2513, 2257-2524, |
| | 2266-2441, 2266-2450, 2266-2810, 2290-2572, |
| | 2290-2767, 2318-2453, 2327-2645, 2368-2647, |
| | 2385-2695, 2409-2676, 2432-2663, 2454-2740, |
| | 2465-2812, 2484-2754, 2487-2755, 2545-2799, |
| | 2581-2819, 2581-2841, 2584-2828, 2585-2833, |
| | 2612-3210, 2613-2893, 2620-2901, 2623-3244, |
| | 2637-3115, 2649-2885, 2649-3195, 2663-2952, |
| | 2668-3227, 2668-3372, 2672-2818, 2675-2956, |
| | 2692-2956, 2750-2983, 2769-3370, 2777-2980, |
| | 2777-3000, 2781-2984, 2785-3042, 2805-3082, |
| | 2814-3341, 2815-3368, 2836-3214, 2850-3107, |
| | 2850-3124, 2850-3131, 2864-3108, 2866-3078, |
| | 2872-3205, 2877-3604, 2886-3084, 2886-3140, |
| | 2908-3589, 2913-3512, 2916-3354, 2940-3210, |
| | 2948-3609, 2951-3558, 2955-3198, 2956-3219, |
| | 2956-3466, 2957-3599, |
| | 2994-3257, 2996-3232, 2996-3338, 2999-3022, |
| | 3003-3593, 3009-3590, 3010-3528, 3015-3464, |
| | 3021-3593, 3022-3304, 3024-3342, 3025-3600, |
| | 3026-3285, 3037-3303, 3039-3303, 3045-3609, |
| | 3048-3283, 3054-3591, 3057-3588, 3068-3593, |
| | 3070-3609, 3088-3279, 3095-3341, 3103-3565, |
| | 3113-3592, 3131-3600, 3139-3401, 3143-3433, |
| | 3145-3443, 3146-3407, 3147-3403, 3154-3608, |
| | 3156-3449, 3156-3605, 3162-3595, 3163-3432, |
| | 3163-3600, 3164-3600, 3173-3600, 3180-3600, |
| | 3194-3606, 3198-3600, 3201-3605, 3202-3600, |
| | 3202-3606, 3212-3473, 3215-3465, 3223-3606, |
| | 3225-3584, 3229-3504, 3235-3609, 3239-3600, |
| | 3244-3606, 3244-3607, 3284-3558, 3300-3600, |
| | 3336-3579, 3336-3609, 3347-3609, 3370-3609, |
| | 3387-3606, 3388-3609, 3406-3599, 3418-3606, |
| | 3426-3600 |
| 31/5790110CB1/ | 1-459, 280-433, 295-915, 301-1226, |
| 4136 | 316-616, 342-914, 360-913, 362-990, |
| | 422-1274, 540-907, 540-925, 735-925, |
| | 916-1310, 916-1458, 916-1588, 916-1627, |
| | 917-1466, 917-1548, 918-1383, 918-1414, |
| | 918-4136, 920-1593, 921-1400, 923-1598, |
| | 924-1482, 925-1229, 945-1337, 956-1398, |
| | 969-1422, 983-1126, 986-1366, 997-1118, |
| | 997-1130, 997-1207, 997-1231, 997-1243, |
| | 997-1247, 997-1268, 997-1271, 997-1274, |
| | 997-1303, 997-1310, 997-1313, 997-1314, |
| | 997-1348, 997-1349, 997-1353, 997-1363, |
| | 997-1383, 997-1385, 997-1386, 997-1387, |
| | 997-1388, 997-1389, 997-1391, 997-1392, |
| | 997-1393, 997-1395, 997-1397, 997-1398, |
| | 997-1399, 997-1400, 997-1401, 997-1402, |
| | 997-1404, 997-1406, 997-1407, 997-1409, |
| | 997-1411, 997-1412, 997-1413, 997-1414, |
| | 997-1416, 997-1421, 997-1422, 997-1425, |
| | 997-1426, 997-1427, 997-1428, 997-1433, |
| | 997-1434, 999-1400, 1007-1378, 1041-1158, |
| | 1110-1310, 1117-1515, 1132-1695, 1136-1695, |
| | 1188-1444, 1195-1512, 1201-1539, 1277-1512, |
| | 1290-1983, 1303-1972, 1317-1336, 1320-2137, |
| | 1322-1664, 1322-1694, 1331-1925, 1344-1745, |
| | 1346-1592, 1356-1873, 1357-2224, 1365-1826, |
| | 1365-1828, 1366-1836, 1370-1983, 1403-2079, |
| | 1410-2101, 1422-1706, 1422-2117, 1438-1684, |
| | 1461-1932, 1463-1969, |
| | 1470-1896, 1470-1913, 1470-1974, 1472-1953, |
| | 1473-1834, 1482-1552, 1482-1955, 1483-1740, |
| | 1495-2015, 1497-2137, 1501-2030, 1508-1830, |
| | 1516-1816, 1520-2054, 1522-2056, 1558-1809, |
| | 1587-2111, 1600-2165, 1619-1930, 1637-2332, |
| | 1657-2137, 1663-1939, 1664-1913, 1664-2214, |
| | 1665-2214, 1674-1885, 1719-1770, 1722-1770, |
| | 1729-2248, 1729-2277, 1730-1967, 1745-1833, |
| | 1747-1972, 1755-2220, 1764-2353, 1772-2061, |
| | 1773-2056, 1773-2305, 1783-2250, 1783-2282, |
| | 1784-2269, 1784-2318, 1800-2314, 1824-2105, |
| | 1833-2338, 1834-2339, 1841-2312, 1854-2083, |
| | 1854-2113, 1854-2128, 1879-2379, 1883-2364, |
| | 1889-2143, 1889-2162, 1899-2537, 1912-2170, |
| | 1916-2267, 1919-2549, 1933-2211, 1944-2227, |
| | 1944-2439, 1949-2448, 1959-2522, 1967-2469, |
| | 1972-2586, 1977-2195, 1977-2213, 1977-2236, |
| | 1982-2241, 1982-2300, 1986-2254, 1992-2476, |
| | 2021-2577, 2023-2527, 2023-2826, 2044-2303, |
| | 2046-2721, 2052-2269, 2052-2650, 2054-2364, |
| | 2056-2222, 2056-2371, 2058-2316, 2073-2324, |
| | 2073-2342, 2082-2453, 2084-2934, 2091-2269, |
| | 2106-2373, 2106-2811, 2117-2366, 2118-2399, |
| | 2119-2617, 2129-2515, 2131-2405, 2135-2316, |
| | 2135-2716, 2135-2740, 2139-2642, 2140-2418, |
| | 2142-2508, 2143-2375, 2145-2403, 2150-2694, |
| | 2150-2745, 2157-2416, 2187-2925, 2187-2949, |
| | 2191-2426, 2191-2429, 2199-2913, 2200-2648, |
| | 2206-2441, 2208-2471, 2213-2285, 2213-2454, |
| | 2217-2441, 2224-2484, 2227-2481, 2229-2505, |
| | 2230-2949, 2235-2874, 2240-2419, 2243-2519, |
| | 2243-2524, 2245-2707, 2249-2663, 2250-2433, |
| | 2254-2512, 2254-2935, 2255-2790, 2259-2536, |
| | 2261-2729, 2263-2712, 2264-2675, 2269-2528, |
| | 2271-2501, 2279-2532, 2279-2770, 2291-2947, |
| | 2302-2757, 2303-2576, 2303-2933, 2306-2938, |
| | 2311-2547, 2319-2891, 2322-2650, 2326-2892, |
| | 2328-2459, 2342-2604, 2356-2846, 2357-2827, |
| | 2359-2809, 2366-2884, 2381-2917, 2382-2648, |
| | 2382-2683, 2382-2696, 2384-2441, 2387-2657, |
| | 2398-2896, 2403-2662, 2405-2949, 2413-2645, |
| | 2414-2943, 2445-2752, 2448-2852, 2456-2765, |
| | 2468-2948, 2469-2948, 2471-2949, 2472-2553, |
| | 2477-2919, 2480-2709, 2481-2847, 2481-2941, |
| | 2483-2955, 2485-2944, 2490-2950, 2494-2751, |
| | 2494-2948, 2494-2949, 2498-2943, 2502-2948, |
| | 2504-2949, 2508-2808, 2510-2943, 2513-2949, |
| | 2515-2948, 2515-2949, 2516-2949, 2517-2761, |
| | 2517-2949, 2518-2948, 2518-2949, 2520-2948, |
| | 2522-2948, 2531-2901, 2533-2949, 2541-2948, |
| | 2541-2949, 2543-2955, 2544-2949, 2547-2949, |
| | 2549-2948, 2550-2860, 2550-2949, 2551-2949, |
| | 2557-2852, 2557-2948, 2558-2943, 2578-2949, |
| | 2588-2899, 2608-2845, 2609-2949, 2611-3302, |
| | 2614-2948, 2615-2949, 2617-2916, 2617-2950, |
| | 2619-2947, 2622-2901, 2632-2893, 2634-2868, |
| | 2639-2948, 2639-2949, 2649-2943, 2655-2906, |
| | 2655-2913, 2661-2928, 2662-2949, 2675-2923, |
| | 2713-2948, 2719-2916, 2719-2931, 2730-2949, |
| | 2734-2948, 2753-2949, 3047-3710, 3099-3726, |
| | 3210-3876, 3363-4027, 3566-4136, 3654-4116, |
| | 3655-4111 |
| 32/2948827CB1/ | 1-1747, 14-760, 232-536, 456-536, |
| 2850 | 541-1060, 541-1200, 574-845, 683-1048, |
| | 684-968, 742-1449, 1185-2750, 1185-2850, |
| | 1296-1814, 1306-1586, 1348-1602, 1358-1609, |
| | 1363-1593, 1383-2094, 1483-1752, 1577-2321, |
| | 1616-1877, 1649-1892, 1649-2222, 1729-2341, |
| | 1736-2009, 1753-2337, 1794-1986, 1824-2265, |
| | 1883-2276, 1925-2366, 1929-2273, 1943-2355, |
| | 1952-2355, 1953-2355, 1968-2357, 1979-2358, |
| | 2007-2355, 2010-2267, 2043-2355, 2096-2325, |
| | 2096-2359, 2138-2356, 2141-2594 |
| 33/1398040CB1/ | 1-499 |
| 499 | |
| 34/7716061CB1/ | 1-712, 398-484, 622-706 |
| 712 | |
| 35/6113748CB1/ | 1-541, 3-530, 11-562, 16-310, |
| 1793 | 16-747, 450-1043, 471-769, 502-781, |
| | 567-870, 629-969, 997-1200, 997-1570, |
| | 1049-1466, 1077-1260, 1077-1368, 1260-1793 |
| 36/7474037CB1/ | 1-249, 1-297, 2-207, 2-285, |
| 858 | 2-344, 3-559, 5-213, 5-244, |
| | 5-292, 5-485, 5-520, 5-561, |
| | 5-581, 9-664, 10-261, 13-253, |
| | 19-194, 19-196, 19-261, 19-266, |
| | 19-271, 19-275, 19-290, 19-302, |
| | 20-231, 20-257, 20-290, 20-312, |
| | 20-337, 20-782, 21-288, 23-272, |
| | 23-287, 23-306, 23-308, 23-311, |
| | 23-717, 24-226, 24-265, 24-272, |
| | 25-269, 25-309, 25-314, 25-533, |
| | 27-300, 27-787, 28-271, 28-282, |
| | 29-247, 29-331, 29-363, 30-242, |
| | 30-278, 30-289, 30-473, 30-794, |
| | 31-330, 32-288, 32-326, 34-282, |
| | 34-285, 34-293, 34-303, 34-319, |
| | 34-326, 34-357, 34-714, 35-188, |
| | 35-235, 35-662, 36-284, 36-325, |
| | 39-651, 39-818, 43-294, 44-292, |
| | 44-313, 44-314, 49-325, 49-329, |
| | 50-379, 52-345, 55-362, 55-365, |
| | 66-210, 70-342, 73-400, 79-452, |
| | 81-306, 87-322, 105-361, 108-773, |
| | 109-336, 122-514, 125-304, 150-450, |
| | 162-458, 177-403, 195-797, 197-821, |
| | 206-416, 207-462, 208-450, 212-650, |
| | 246-494, 246-759, 246-825, 252-412, |
| | 253-484, 257-499, 266-853, 267-395, |
| | 276-536, 286-775, 311-499, 313-674, |
| | 337-597, 360-858, 370-660, 379-786, |
| | 379-792, 381-838, 384-838, 385-858, |
| | 387-857, 392-645, 392-669, 413-858, |
| | 445-716, 470-711, 483-640, 486-617, |
| | 489-727, 493-858, 500-790, 516-699, |
| | 516-758, 554-827, 569-780, 573-830, |
| | 578-834, 613-857, 615-858 |
| 37/2955646CB1/ | 1-258, 1-509, 1-519, 1-584, |
| 2387 | 283-784, 283-793, 429-562, 441-661, |
| | 470-661, 505-720, 609-1156, 628-1286, |
| | 656-880, 666-962, 716-1200, 732-1007, |
| | 740-1343, 741-799, 785-1450, 946-1232, |
| | 970-1559, 1011-1610, 1087-1420, 1088-1743, |
| | 1121-1686, 1162-1626, 1162-1628, 1244-1788, |
| | 1250-1899, 1256-1958, 1337-1959, 1342-1699, |
| | 1360-1938, 1468-1932, 1480-2063, 1511-2210, |
| | 1592-2079, 1592-2082, 1647-2082, 1685-1901, |
| | 1688-2249, 1823-2387 |
| 38/1573006CB1/ | 1-537, 1-673, 150-405, 150-558, |
| 2091 | 150-734, 150-764, 161-568, 161-671, |
| | 161-677, 161-717, 161-752, 161-770, |
| | 161-779, 161-792, 163-416, 164-377, |
| | 164-633, 186-863, 197-389, 197-714, |
| | 226-812, 226-877, 244-951, 304-987, |
| | 331-712, 332-831, 341-1008, 384-928, |
| | 420-1071, 436-1108, 506-1107, 542-1045, |
| | 551-920, 569-1185, 646-1093, 652-1265, |
| | 658-1284, 690-1304, 729-1198, 729-1382, |
| | 760-872, 760-884, 763-1294, 777-884, |
| | 790-920, 806-1093, 820-1093, 823-960, |
| | 823-978, 823-1007, 830-1124, 843-884, |
| | 845-883, 845-1007, 853-1215, 855-1007, |
| | 864-1007, 887-1307, 907-967, 907-1062, |
| | 907-1091, 918-1278, 928-1040, 928-1052, |
| | 929-1091, 934-1521, 938-1359, 939-1091, |
| | 945-1052, 948-1429, 991-1091, 999-1052, |
| | 999-1091, 1002-1091, 1011-1052, 1018-1091, |
| | 1023-1091, 1032-1557, 1054-1612, 1070-1680, |
| | 1120-1624, 1124-1624, 1136-1398, 1137-1669, |
| | 1220-1488, 1220-1493, 1220-1803, 1229-1875, |
| | 1298-1858, 1317-1902, 1321-1902, 1358-1926, |
| | 1387-1910, 1405-1921, 1413-2071, 1437-2091, |
| | 1468-2079, 1522-2052, 1522-2074, 1559-2091, |
| | 1588-2091, 1662-1910 |
| 39/1336756CB1/ | 1-758, 110-637, 210-645, 211-621, |
| 2385 | 214-645, 263-645, 334-1845, 374-990, |
| | 561-1165, 625-1028, 626-1036, 638-1038, |
| | 646-862, 691-978, 693-808, 694-744, |
| | 694-818, 694-829, 695-829, 723-1392, |
| | 766-1073, 778-913, 810-913, 839-1090, |
| | 861-976, 881-1150, 897-1082, 912-1401, |
| | 912-1531, 912-1534, 933-1082, 1025-1599, |
| | 1029-1249, 1029-1358, 1159-1542, 1196-1524, |
| | 1534-1726, 1534-1830, 1534-1966, 1534-1977, |
| | 1534-1985, 1534-1986, 1534-1987, 1534-2009, |
| | 1534-2012, 1534-2017, 1534-2037, 1534-2038, |
| | 1534-2045, 1535-1595, 1535-1670, 1535-1711, |
| | 1535-1780, 1535-1782, 1535-1797, 1535-1805, |
| | 1535-1901, 1535-1918, 1535-1929, 1535-1930, |
| | 1535-1935, 1535-1951, 1535-1968, 1535-1971, |
| | 1535-1980, 1535-1982, 1535-2005, 1535-2010, |
| | 1535-2011, 1535-2015, 1535-2019, 1535-2020, |
| | 1535-2021, 1535-2022, 1535-2023, 1535-2024, |
| | 1535-2025, 1535-2026, 1535-2027, 1535-2028, |
| | 1535-2030, 1535-2032, 1535-2033, 1535-2034, |
| | 1536-1931, 1536-2024, 1537-1845, 1537-2041, |
| | 1537-2050, 1538-1885, 1539-1651, 1543-1986, |
| | 1543-2124, 1545-1982, 1546-1867, 1553-1679, |
| | 1553-1802, 1553-1884, 1553-1920, 1553-1963, |
| | 1553-1972, 1553-1976, 1553-2006, 1553-2010, |
| | 1553-2013, 1553-2019, 1553-2020, 1553-2029, |
| | 1554-2013, 1571-1648, 1572-1966, 1578-1808, |
| | 1580-2038, 1583-1964, 1584-1735, 1585-1959, |
| | 1592-2012, 1619-1846, 1619-2175, 1628-2195, |
| | 1643-2131, 1661-1903, 1666-2193, 1699-2040, |
| | 1706-2197, 1741-2181, 1760-2191, 1771-2037, |
| | 1785-2196, 1789-2191, 1812-2197, 1839-2196, |
| | 1844-2189, 1846-2385, 1847-2025, 1847-2187, |
| | 1847-2197, 1892-2145, 1947-2197, 1979-2196, |
| | 1980-2197 |
| 40/71259816CB1/ | 1-501, 1-516, 1-1289, 2-596 |
| 1289 | 4-305, 4-386, 37-226, 142-713, 159-713, |
| | 206-713, 278-750, 365-713, 382-776, |
| | 415-713, 416-713, 484-711, 513-713, |
| | 527-713, 714-789, 714-846, 714-884, |
| | 714-904, 714-935, 714-1041, 714-1140, |
| | 773-928, 965-1289, 1025-1289 |
| 41/3354130CB1/ | 1-561, 1-563, 6-289, 6-516, 6-672, |
| 2628 | 7-580, 11-555, 27-317, 27-327, 27-546, |
| | 27-700, 224-773, 284-872, 323-777, |
| | 364-774, 376-779, 491-1038, 566-1022, |
| | 744-1303, 852-1024, 866-1485, 867-1378, |
| | 867-1401, 873-1359, 910-1127, 928-1178, |
| | 953-1524, 992-1501, 1009-1524, 1065-1620, |
| | 1107-1575, 1108-1186, 1147-1722, 1174-1395, |
| | 1186-1900, 1251-1903, 1276-1354, 1298-1358, |
| | 1434-1866, 1449-1680, 1458-2135, 1466-1526, |
| | 1481-1774, 1493-1720, 1494-2089, 1530-1793, |
| | 1569-1793, 1619-2221, 1779-2327, 1979-2405, |
| | 1979-2628, 1980-2267, 2030-2281 |
| 42/1797985CB1/ | 1-1830, 201-1086, 201-4010, 413-1212, |
| 4077 | 450-1099, 588-1162, 622-902, 675-1216, |
| | 687-898, 745-1002, 889-1182, 998-1457, |
| | 1043-1570, 1101-1551, 1124-1350, 1261-1556, |
| | 1353-1629, 1370-1829, 1486-2142, 1514-1706, |
| | 1515-2048, 1530-1797, 1607-2284, 1614- |
| | 2262, 1629-1995, 1649-2241, 1680-1928, |
| | 1803-2046, 1807-2067, 1846-2107, 1872-2498, |
| | 1887-2160, 1900-2143, 1919-4077, 1933-2561, |
| | 1939-2286, 1977-2205, 1988-2121, 2024- |
| | 2369, 2027-2334, 2066-2575, 2087-2323, |
| | 2100-2323, 2196-2428, 2196-2639, 2196-2653, |
| | 2196-2678, 2196-2681, 2196-2691, 2196-2764, |
| | 2196-2807, 2245-2813, 2269-2539, 2275-2524, |
| | 2276-2775, 2277-2770, 2277-2775, 2347-2611, |
| | 2349-2579, 2379-2677, 2419-2809, 2449-2719, |
| | 2482-2742, 2482-3056, 2500-2792, 2506-2799, |
| | 2557-2685, 2597-2867, 2605-2855, 2605-3177, |
| | 2633-2848, 2636-2895, 2663-2967, 2685-3013, |
| | 2697-2834, 2705-2974, 2767-3030, 2767-3043, |
| | 2771-3037, 2788-3016, 2871-3155, 2889-3172, |
| | 2900-3190, 2916-3126, 2916-3189, 2951-3190, |
| | 2957-3248, 2968-3206, 3025-3265, 3035-3285, |
| | 3051-3312, 3053-3370, 3118-3377, 3158-3398, |
| | 3218-3506, 3239-3484, 3271-3551, |
| | 3295-3530, 3303-3597, 3312-3528, 3312-3903, |
| | 3323-3599, 3330-3993, 3332-3998, 3333-3570, |
| | 3333-3571, 3333-3820, 3335-4006, 3356-4007, |
| | 3400-3633, 3400-3829, 3480-3743, 3480-4009, |
| | 3480-4017, 3582-3845, 3582-3976, 3582-4017, |
| | 3614-3840 |
| 43/2870383CB1/ | 1-1348, 1-1458, 446-766, 446-767, |
| 1570 | 508-1004, 1184-1570 |
| 44/1285088CB1/ | 1-284, 11-301, 24-284, 45-284, 244-1692, |
| 2642 | 303-686, 343-770, 430-647, 430-741, 430- |
| | 760, 430-780, 430-782, 430-783, 430-803, |
| | 430-881, 465-906, 707-1236, 730-1549, |
| | 791-1374, 869-1125, 1013-1282, 1043-1191, |
| | 1093-1649, 1181-1423, 1207-1367, 1231-1550, |
| | 1231-1576, 1373-1965, 1458-1877, 1568-1779, |
| | 1754-2426, 2014-2473, 2175-2466, 2237-2513, |
| | 2301-2582, 2330-2642 |
| 45/1532441CB1/ | 1-763, 80-320, 80-463, 100-148, 110-778, |
| 2618 | 146-463, 147-399, 256-324, 260-459, 464- |
| | 834, 470-1054, 475-1116, 495-953, 547-899, |
| | 824-1029, 884-1624, 970-1542, 1020-1659, |
| | 1057-1660, 1065-1753, 1066-1720, 1240-1911, |
| | 1325-1912, 1465-1896, 1509-1870, 1624-1911, |
| | 1716-2514, 1734-2027, 1957-2201, 2102-2618 |
| 46/3056408CB1/ | 1-510, 1-517, 1-613, 1-678, 11-363, |
| 6294 | 138-678, 179-519, 237-640, 397-1242, |
| | 499-1384, 687-1199, 736-1221, 839-1124, |
| | 881-1374, 893-1403, 977-1601, 1015-1403, |
| | 1037-1403, 1159-1416, 1162-1702, 1403-1885, |
| | 1520-2031, 1764-2041, 1817-2333, 1901-2512, |
| | 1926-2219, 2049-2235, 2103-2715, 2106-2553, |
| | 2201-2771, 2222-2794, 2302-3025, 2304-2777, |
| | 2305-2712, 2305-3027, 2380-3020, 2385-2771, |
| | 2436-3130, 2461-2773, 2542-2688, 2561-3141, |
| | 2575-3103, 2617-3347, 2666-3287, 2698-2845, |
| | 2793-3291, 2918-3462, 2940-3441, 3082-3317, |
| | 3226-3793, 3285-3475, 3334-3931, 3347-3517, |
| | 3414-3625, 3416-3639, 3416-3678, 3583-3832, |
| | 3584-4227, 3662-3912, 3669-4447, 3682-4208, |
| | 3685-3944, 3711-3981, 3724-4214, 3743-4334, |
| | 3778-4414, 3840-4559, 3884-4088, 3907-4502, |
| | 3914-4463, 4031-4559, 4036-4398, 4049-4748, |
| | 4049-4783, 4057-4570, 4082-4404, 4082-4511, |
| | 4086-4317, 4107-4369, 4148-4767, 4170-4706, |
| | 4188-4437, 4189-4493, 4214-4497, 4235-4595, |
| | 4249-4462, 4253-4499, 4263-4650, 4300-4562, |
| | 4309-4630, 4315-4673, 4372-4655, 4372-4776, |
| | 4387-4626, 4405-4631, 4438-4680, 4444-5010, |
| | 4475-5007, 4479-4715, 4512-5134, 4518-5119, |
| | 4518-5143, 4518-5144, 4518-5153, 4518-5174, |
| | 4521-4949, 4521-5101, 4521-5105, |
| | 4521-5125, 4521-5133, 4521-5143, 4521-5165, |
| | 4524-5163, 4531-5039, 4547-5025, 4548-4816, |
| | 4558-5123, 4559-4851, 4579-4850, 4585-5162, |
| | 4600-5110, 4635-4895, 4635-5077, 4681-4930, |
| | 4716-5324, 4736-5091, 4753-4962, 4753-5213, |
| | 4757-5329, 4763-5123, 4797-4920, 4797-5058, |
| | 4797-5155, 4797-5178, 4869-5318, 4883-5451, |
| | 4896-5432, 4904-5137, 4904-5529, 4958-5290, |
| | 4966-5410, 4967-5754, 4968-5231, 4973-5611, |
| | 4973-5638, 4978-5215, 4978-5475, 4998-5266, |
| | 5006-5230, 5006-5233, 5006-5289, 5006-5550, |
| | 5007-5305, 5029-5557, 5039-5302, 5040-5759, |
| | 5047-5297, 5054-5765, 5066-5312, 5073-5763, |
| | 5082-5763, 5095-5759, 5096-5759, 5103-5763, |
| | 5104-5386, 5108-5763, 5109-5751, 5111-5658, |
| | 5124-5763, 5125-5407, 5135-5763, 5136-5596, |
| | 5140-5606, 5150-5399, 5165-5445, 5165-5418, |
| | 5165-5419, 5165-5421, 5165-5424, 5165-5432, |
| | 5165-5434, 5165-5472, 5165-5487, 5165-5489, |
| | 5165-5498, 5168-5606, 5182-5595, 5192-5820, |
| | 5227-5498, 5229-5597, 5240-5708, 5272-5602, |
| | 5279-5597, 5280-5850, 5311-5763, 5327-5756, |
| | 5343-5977, 5373-5763, 5450-5602, 5453-5718, |
| | 5460-5722, 5460-5915, 5461-5736, 5464-5875, |
| | 5502-5967, 5510-5953, 5529-5983, 5531-5798, |
| | 5532-6067, 5539-5826, 5577-6038, 5598-6022, |
| | 5626-5864, |
| | 5626-6183, 5629-6244, 5635-5885, 5671-6068, |
| | 5689-5926, 5718-5747, 5721-6285, 5733-6294, |
| | 5741-5981, 5741-6294, 5755-5986, 5759-6294, |
| | 5767-6021, 5768-6285, 5769-6049, 5770-6294, |
| | 5776-6142, 5889-6149, 5961-6211, 6023-6261, |
| | 6066-6294 |

TABLE 5

| Polynucleotide | Incyte | Representative |
|---|---|---|
| SEQ ID NO: | Project ID: | Library |
| 24 | 4936875CB1 | SINTNOR01 |
| 25 | 264408CB1 | BRAINOT03 |
| 26 | 2181434CB1 | PENITUT01 |
| 27 | 1367252CB1 | TESTTUT02 |
| 28 | 5633694CB1 | NERDTDN03 |
| 29 | 7985981CB1 | UTRSTUC01 |
| 30 | 4706628CB1 | THYMNOT11 |
| 31 | 5790110CB1 | BRAYDIN03 |
| 32 | 2948827CB1 | LIVRFEE04 |
| 35 | 6113748CB1 | PANCTUT02 |
| 36 | 7474037CB1 | FIBPFEN06 |
| 37 | 2955646CB1 | KIDNFET01 |
| 38 | 1573006CB1 | BRAHTDR04 |
| 39 | 1336756CB1 | SPLNNOE01 |
| 40 | 71259816CB1 | SINTNOR01 |
| 41 | 3354130CB1 | PLACFER06 |
| 42 | 1797985CB1 | SINTNOT13 |
| 44 | 1285088CB1 | THYMNOE01 |
| 45 | 1532441CB1 | COLENOR03 |
| 46 | 3056408CB1 | TNFRDNV01 |

TABLE 6

| Library | Vector | Library Description |
|---|---|---|
| BRAHTDR04 | PCDNA2.1 | This random primed library was constructed using |
| | | RNA isolated archaecortex, anterior hippocampus |
| | | tissue removed from a 55-year-old Caucasian female |
| | | who died from cholangiocarcinoma. Pathology indicated |
| | | mild meningeal fibrosis predominately over the |
| | | convexities, scattered axonal spheroids in the white |
| | | matter of the cingulate cortex and the thalamus, and |
| | | a few scattered neurofibrillary tangles in the |
| | | entorhinal cortex and the periaqueductal gray region. |
| | | Pathology for the associated tumor tissue indicated |
| | | well-differentiated cholangiocarcinoma of the liver |
| | | with residual or relapsed tumor. Patient history |
| | | included cholangiocarcinoma, post-operative Budd- |
| | | Chiari syndrome, biliary ascites, hydrothorax, |
| | | dehydration, malnutrition, oliguria and acute renal |
| | | failure. Previous surgeries included cholecystectomy |
| | | and resection of 85% of the liver. |
| BRAINOT03 | PSPORT1 | Library was constructed using RNA isolated from brain |
| | | tissue removed from a 26-year-old Caucasian male during |
| | | cranioplasty and excision of a cerebral meningeal lesion. |
| | | Pathology for the associated tumor tissue indicated a |
| | | grade 4 oligoastrocytoma in the right fronto-parietal |
| | | part of the brain. |
| BRAYDIN03 | pINCY | This normalized library was constructed from 6.7 million |
| | | independent clones from a brain tissue library. Starting |
| | | RNA was made from RNA isolated from diseased hypothalamus |
| | | tissue removed from a 57-year-old Caucasian male who died |
| | | from a cerebrovascular accident. Patient history included |
| | | Huntington's disease and emphysema. The library was |
| | | normalized in 2 rounds using conditions adapted from |
| | | Soares et al., PNAS (1994) 91: 9228 and Bonaldo et al., |
| | | Genome Research (1996) 6: 791, except that a significantly |
| | | longer (48-hours/round) reannealing hybridization was used. |
| | | The library was linearized and recircularized to select |
| | | for insert containing clones. |
| COLENOR03 | PCDNA2.1 | Library was constructed using RNA isolated from colon |
| | | epithelium tissue removed from a 13-year-old Caucasian |
| | | female who died from a motor vehicle accident. |
| FIBPFEN06 | pINCY | The normalized prostate stromal fibroblast tissue libraries |
| | | were constructed from 1.56 million independent clones from a |
| | | prostate fibroblast library. Starting RNA was made from |
| | | fibroblasts of prostate stroma removed from a male fetus, |
| | | who died after 26 weeks' gestation. The libraries were |
| | | normalized in two rounds using conditions adapted from |
| | | Soares et al., PNAS (1994) 91: 9228 and Bonaldo et al., |
| | | Genome Research (1996) 6: 791, except that a significantly |
| | | longer (48-hours/round) reannealing hybridization was used. |
| | | The library was then linearized and recircularized to select |
| | | for insert containing clones as follows: plasmid DNA was |
| | | prepped from approximately 1 million clones from the |
| | | normalized prostate stromal fibroblast tissue libraries |
| | | following soft agar transformation. |
| KIDNFET01 | pINCY | Library was constructed using RNA isolated from kidney |
| | | tissue removed from a Caucasian female fetus, who died |
| | | at 17 weeks' gestation from anencephalus. |
| LIVRFEE04 | PCDNA2.1 | This 5′ biased random primed library was constructed using |
| | | RNA isolated from liver tissue removed from a Caucasian |
| | | male fetus who died from Patau's syndrome (trisomy 13) at |
| | | 20-weeks' gestation. Serology was negative. |
| NERDTDN03 | pINCY | This normalized dorsal root ganglion tissue library was |
| | | constructed from 1.05 million independent clones from a |
| | | dorsal root ganglion tissue library. Starting RNA was |
| | | made from dorsal root ganglion tissue removed from the |
| | | cervical spine of a 32-year-old Caucasian male who died |
| | | from acute pulmonary edema, acute bronchopneumonia, |
| | | bilateral pleural effusions, pericardial effusion, and |
| | | malignant lymphoma (natural killer cell type). The |
| | | patient presented with pyrexia of unknown origin, malaise, |
| | | fatigue, and gastrointestinal bleeding. Patient history |
| | | included probable cytomegalovirus infection, liver |
| | | congestion, and steatosis, splenomegaly, hemorrhagic |
| | | cystitis, thyroid hemorrhage, respiratory failure, |
| | | pneumonia of the left lung, natural killer cell lymphoma |
| | | of the pharynx, Bell's palsy, and tobacco and alcohol |
| | | abuse. Previous surgeries included colonoscopy, closed |
| | | colon biopsy, adenotonsillectomy, and nasopharyngeal |
| | | endoscopy and biopsy. Patient medications included |
| | | Diflucan (fluconazole), Deltasone (prednisone), |
| | | hydrocodone, Lortab, Alprazolam, |
| | | Reazodone, ProMace-Cytabom, Etoposide, Cisplatin, |
| | | Cytarabine, and dexamethasone. The patient received |
| | | radiation therapy and multiple blood transfusions. |
| | | The library was normalized in 2 rounds using conditions |
| | | adapted from Soares et al., PNAS (1994) 91: 9228-9232 |
| | | and Bonaldo et al.. Genome Research 6 (1996): 791, |
| | | except that a significantly longer (48 |
| | | hours/round) reannealing hybridization was used. |
| PANCTUT02 | pINCY | Library was constructed using RNA isolated from |
| | | pancreatic tumor tissue removed from a 45-year-old |
| | | Caucasian female during radical pancreaticoduodenectomy. |
| | | Pathology indicated a grade 4 anaplastic carcinoma. |
| | | Family history included benign hypertension, |
| | | hyperlipidemia and atherosclerotic coronary |
| | | artery disease. |
| PENTTUT01 | pINCY | Library was constructed using RNA isolated from tumor |
| | | tissue removed from the penis of a 64-year-old |
| | | Caucasian male during penile amputation. Pathology |
| | | indicated a fungating invasive grade 4 squamous cell |
| | | carcinoma involving the inner wall of the foreskin |
| | | and extending onto the glans penis. Patient history |
| | | included benign neoplasm of the large bowel, |
| | | atherosclerotic coronary artery disease, angina |
| | | pectoris, gout, and obesity. Family history included |
| | | malignant pharyngeal neoplasm, chronic lymphocytic |
| | | leukemia, and chronic liver disease. |
| PLACFER06 | pINCY | This random primed library was constructed using RNA |
| | | isolated from placental tissue removed from a Caucasian |
| | | fetus who died after 16 weeks' gestation from fetal |
| | | demise and hydrocephalus. Patient history included |
| | | umbilical cord wrapped around the head (3 times) and |
| | | the shoulders (1 time). Serology was positive for anti- |
| | | CMV. Family history included multiple pregnancies and |
| | | live births, and an abortion. |
| SINTNOR01 | PCDNA2.1 | This random primed library was constructed using RNA |
| | | isolated from small intestine tissue removed from a |
| | | 31-year-old Caucasian female during Roux-en-Y gastric |
| | | bypass. Patient history included clinical obesity. |
| SINTNOT13 | pINCY | Library was constructed using RNA isolated from ileum |
| | | tissue obtained from a 25-year-old Asian female during |
| | | a partial colectomy and temporary ileostomy. Pathology |
| | | indicated moderately active chronic ulcerative colitis, |
| | | involving colonic mucosa from the distal margin to the |
| | | ascending colon. Family history included hyperlipidemia, |
| | | depressive disorder, malignant cervical neoplasm, viral |
| | | hepatitis A, and depressive disorder. |
| SPLNNOE01 | PCDNA2.1 | This 5′ biased random primed library was constructed using |
| | | RNA isolated from the spleen tissue of a 2-year-old Hispanic |
| | | male, who died from cerebral anoxia. Past medical history and |
| | | serologies were negative. |
| TESTTUT02 | pINCY | Library was constructed using RNA isolated from testicular |
| | | tumor removed from a 31-year-old Caucasian male during |
| | | unilateral orchiectomy. Pathology indicated embryonal |
| | | carcinoma. |
| THYMNOE01 | PCDNA2.1 | This 5′ biased random primed library was constructed using |
| | | RNA isolated from thymus tissue removed from a 2-year-old |
| | | Caucasian female during a thymectomy and patch closure of |
| | | left atrioventricular fistula. Pathology indicated there |
| | | was no gross abnormality of the thymus. The patient presented |
| | | with congenital heart abnormalities. Patient history included |
| | | double inlet left ventricle and a rudimentary right ventricle, |
| | | pulmonary hypertension, cyanosis, subaortic stenosis, seizures, |
| | | and a fracture of the skull base. Patient medications included |
| | | Lasix and Captopril. Family history included reflux |
| | | neuropathy in the mother. |
| THYMNOT11 | pINCY | The library was constructed using RNA isolated from thymustissue |
| | | removed from a 2-year-old Caucasian female during a thymectomy |
| | | and patchclosure of left atrioventricular fistula. The patient |
| | | presented with congenitalheart abnormalities. Patient history |
| | | included double inlet left ventricle and arudimentary right |
| | | ventricle, pulmonary hypertension, cyanosis, subaortic stenosis, |
| | | seizures, and a fracture of the skull base. Family history |
| | | included refluxneuropathy. |
| TNFRDNV01 | PCR2-TOPOTA | Library was constructed using pooled cDNA from different donors. |
| | | cDNA was generated using mRNA isolated from pooled small |
| | | intestine tissue removed from a Caucasian male fetus (donor A) |
| | | who died at 23 weeks' gestation from premature birth; from lung |
| | | tissue removed from a Caucasian male fetus (donor B) who died |
| | | from fetal demise; from pleura tumor tissue removed from a |
| | | 55-year-old Caucasian female (donor C) during a complete |
| | | pneumonectomy; from frontal/parietal brain tumor tissue |
| | | removed from a 2-year-old Caucasian female (donor D) during |
| | | excision of cerebral meningeal lesion; from liver tumor tissue |
| | | removed from a 72-year-old Caucasian male (donor E) during |
| | | partial hepatectomy; from pooled fetal brain tissue removed |
| | | from a Caucasian male fetus (donor F) who was stillborn |
| | | with a hypoplastic left heart at 23 weeks' gestation and |
| | | from brain tissue removed from a Caucasian male fetus (donor G), |
| | | who died at 23 weeks' gestation from premature birth; from pooled |
| | | fetal kidney tissue removed from 59, 20-33-week-old male and |
| | | female fetuses who died from spontaneous abortion; from pooled |
| | | thymus tissue removed from |
| | | 9, 18-32-year-old male and female donors who died from sudden |
| | | death; and from pooled fetal liver tissue removed from 32, |
| | | 18-24-week-old male and female fetuses. For donor A, |
| | | serologies were negative. Family history included diabetes |
| | | in the mother. For donor B, Serologies were negative. For donor |
| | | C, pathology indicated grade 3 sarcoma most consistent with |
| | | leiomyosarcoma, uterine primary, forming a bosellated mass |
| | | replacing the right lower lobe and a portion of the middle lobe. |
| | | Multiple nodules comprising the tumor show near total necrosis. |
| | | Smooth muscle actin was positive. Estrogen receptor was negative |
| | | and progesterone receptor was positive. The patient presented |
| | | with shortness of breath. Patient history included peptic ulcer |
| | | disease, normal delivery, anemia, and tobacco abuse in remission. |
| | | Previous surgeries included total abdominal hysterectomy, |
| | | bilateral salpingo-oophorectomy, hemorrhoidectomy, endoscopic |
| | | excision of lung lesion, and appendectomy. Patient medications |
| | | included Megace, tamoxifen, and Pepcid. Family history included |
| | | multiple sclerosis in the mother; atherosclerotic coronary artery |
| | | disease and type II diabetes in the father; and breast cancer |
| | | in the grandparent(s) |
| | | For donor D, pathology indicated neuroectodermal tumor |
| | | with advanced ganglionic differentiation. The lesion |
| | | was only moderately cellular but was mitotically active |
| | | with a high MIB-1 labelling index. Neuronal differentiation |
| | | was widespread and advanced. Multinucleate and dysplastic- |
| | | appearing forms were readily seen. The glial element was |
| | | less prominent. The patient presented with motor |
| | | seizures. Family history included hypertension in |
| | | the grandparent(s). For donor E, pathology indicated |
| | | metastatic grade 2 (of 4) neuroendocrine carcinoma |
| | | forming a mass. The patient presented with metastatic |
| | | liver cancer. Patient history included benign hypertension, |
| | | type I diabetes, prostatic hyperplasia, prostate cancer, |
| | | alcohol abuse in remission, and tobacco abuse in remission. |
| | | Previous surgeries included destruction of a pancreatic |
| | | lesion, closed prostatic biopsy, transurethral prostatectomy, |
| | | removal of bilateral testes and total splenectomy. Patient |
| | | medications included Eulexin, Hytrin, Proscar, Ecotrin, and |
| | | insulin. Family history included atherosclerotic coronary |
| | | artery disease and acute myocardial infarction in the mother; |
| | | atherosclerotic coronary artery disease and type II diabetes |
| | | in the father. |
| | | For donor F and G, Serologies were negative for both donors |
| | | and family history for donor G included diabetes in the |
| | | mother. |
| UTRSTUC0l | PSPORT1 | This large size fractionated library was constructed using |
| | | pooled cDNA from two donors. cDNA was generated using |
| | | mRNA isolated from uterus tumor tissue removed from a |
| | | 37-year-old Black female (donor A) during myomectomy, |
| | | dilation and curettage, right fimbrial region biopsy, and |
| | | incidental appendectomy; and from endometrial tumor tissue |
| | | removed from a 49-year-old Caucasian female (donor B) during |
| | | vaginal hysterectomy and bilateral salpingo-oophorectomy. |
| | | For donor A, pathology indicated multiple uterine leiomyomata. |
| | | A fimbrial cyst was identified. The endometrium was in |
| | | secretory phase with hormonal effect. The patient presented |
| | | with deficiency anemia, an umbilical hernia, and |
| | | premenopausal menorrhagia. Patient history included |
| | | premenopausal menorrhagia and sarcoidosis of the lung. |
| | | Previous surgeries included hysteroscopy, dilation and |
| | | curettage, and endoscopic lung biopsy. Patient medications |
| | | included Chromagen and Claritin. For donor B, pathology |
| | | indicated grade 3 adenosquamous carcinoma forming a mass |
| | | within the |
| | | uterine fundus and involving the anterior uterine wall, |
| | | as well as focally involving an adjacent endometrial polyp. |
| | | The tumor invaded to a maximum depth of 7mm (uninvolved |
| | | wall thickness, 2.2cm). The adjacent endometrium was inactive. |
| | | Paraffin section immunostains for estrogen receptors and |
| | | progesterone receptors were positive. Patient history included |
| | | malignant breast neoplasm. Previous surgeries included |
| | | unilateral extended simple mastectomy and bilateral tubal |
| | | destruction. Patient medications included Megase and CAF |
| | | (Cyclophosphamide, Adriamycin, Fluoroacil). |

TABLE 7

| Program | Description | Reference | Parameter Threshold |
|---|---|---|---|
| ABI FACTURA | A program that removes | Applied Biosystems, | |
| | vector sequences and masks | Foster City, CA. | |
| | ambiguous bases in nucleic | | |
| | acid sequences. | | |
| ABI/PARACEL FDF | A Fast Data Finder useful | Applied Biosystems, | Mismatch <50% |
| | in comparing and annotating | Foster City, CA; Paracel | |
| | amino acid or nucleic | Inc., Pasadena, CA. | |
| | acid sequences. | | |
| ABI AutoAssembler | A program that assembles | Applied Biosystems, | |
| | nucleic acid sequences. | Foster City, CA. | |
| BLAST | A Basic Local Alignment | Altschul, S. F. et al. | ESTs: Probability value = |
| | Search Tool useful in | (1990) J. Mol. Biol. 215: | 1.0E−8 |
| | sequence similarity search | 403-410; Altschul, S. F. | or less Full Length sequences: |
| | for amino acid and nucleic | et al. (1997) Nucleic | Probability value = 1.0E−10 |
| | acid sequences. BLAST | Acids Res. 25: 3389-3402. | or less |
| | includes five functions: | | |
| | blastp, blastn, blastx, | | |
| | tblastn, and tblastx. | | |
| FASTA | A Pearson and Lipman | Pearson, W. R. and D. J. | ESTs: fasta E value = 1.06E−6 |
| | algorithm that searches for | Lipman (1988) Proc. | Assembled ESTs: fasta Identity= 95% |
| | similarity between a query | Natl. Acad Sci. USA 85: | or greater and Match length = 200 |
| | sequence and a group of | 2444-2448; Pearson, | bases or greater; fastx E value = |
| | sequences of the same type. | W. R. (1990) Methods | 1.0E−8 or less Full Length |
| | FASTA comprises as least | Enzymol. 183: 63-98; and | sequences: fastx score = |
| | five functions: fasta, tfasta, | Smith, T. F. and M. S. | 100 or greater |
| | fastx, tfastx, and ssearch. | Waterman (1981) Adv. | |
| | | Appl. Math. 2: 482-489. | |
| BLIMPS | A BLocks IMProved Searcher | Henikoff, S. and J. G. | Probability value = 1.0E−3 or |
| | that matches a sequence | Henikoff (1991) Nucleic | less |
| | against those in BLOCKS, | Acids Res. 19: 6565-6572; | |
| | PRINTS, DOMO, PRODOM, and | Henikoff, J. G. and | |
| | PFAM databases to search | S. Henikoff (1996) Methods | |
| | for gene families, sequence | Enzymol. 266: 88- | |
| | homology, and structural | 105; and Attwood, T. K. et | |
| | fingerprint regions. | al. (1997) J. Chem. | |
| | | Inf. Comput. Sci. 37: | |
| | | 417-424. | |
| HMMER | An algorithm for searching | Krogh, A. et al. (1994) J. | PFAM, INCY, SMART, or |
| | a query sequence against | Mol. Biol. 235: 1501- | TIGRFAM hits: Probability |
| | hidden Markov model (HMM)- | 1531; Sonnhammer, E. L. L. et | value = 1.0E−3 |
| | based databases of protein | al. (1988) Nucleic Acids | or less Signal peptide |
| | family consensus sequences, | Res. 26: 320-322; Durbin, R. et | hits: Score = 0 or |
| | such as PFAM, INCY, SMART, | al. (1998) Our World View, | greater |
| | and TIGRFAM. | in a Nutshell, Cambridge Univ. | |
| | | Press, pp. 1-350. | |
| ProfileScan | An algorithm that searches | Gribskov, M. et al. (1988) | Normalized quality scores > |
| | for structural and sequence | CABIOS 4: 61-66; Gribskov, | GCG-specified “HIGH” |
| | motifs in protein sequences | M. et al. (1989) Methods | value for that particular |
| | that match sequence patterns | Enzymol. 183: 146-159; | Prosite motif. Generally, |
| | defined in Prosite. | Bairoch, A. et al. (1997) | score = 1.4-2.1. |
| | | Nucleic Acids Res. | |
| | | 25: 217-221. | |
| Phred | A base-calling algorithm that | Ewing, B. et al. (1998) | |
| | examines automated sequencer | Genome Res. 8: 175-185; | |
| | traces with high sensitivity | Ewing, B. and P. Green | |
| | and probability. | (1998) Genome | |
| | | Res. 8: 186-194. | |
| Phrap | A Phils Revised Assembly Program | Smith, T. F. and M. S. | Score = 120 or greater; |
| | including SWAT and CrossMatch, | Waterman (1981) Adv. | Match length = 56 or |
| | programs based on efficient | Appl. Math. 2: 482-489; | greater |
| | implementation of the Smith- | Smith, T. F. and M. S. | |
| | Waterman algorithm, useful in | Waterman (1981) J. Mol. | |
| | searching sequence homology and | Biol. 147: 195-197; | |
| | assembling DNA sequences. | and Green, P., University | |
| | | of Washington, | |
| | | Seattle, WA. | |
| Consed | A graphical tool for viewing | Gordon, D. et al. (1998) | |
| | and editing Phrap | Genome Res. 8: 195-202. | |
| | assemblies. | | |
| SPScan | A weight matrix analysis | Nielson, H. et al. (1997) | Score = 3.5 or greater |
| | program that scans protein | Protein Engineering | |
| | sequences for the presence | 10: 1-6; Claverie, J. M. | |
| | of secretory signal peptides. | and S. Audic (1997) | |
| | | CABIOS 12: 431-439. | |
| TMAP | A program that uses weight | Persson, B. and P. Argos | |
| | matrices to delineate | (1994) J. Mol. Biol. | |
| | transmembrane segments | 237: 182-192; Persson, | |
| | on protein sequences and | B. and P. Argos (1996) | |
| | determine orientation. | Protein Sci. 5: 363-371. | |
| TMHMMER | A program that uses a | Sonnhammer, E. L. et al. | |
| | hidden Markov model (HMM) | (1998) Proc. Sixth Intl. | |
| | to delineate transmembrane | Conf. on Intelligent | |
| | segments on protein | Systems for Mol. Biol., | |
| | sequences and determine | Glasgow et al., eds., | |
| | orientation. | The Am. Assoc. for | |
| | | Artificial Intelligence | |
| | | Press, Menlo Park, CA, | |
| | | pp. 175-182. | |
| Motifs | A program that searches | Bairoch, A. et al. (1997) | |
| | amino acid sequences for | Nucleic Acids Res. 25: | |
| | patterns that matched | 217-221; Wisconsin Package | |
| | those defined in Prosite. | Program Manual, | |
| | | version 9, page M51-59, | |
| | | Genetics Computer | |
| | | Group, Madison, WI. | |

What is claimed is:

1. An isolated polypeptide selected from the group consisting of:
a) a polypeptide comprising an amino acid sequence selected from the group consisting of SEQ ID NO:1-23,
b) a polypeptide comprising a naturally occurring amino acid sequence at least 90% identical to an amino acid sequence selected from the group consisting of SEQ ID NO:1-12, SEQ ID NO:14-18, SEQ ID NO:20 and SEQ ID NO:23,
c) a polypeptide comprising a naturally occurring amino acid sequence at least 99% identical to the amino acid sequence of SEQ ID NO:21,
d) a polypeptide comprising a naturally occurring amino acid sequence at least 98% identical to the amino acid sequence of SEQ ID NO:22,
e) a biologically active fragment of a polypeptide having an amino acid sequence selected from the group consisting of SEQ ID NO:1-23, and
f) an immunogenic fragment of a polypeptide having an amino acid sequence selected from the group consisting of SEQ ID NO:1-23.

2. An isolated polypeptide of claim 1 comprising an amino acid sequence selected from the group consisting of SEQ ID NO:1-23.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:24-46.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-23.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:24-46,
b) a polynucleotide comprising a naturally occurring polynucleotide sequence at least 90% identical to a polynucleotide sequence selected from the group consisting of SEQ ID NO:24-35, SEQ ID NO:37-41 and SEQ ID NO:43-46,
c) a polynucleotide comprising a naturally occurring polynucleotide sequence at least 96% identical to the polynucleotide sequence of SEQ ID NO:42,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. An isolated polynucleotide comprising at least 60 contiguous nucleotides of a polynucleotide of claim 12.

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. A method of claim 14, wherein the probe comprises at least 60 contiguous nucleotides.

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-23.

19. A method for treating a disease or condition associated with decreased expression of functional NAAP, comprising administering to a patient in need of such treatment the composition of claim 17.

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. A composition comprising an agonist compound identified by a method of claim 20 and a pharmaceutically acceptable excipient.

22. A method for treating a disease or condition associated with decreased expression of functional NAAP, comprising administering to a patient in need of such treatment a composition of claim 21.

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. A composition comprising an antagonist compound identified by a method of claim 23 and a pharmaceutically acceptable excipient.

25. A method for treating a disease or condition associated with overexpression of functional NAAP, comprising administering to a patient in need of such treatment a composition of claim 24.

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30. A diagnostic test for a condition or disease associated with the expression of NAAP in a biological sample, the method comprising:
a) combining the biological sample with an antibody of claim 11, under conditions suitable for the antibody to bind the polypeptide and form an antibody:polypeptide complex, and
b) detecting the complex, wherein the presence of the complex correlates with the presence of the polypeptide in the biological sample.

31. The antibody of claim 11, wherein the antibody is:
a) a chimeric antibody,
b) a single chain antibody,
c) a Fab fragment,
d) a F(ab′)₂ fragment, or
e) a humanized antibody.

32. A composition comprising an antibody of claim 11 and an acceptable excipient.

33. A method of diagnosing a condition or disease associated with the expression of NAAP in a subject, comprising administering to said subject an effective amount of the composition of claim 32.

34. A composition of claim 32, wherein the antibody is labeled.

35. A method of diagnosing a condition or disease associated with the expression of NAAP in a subject, comprising administering to said subject an effective amount of the composition of claim 34.

36. A method of preparing a polyclonal antibody with the specificity of the antibody of claim 11, the method comprising:
a) immunizing an animal with a polypeptide consisting of an amino acid sequence selected from the group consisting of SEQ ID NO:1-23, or an immunogenic fragment thereof, under conditions to elicit an antibody response,
b) isolating antibodies from said animal, and
c) screening the isolated antibodies with the polypeptide, thereby identifying a polyclonal antibody which specifically binds to a polypeptide comprising an amino acid sequence selected from the group consisting of SEQ ID NO:1-23.

37. A polyclonal antibody produced by a method of claim 36.

38. A composition comprising the polyclonal antibody of claim 37 and a suitable carrier.

39. A method of making a monoclonal antibody with the specificity of the antibody of claim 11, the method comprising:
a) immunizing an animal with a polypeptide consisting of an amino acid sequence selected from the group consisting of SEQ ID NO:1-23, or an immunogenic fragment thereof, under conditions to elicit an antibody response,
b) isolating antibody producing cells from the animal,
c) fusing the antibody producing cells with immortalized cells to form monoclonal antibody-producing hybridoma cells,
d) culturing the hybridoma cells, and
e) isolating from the culture monoclonal antibody which specifically binds to a polypeptide comprising an amino acid sequence selected from the group consisting of SEQ ID NO:1-23.

40. A monoclonal antibody produced by a method of claim 39.

41. A composition comprising the monoclonal antibody of claim 40 and a suitable carrier.

42. The antibody of claim 11, wherein the antibody is produced by screening a Fab expression library.

43. The antibody of claim 11, wherein the antibody is produced by screening a recombinant immunoglobulin library.

44. A method of detecting a polypeptide comprising an amino acid sequence selected from the group consisting of SEQ ID NO:1-23 in a sample, the method comprising:
a) incubating the antibody of claim 11 with a sample under conditions to allow specific binding of the antibody and the polypeptide, and
b) detecting specific binding, wherein specific binding indicates the presence of a polypeptide comprising an amino acid sequence selected from the group consisting of SEQ ID NO:1-23 in the sample.

45. A method of purifying a polypeptide comprising an amino acid sequence selected from the group consisting of SEQ ID NO:1-23 from a sample, the method comprising:
a) incubating the antibody of claim 11 with a sample under conditions to allow specific binding of the antibody and the polypeptide, and
b) separating the antibody from the sample and obtaining the purified polypeptide comprising an amino acid sequence selected from the group consisting of SEQ ID NO:1-23.

46. A microarray wherein at least one element of the microarray is a polynucleotide of claim 13.

47. A method of generating an expression profile of a sample which contains polynucleotides, the method comprising:
a) labeling the polynucleotides of the sample,
b) contacting the elements of the microarray of claim 46 with the labeled polynucleotides of the sample under conditions suitable for the formation of a hybridization complex, and
c) quantifying the expression of the polynucleotides in the sample.

48. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, and wherein said target polynucleotide is a polynucleotide of claim 12.

49. An array of claim 48, wherein said first oligonucleotide or polynucleotide sequence is completely complementary to at least 30 contiguous nucleotides of said target polynucleotide.

50. An array of claim 48, wherein said first oligonucleotide or polynucleotide sequence is completely complementary to at least 60 contiguous nucleotides of said target polynucleotide.

51. An array of claim 48, wherein said first oligonucleotide or polynucleotide sequence is completely complementary to said target polynucleotide.

52. An array of claim 48, which is a microarray.

53. An array of claim 48, further comprising said target polynucleotide hybridized to a nucleotide molecule comprising said first oligonucleotide or polynucleotide sequence.

54. An array of claim 48, wherein a linker joins at least one of said nucleotide molecules to said solid substrate.

55. An array of claim 48, wherein each distinct physical location on the substrate contains multiple nucleotide molecules, and the multiple nucleotide molecules at any single distinct physical location have the same sequence, and each distinct physical location on the substrate contains nucleotide molecules having a sequence which differs from the sequence of nucleotide molecules at another distinct physical location on the substrate.

56. A polypeptide of claim 1, comprising the amino acid sequence of SEQ ID NO:1.

57. A polypeptide of claim 1, comprising the amino acid sequence of SEQ 1D NO:2.

58. A polypeptide of claim 1, comprising the amino acid sequence of SEQ ID NO:3.

59. A polypeptide of claim 1, comprising the amino acid sequence of SEQ ID NO:4.

60. A polypeptide of claim 1, comprising the amino acid sequence of SEQ ID NO:5.

61. A polypeptide of claim 1, comprising the amino acid sequence of SEQ. ID NO:6.

62. A polypeptide of claim 1, comprising the amino acid sequence of SEQ ID NO:7.

63. A polypeptide of claim 1, comprising the amino acid sequence of SEQ ID NO:8.

64. A polypeptide of claim 1, comprising the amino acid sequence of SEQ ID NO:9.

65. A polypeptide of claim 1, comprising the amino acid sequence of SEQ I) NO:10.

66. A polypeptide of claim 1, comprising the amino acid sequence of SEQ I) NO:11.

67. A polypeptide of claim 1, comprising the amino acid sequence of SEQ ID NO:12.

68. A polypeptide of claim 1, comprising the amino acid sequence of SEQ ID NO:13.

69. A polypeptide of claim 1, comprising the amino acid sequence of SEQ ID NO:14.

70. A polypeptide of claim 1, comprising the amino acid sequence of SEQ ID NO:15.

71. A polypeptide of claim 1, comprising the amino acid sequence of SEQ ID NO:16.

72. A polypeptide of claim 1, comprising the amino acid sequence of SEQ ID NO:17.

73. A polypeptide of claim 1, comprising the amino acid sequence of SEQ ID NO:18.

74. A polypeptide of claim 1, comprising the amino acid sequence of SEQ ID NO:19.

75. A polypeptide of claim 1, comprising the amino acid sequence of SEQ ID NO:20.

76. A polypeptide of claim 1, comprising the amino acid sequence of SEQ D) NO:21.

77. A polypeptide of claim 1, comprising the amino acid sequence of SEQ ID NO:22.

78. A polypeptide of claim 1, comprising the amino acid sequence of SEQ ID NO:23.

79. A polynucleotide of claim 12, comprising the polynucleotide sequence of SEQ ID NO:24.

80. A polynucleotide of claim 12, comprising the polynucleotide sequence of SEQ ID NO:25.

81. A polynucleotide of claim 12, comprising the polynucleotide sequence of SEQ ID NO:26.

82. A polynucleotide of claim 12, comprising the polynucleotide sequence of SEQ ID NO:27.

83. A polynucleotide of claim 12, comprising the polynucleotide sequence of SEQ ID NO:28.

84. A polynucleotide of claim 12, comprising the polynucleotide sequence of SEQ ID NO:29.

85. A polynucleotide of claim 12, comprising the polynucleotide sequence of SEQ ID NO:30.

86. A polynucleotide of claim 12, comprising the polynucleotide sequence of SEQ ID NO:31.

87. A polynucleotide of claim 12, comprising the polynucleotide sequence of SEQ ID NO:32.

88. A polynucleotide of claim 12, comprising the polynucleotide sequence of SEQ ID NO:33.

89. A polynucleotide of claim 12, comprising the polynucleotide sequence of SEQ ID NO:34.

90. A polynucleotide of claim 12, comprising the polynucleotide sequence of SEQ ID NO:35.

91. A polynucleotide of claim 12, comprising the polynucleotide sequence of SEQ ID NO:36.

92. A polynucleotide of claim 12, comprising the polynucleotide sequence of SEQ ID NO:37.

93. A polynucleotide of claim 12, comprising the polynucleotide sequence of SEQ ID NO:38.

94. A polynucleotide of claim 12, comprising the polynucleotide sequence of SEQ ID NO:39.

95. A polynucleotide of claim 12, comprising the polynucleotide sequence of SEQ ID NO:40.

96. A polynucleotide of claim 12, comprising the polynucleotide sequence of SEQ ID NO:41.

97. A polynucleotide of claim 12, comprising the polynucleotide sequence of SEQ ID NO:42.

98. A polynucleotide of claim 12, comprising the polynucleotide sequence of SEQ ID NO:43.

99. A polynucleotide of claim 12, comprising the polynucleotide sequence of SEQ ID NO:44.

100. A polynucleotide of claim 12, comprising the polynucleotide sequence of SEQ ID NO:45.

101. A polynucleotide of claim 12, comprising the polynucleotide sequence of SEQ ID NO:46.
